(19) United States
(12) Patent Application Publication
(10) Pub. No.: US 2026/0346238 A1
(43) Pub. Date: Nov. 5, 2026

(54) CATALYST FOR EPOXIDATION OF PROPYLENE

(71) Applicant: BASF SE, Ludwigshafen am Rheim (DE)

(72) Inventors: Trees Maria DE BAERDEMAEKER, Ludwigshafen am Rhein (DE); Andrei-Nicolae PARVULESCU, Ludwigshafen am Rhein (DE); Milan KOSTUR, Ludwigshafen am Rhein (DE); Joaquim Henrique TELES, Ludwigshafen am Rhein (DE); Jaroslaw Michael MORMUL, Ludwigshafen am Rhein (DE); Joris WIEME, Antwerpen (BE); Nikolaus POTH, Antwerpen (BE); Gerrit WATERS, Ludwigshafen am Rhein (DE)

(21) Appl. No.: 19/487,665

(22) PCT Filed: Feb. 5, 2024
(86) PCT No.: PCT/EP2024/052769

§ 371 Date: Nov. 25, 2025

(30) Foreign Application Priority Data
May 31, 2023 (EP) 23176534.8

(51) Int. Cl. B01J 29/89; B01J 35/32; B01J 35/37; B01J 35/61; B01J 35/63; C07D 301/12

(52) U.S. Cl. CPC B01J 29/89; B01J 35/32; C07D 301/12; B01J 35/37; B01J 35/615; B01J 35/635; B01J 35/638; B01J 2229/42; B01J 2235/05

(57) ABSTRACT

The present invention relates to a catalyst molding comprising a zeolitic material having framework type MFI, wherein the catalyst molding has a specific average crush strength, and wherein the catalyst molding has a specific aspect ratio. Further, the present invention relates to a reactor comprising a plurality of said catalyst moldings. Yet further, the present invention relates to a process for preparing said catalyst molding, and a catalyst molding obtained or obtainable by said process. Yet further, the present invention relates to a process for the activation of hydrogen peroxide, to a use of said catalyst molding, as a catalyst and/or catalyst component, or of said reactor, in a reaction involving one or more of C—O bond formation, C—C bond formation and C—C bond conversion. Yet further, the present invention relates to a process for preparing an olefin oxide.

### TECHNICAL FIELD

The present invention relates to a catalyst molding comprising a zeolitic material having framework type MFI, wherein the catalyst molding has a specific aspect ratio. Furthermore, the present invention relates to a method for preparing a catalyst molding. Yet further, the present invention relates to a process for the activation of hydrogen peroxide, and use of the inventive catalyst molding in a reaction involving one or more of C—O bond formation, C—C bond formation and C—C bond conversion.

### INTRODUCTION

Titanium containing zeolitic materials of structure type MFI are known to be efficient catalysts including, for example, epoxidation reactions. In such industrial-scale processes, typically carried out in continuous mode, these zeolitic materials are usually employed in the form of moldings which, in addition to the catalytically active zeolitic material, comprise a suitable binder.

US 2015/0118149 A1 and CN 115974094 A relate to a titanium silicalite molecular sieves and their synthesis. CN 115920958 A relates to a modification method of a titanium-silicon molecular sieve and application thereof, wherein the titanium-silicon molecular sieve is treated with a treatment liquid, the treatment liquid being a mixture of cyclic imine, quaternary ammonium salt, organic amine salt and water, the titanium-silicon molecular sieve can be TS-1. WO 2015/029055 A1 relates to the field of forming or shaping of titanium silicalite (TS-1) catalysts. It is disclosed therein that a TS-1 material can be shaped by means of an operation such as extrusion. WO 2020/074586 A1 relates to a molding comprising a zeolitic material having framework type MFI, and discloses a process for preparing propylene oxide in the presence of a catalyst comprising said molding. R. Wang et al. give an overview on hollow MFI-type zeolites in their review on “Fundamental Understanding and Catalytic Applications of Hollow MFI-type Zeolites” in Catalysis Today 2022.

WO 2015/059171 A1 discloses a molding for a hydrophobic zeolitic material and process for its production.

There however remains a need for the provision of improved catalyst moldings for the activation of hydrogen peroxide, in particular with regard to their catalytic efficiency in epoxidation reactions, and more generally in oxidation reactions based on the conversion of hydrogen peroxide. In particular, there remains a need for an improved process for the activation of hydrogen peroxide where such improved catalyst moldings may be provided.

### DETAILED DESCRIPTION

Thus, it was an object of the present invention to provide a novel catalyst molding comprising a zeolitic material having framework type MFI, wherein the catalyst molding has a specific aspect ratio. It was a further object to provide a novel catalyst molding, wherein said molding has advantageous characteristics, in particular an improved hydrogen peroxide activation ability, more particularly an improved propylene oxide selectivity when used as a catalyst or catalyst component, in particular in the epoxidation reaction of propylene to propylene oxide. It was a further object of the present invention to provide a process for the preparation of such a catalyst molding, in particular to provide a process resulting in a catalyst molding having advantageous properties, preferably when used as a catalyst or catalyst component, specifically in an oxidation or epoxidation reaction. It was a further object of the present invention to provide an improved process for the epoxidation of propylene with hydrogen peroxide as oxidizing agent, allowing for a very high propylene selectivity.

Surprisingly, it was found that such a catalyst molding exhibiting said advantageous characteristics can be provided if a given zeolitic material having framework type MFI is subjected to a specific shaping process, resulting in a catalyst molding particularly having a specific aspect ratio and a specific crush strength. In particular, it has surprisingly been found that a catalyst molding can be provided which shows, if used as a catalyst in an epoxidation reaction of propylene to propylene oxide, significantly increased propylene oxide selectivity and yield, and further exhibits excellent life time properties.

Therefore, the present invention relates to a catalyst molding comprising a zeolitic material having framework type MFI, wherein the catalyst molding has an average crush strength in the range of from 3 to 30 N, wherein the crush strength is preferably determined according to Reference Example 1.7,
wherein the catalyst molding has an aspect ratio D1:D2,
wherein D1 stands for the largest distance separating a pair of parallel planes P1 and P2 when the catalyst molding is located in-between said parallel planes without transgressing them, and the catalyst molding is in contact with each of the respective planes in at least one point, and wherein D2 stands for the shortest distance separating a pair of parallel planes P3 and P4 when the catalyst molding is located in-between said parallel planes without transgressing them, and the catalyst molding is in contact with each of the respective planes in at least one point, wherein the aspect ratio D1:D2 is equal to or greater than 1:1, wherein the aspect ratio is preferably determined according to Reference Example 1.12.

It is preferred that the aspect ratio D1:D2 is in the range of from 1.4:1 to 6.1:1, more preferably in the range of from 1.45:1 to 5.6:1, more preferably in the range of from 1.5:1 to 5.1:1, more preferably in the range of from 1.55:1 to 4.6:1, more preferably in the range of from 1.6:1 to 4.1:1, more preferably in the range of from 1.65:1 to 3.6:1, more preferably in the range of from 1.7:1 to 3.2:1, more preferably in the range of from 1.75:1 to 2.7:1, more preferably in the range of from 1.8:1 to 2.2:1, wherein the aspect ratio is preferably determined according to Reference Example 1.12.

It is preferred that D1 is in the range of from 0.1 to 10 mm, more preferably in the range of from 1.2 to 7.0 mm, more preferably in the range of from 2.2 to 5.0 mm, more preferably in the range of from 3.0 to 4.0 mm, more preferably in the range of from 3.4 to 3.8 mm.

It is preferred that D2 is in the range of from 0.05 to 5 mm, more preferably in the range of from 0.6 to 3.5 mm, more preferably in the range of from 1.1 to 2.5 mm, more preferably in the range of from 1.5 to 2.0 mm, more preferably in the range of from 1.7 to 1.9 mm.

It is preferred that the catalyst molding has a cross-sectional profile, wherein the cross-sectional profile is circular, hexagonal, rectangular, quadratic, triangular, oval, a star-shaped polygon, or cloverleaf-shaped, more preferably circular, hexagonal, rectangular, quadratic, triangular, oval, a star-shaped polygon having 3, 4, 5, 6, 7, or 8 tips, a trilobe or a quadrilobe, more preferably circular, hexagonal, rectangular, quadratic, triangular, oval, a star-shaped polygon having 3 or 4 tips, a trilobe or a quadrilobe. It is particularly preferred that the cross-sectional profile is a star-shaped polygon having 3 tips, wherein the tips are rounded and wherein the corners are rounded.

It is preferred that P3 and P1 form an angle in the range of from 60° to 90°, preferably in the range of from 70° to 90°, more preferably in the range of from 80° to 90°, more preferably in the range of from 85° to 90°, more preferably in the range of from 89° to 90°.

If P3 and P1 form an angle in the range of from 60° to 90°, then P3 and P2 also form an angle in the range of from 60° to 90° and, consequently, also P4 and P1 form an angle in the range of from 60° to 90°, and also P4 and P2 form an angle in the range of from 60° to 90°.

It is preferred that the catalyst molding has the shape of an extrudate, said shape having a length in the conceived or actual direction of extrusion, and said shape having a fixed cross-sectional profile perpendicular to said conceived or actual direction of extrusion.

It is particularly preferred that the catalyst molding having the shape of an extrudate has a circular cross-sectional profile with a diameter D, the diameter D corresponding to D2.

It is preferred that the catalyst molding has a total pore volume in the range of from 0.50 to 1.2 ml/g, more preferably in the range of from 0.60 to 1.2 ml/g, more preferably in the range of from 0.65 to 1.1 ml/g, more preferably in the range of from 0.70 to 1.0 ml/g.

It is preferred that the catalyst molding exhibits an average crush strength in the range of from 3 to 30 N, more preferably in the range of from 4 to 25 N, more preferably in the range of from 5 to 20 N, more preferably in the range of from 5 to 15 N, wherein the average crush strength is preferably determined according to Reference Example 1.7.

It is preferred that the catalyst molding further comprises one or more oxidic binders, wherein the one or more oxidic binders are more preferably selected from the group consisting of inorganic binders, wherein the one or more binders more preferably comprise one or more sources of a metal oxide and/or of a metalloid oxide, more preferably one or more sources of a metal oxide and/or of a metalloid oxide selected from the group consisting of silica, alumina, titania, zirconia, lanthana, magnesia, and mixtures and/or mixed oxides of two or more thereof, more preferably from the group consisting of silica, alumina, titania, zirconia, magnesia, silica-alumina mixed oxides, silica-titania mixed oxides, silica-zirconia mixed oxides, silica-lanthana mixed oxides, silica-zirconia-lanthana mixed oxides, alumina-titania mixed oxides, alumina-zirconia mixed oxides, alumina-lanthana mixed oxides, alumina-zirconia-lanthana mixed oxides, titania-zirconia mixed oxides, and mixtures and/or mixed oxides of two or more thereof, more preferably from the group consisting of silica, alumina, silica-alumina mixed oxides, and mixtures of two or more thereof, wherein more preferably the one or more oxidic binders comprise one or more sources of silica, wherein more preferably the one or more binders consist of one or more sources of silica.

In the case where the catalyst molding further comprises one or more oxidic binders, it is preferred that the catalyst molding comprises the one or more oxidic binders, calculated as the oxide, in an amount in the range of from 5 to 40 weight-%, more preferably of from 10 to 30 weight-%, more preferably of from 15 to 25 weight-%, more preferably of from 16 to 20 weight-%, based on the weight of the catalyst molding.

Further in the case where the catalyst molding further comprises one or more oxidic binders, it is preferred that from 95 to 100 weight-%, more preferably from 98 to 100 weight-%, more preferably from 99 to 100 weight-%, more preferably from 99.5 to 100 weight-%, more preferably from 99.9 to 100 weight-% of the catalyst molding consist of the zeolitic material and the one or more oxidic binders.

It is preferred that the catalyst molding exhibits a water adsorption in the range of from 1.0 to 15.0 weight-%, more preferably in the range of from 1.25 to 10.0 weight-%, more preferably in the range of from 1.5 to 8.0 weight-%, more preferably in the range of from 2.5 to 7.0 weight-%, more preferably in the range of from 3.5 to 6.5 weight-%, more preferably in the range of from 4.0 to 6.2 weight-%, more preferably in the range of from 4.3 to 6.0 weight-%, more preferably in the range of from 4.5 to 5.8 weight-%, wherein the water adsorption is preferably determined according to Reference Example 1.1. Alternatively, it is particularly preferred that the catalyst molding exhibits a water adsorption in the range of from 3 to 6.5 wt.-%, preferably of from 3.5 to 6 wt.-%, more preferably of from 4.4 to 5.5 wt.-%.

It is preferred that the catalyst molding has a Ti content in the range of from 0.4 to 1.85 weight-%, more preferably in the range of from 0.5 to 1.7 weight-%, more preferably in the range of from 0.6 to 1.5 weight-%, more preferably in the range of from 0.7 to 1.3 weight-%, more preferably in the range of from 0.8 to 1.2 weight-%, calculated as elemental Ti and based on the sum of the weights of the zeolitic material and optionally the one or more oxidic binders.

It is preferred that the catalyst molding has a Si content in the range of from 36 to 48 weight-%, more preferably in the range of from 38 to 46 weight-%, more preferably in the range of from 39 to 45 weight-%, calculated as elemental Si and based on the sum of the weights of the zeolitic material and optionally the one or more oxidic binders.

It is preferred that the catalyst molding has a crystallinity in the range of from 40 to 90 weight.-%, more preferably in the range of from 45 to 100 weight-%, more preferably in the range of from 50 to 90 weight-%, more preferably in the range of from 50 to 80 weight-%, wherein the crystallinity is preferably determined according to Reference Example 1.3.

It is preferred that the catalyst molding displays a water adsorption (W), preferably determined according to Reference Example 1.1, a concentration (C) of bridging μ² η²-peroxo species per Ti in the H₂O₂-activated catalyst molding, as determined by quantitative 170 NMR spectroscopy, preferably determined according to Reference Example 1.2, and an activation factor (A) according to formula I, wherein the activation factor is in the range of from 10 to 75 mmol/mol;

wherein in accordance with formula I, the activation factor is the multiplication product of the water adsorption and the concentration of bridging μ² η²-peroxo species per Ti in the H₂O₂-activated catalyst molding:

A=W×C (I).

In the case where the catalyst molding displays a water adsorption (W), a concentration (C) of bridging μ² η²-peroxo species per Ti in the H₂O₂-activated catalyst molding, and an activation factor (A) according to formula I as defined herein above, it is preferred that the catalyst molding displays a concentration of bridging μ² η²-peroxo species per Ti in the H₂O₂-activated catalyst molding in the range of from 100 to 1,000 mmol/mol, more preferably from 200 to 900 mmol/mol, more preferably from 300 to 800 mmol/mol, more preferably from 400 to 700 mmol/mol, more preferably from 420 to 680 mmol/mol, more preferably from 480 to 620 mmol/mol, and more preferably from 500 to 600 mmol/mol.

Further in the case where the catalyst molding displays a water adsorption (W), a concentration (C) of bridging μ² η²-peroxo species per Ti in the H₂O₂-activated catalyst molding, and an activation factor (A) according to formula I as defined herein above, it is preferred that the concentration (C) of bridging μ² η²-peroxo species per Ti in the H₂O₂-activated catalyst molding as determined by quantitative ¹⁷O NMR spectroscopy is the concentration which is determined at a time point T after having brought the catalyst molding into contact with H₂¹⁷O₂, wherein T is in the range of from 1 to 720 min after having brought the catalyst molding into contact with H₂¹⁷O₂, more preferably from 2 min to 480 min after having brought the catalyst molding into contact with H₂¹⁷O₂, more preferably from 4 to 240 min after having brought the catalyst molding into contact with H₂¹⁷O₂, more preferably from 6 to 120 min after having brought the catalyst molding into contact with H₂¹⁷O₂, more preferably from 8 to 60 min after having brought the catalyst molding into contact with H₂¹⁷O₂, more preferably from 10 to 30 min after having brought the catalyst molding into contact with H₂¹⁷O₂, more preferably from 12 to 20 min after having brought the catalyst molding into contact with H₂¹⁷O₂, and more preferably from 14 to 16 min after having brought the catalyst molding into contact with H₂¹⁷O₂, wherein more preferably T is 15 min after having brought the catalyst molding into contact with H₂¹⁷O₂.

Further in the case where the catalyst molding displays a water adsorption (W), a concentration (C) of bridging μ² η²-peroxo species per Ti in the H₂O₂-activated catalyst molding, and an activation factor (A) according to formula I as defined herein above, it is preferred that the activation factor of the catalyst molding is in the range of from 12 to 70 mmol/mol, more preferably from 14 to 65 mmol/mol, more preferably from 16 to 60 mmol/mol, more preferably from 18 to 55 mmol/mol, more preferably from 19 to 50 mmol/mol, more preferably from 20 to 48 mmol/mol, more preferably from 23 to 46 mmol/mol, more preferably from 26 to 42 mmol/mol, more preferably from 28 to 39 mmol/mol, and more preferably from 30 to 36 mmol/mol. Furthermore, and independently thereof, it is preferred that the catalyst molding displays a concentration of bridging μ² η²-peroxo species per Ti in the H₂O₂-activated catalyst molding in the range of from 100 to 1,000 mmol/mol, wherein the concentration of bridging μ² η²-peroxo species per Ti in the H₂O₂-activated catalyst molding as determined by quantitative ¹⁷O NMR spectroscopy is the concentration which is determined at a time point T after having brought the catalyst molding into contact with H₂¹⁷O₂, wherein T is in the range of from 65 to 175 min. Furthermore, and independently thereof, it is preferred that the catalyst molding displays a concentration of bridging μ² η²-peroxo species per Ti in the H₂O₂-activated catalyst molding as determined by quantitative ¹⁷O NMR spectroscopy, more preferably determined according to Reference Example 1.2, in the range of from 200 to 900 mmol/mol, preferably from 300 to 800 mmol/mol, more preferably from 380 to 750 mmol/mol, more preferably from 420 to 700 mmol/mol, more preferably from 470 to 650 mmol/mol, and more preferably from 500 to 620 mmol/mol. Furthermore, and independently thereof, it is preferred that T is in the range of from 75 to 165 min after having brought the catalyst molding into contact with H₂¹⁷O₂, more preferably from 85 min to 155 min after having brought the catalyst molding into contact with H₂¹⁷O₂, more preferably from 95 to 145 min after having brought the catalyst molding into contact with H₂¹⁷O₂, more preferably from 105 to 135 min after having brought the catalyst molding into contact with H₂¹⁷O₂, more preferably from 112 to 128 min after having brought the catalyst molding into contact with H₂¹⁷O₂, more preferably from 116 to 124 min after having brought the catalyst molding into contact with H₂¹⁷O₂, more preferably from 118 to 122 min after having brought the catalyst molding into contact with H₂¹⁷O₂, and more preferably from 119 to 121 min after having brought the catalyst molding into contact with H₂¹⁷O₂, wherein more preferably T is 120 min after having brought the catalyst molding into contact with H₂¹⁷O₂. Furthermore, and independently thereof, it is preferred that the activation factor of the zeolitic material is in the range of from 12 to 70 mmol/mol, preferably from 14 to 65 mmol/mol, more preferably from 16 to 60 mmol/mol, more preferably from 18 to 55 mmol/mol, more preferably from 19 to 50 mmol/mol, more preferably from 20 to 45 mmol/mol, more preferably from 21 to 40 mmol/mol, more preferably from 22 to 37 mmol/mol, more preferably from 23 to 35 mmol/mol, and more preferably from 24 to 34 mmol/mol.

It is preferred that the catalyst molding has a BET specific surface area in the range of from 200 to 450 m²/g, more preferably in the range of from 220 to 420 m²/g, more preferably in the range of from 240 to 400 m²/g, more preferably in the range of from 250 to 390 m²/g, wherein the BET specific surface area is preferably determined according to Reference Example 1.4.

It is preferred that the UV-vis spectrum of the catalyst molding displays a first absorption band A1 having a maximum in the range of from 200 to 240 nm, wherein the UV-vis spectrum of the catalyst molding preferably displays a second absorption band A2 having a maximum in the range of from 241 to 330 nm, more preferably of from 241 to 320 nm, and more preferably of from 241 to 300 nm.

It is preferred that the catalyst molding shows a selectivity towards the sum of 1-methoxy-2-propanol and 2-methoxy-1-propanol in the range of from 0 to 15%, more preferably in the range of from 0.1 to 9%, more preferably in the range of from 0.1 to 6.5%, preferably determined according to Example 14, more preferably determined according to Example 14 after a runtime in the range of from 22 to 24 h, more preferably determined according to Example 14 after a runtime of 24 h.

It is preferred that the catalyst molding shows a selectivity towards the sum of 1-methoxy-2-propanol and 2-methyoxy-1-propanol in the range of from 0 to 15%, more preferably in the range of from 0.1 to 9%, more preferably in the range of from 0.1 to 6.5%, preferably determined according to Example 14, more preferably determined according to Example 14 when the feed stream reaches full load of hydrogen peroxide for the first time.

It is preferred that the catalyst molding shows a deactivation rate in the range of from 0 to 0.055 K/h, more preferably in the range of from 0.001 to 0.035 K/h, wherein the deactivation rate is determined as described in Example 14.

It is preferred that the catalyst molding is an extrudate or a granule.

It is preferred that the catalyst molding is in the form of a strand or a sphere.

It is preferred that the zeolitic material comprised in the catalyst molding exhibits a water adsorption in the range of from 1 to 6.9 weight-%, more preferably in the range of from 1.25 to 6.8 weight-%, more preferably in the range of from 1.5 to 6.5 weight-%, more preferably in the range of from 1.5 to 6.25 weight-%, more preferably in the range of from 2.0 to 6.0 weight-%, more preferably in the range of from 2.5 to 5.75 weight-%, more preferably in the range of from 3.0 to 5.5 weight-%, more preferably in the range of from 3.25 to 5.25 weight-%, more preferably in the range of from 3.5 to 5.0 weight-%, more preferably in the range of from 3.75 to 4.75 weight-%, more preferably in the range of from 4.0 to 4.5 weight-%, wherein the water adsorption is preferably determined according to Reference Example 1.1. Alternatively, it is particularly preferred that the zeolitic material exhibits a water adsorption in the range of from 3 to 6.5 wt.-%, more preferably from 3.2 to 4.5 wt.-%, more preferably from 3.4 to 4.1 wt.-%.

It is preferred that from 98 to 100 weight-%, more preferably from 99 to 100 weight-%, more preferably from 99.5 to 100 weight-%, more preferably from 99.9 to 100 weight-%, of the zeolitic material comprised in the catalyst molding and having framework type MFI consist of Ti, Si, O, and H.

It is preferred that the zeolitic material comprised in the catalyst molding has a Ti content in the range of from 0.3 to 3.0 weight-%, more preferably in the range of from 0.4 to 2.0 weight-%, more preferably in the range of from 0.5 to 1.5 weight-%, more preferably in the range of from 0.6 to 1.4 weight-%, more preferably in the range of from 0.7 to 1.3 weight-%, more preferably in the range of from 0.8 to 1.2 weight-%, calculated as elemental Ti and based on the weight of the zeolitic material. Alternatively, it is particularly preferred that the zeolitic material has a Ti content in the range of from 0.7 to 2.5 weight-%, preferably of from 1.0 to 1.9 weight-%, more preferably of from 1.1 to 1.3 weight-%.

It is preferred that the zeolitic material comprised in the catalyst molding has a Si content in the range of from 36 to 48 weight-%, more preferably in the range of from 38 to 46 weight-%, more preferably in the range of from 39 to 45 weight-%, calculated as elemental Si and based on the weight of the zeolitic material.

It is preferred that the zeolitic material comprised in the catalyst molding displays a water adsorption (W), preferably determined according to Reference Example 1.1, a concentration (C) of bridging μ² η²-peroxo species per Ti in the H₂O₂-activated zeolitic material, as determined by quantitative ¹⁷O NMR spectroscopy, preferably determined according to Reference Example 1.2, and an activation factor (A) according to formula I, wherein the activation factor is in the range of from 10 to 75 mmol/mol;
wherein in accordance with formula I, the activation factor is the multiplication product of the water adsorption and the concentration of bridging μ² η²-peroxo species per Ti in the H₂O₂-activated zeolitic material:

A=W×C (I).

According to a first alternative, in the case where the zeolitic material comprised in the catalyst molding displays a water adsorption (W), a concentration (C) of bridging μ² η²-peroxo species per Ti in the H₂O₂-activated zeolitic material, and an activation factor (A) according to formula I as disclosed herein above, it is preferred that the zeolitic material comprised in the catalyst molding displays a concentration of bridging μ² η²-peroxo species per Ti in the H₂O₂-activated zeolitic material in the range of from 100 to 1,000 mmol/mol, more preferably from 200 to 900 mmol/mol, more preferably from 300 to 850 mmol/mol, more preferably from 400 to 800 mmol/mol, more preferably from 500 to 750 mmol/mol, more preferably from 600 to 700 mmol/mol, and more preferably from 640 to 680 mmol/mol. Furthermore, and independently thereof, it is preferred that the concentration (C) of bridging μ² η²-peroxo species per Ti in the H₂O₂-activated zeolitic material as determined by quantitative ¹⁷O NMR spectroscopy is the concentration which is determined at a time point T after having brought the zeolitic material into contact with H₂¹⁷O₂, wherein T is in the range of from 1 to 720 min after having brought the zeolitic material into contact with H₂¹⁷O₂, more preferably from 2 min to 480 min after having brought the zeolitic material into contact with H₂¹⁷O₂, more preferably from 4 to 240 min after having brought the zeolitic material into contact with H₂¹⁷O₂, more preferably from 6 to 120 min after having brought the zeolitic material into contact with H₂¹⁷O₂, more preferably from 8 to 60 min after having brought the zeolitic material into contact with H₂¹⁷O₂, more preferably from 10 to 30 min after having brought the zeolitic material into contact with H₂¹⁷O₂, more preferably from 12 to 20 min after having brought the zeolitic material into contact with H₂¹⁷O₂, and more preferably from 14 to 16 min after having brought the zeolitic material into contact with H₂¹⁷O₂, wherein more preferably T is 15 min after having brought the zeolitic material into contact with H₂¹⁷O₂. Furthermore, and independently thereof, it is preferred that the activation factor of the zeolitic material comprised in the catalyst molding is in the range of from 12 to 70 mmol/mol, more preferably from 14 to 65 mmol/mol, more preferably from 16 to 60 mmol/mol, more preferably from 18 to 55 mmol/mol, more preferably from 20 to 50 mmol/mol, more preferably from 22 to 48 mmol/mol, more preferably from 23 to 43 mmol/mol, more preferably from 24 to 39 mmol/mol, more preferably from 25 to 35 mmol/mol, and more preferably from 26 to 32 mmol/mol.

According to a second alternative, in the case where the zeolitic material comprised in the catalyst molding displays a water adsorption (W), a concentration (C) of bridging μ² η²-peroxo species per Ti in the H₂O₂-activated zeolitic material, and an activation factor (A) according to formula I as disclosed herein above, it is preferred that the zeolitic material comprised in the catalyst molding displays a concentration of bridging μ² η²-peroxo species per Ti in the H₂O₂-activated zeolitic material in the range of from 100 to 1,000 mmol/mol, wherein the concentration of bridging μ² η²-peroxo species per Ti in the H₂O₂-activated zeolitic material as determined by quantitative ¹⁷O NMR spectroscopy is the concentration which is determined at a time point T after having brought the zeolitic material into contact with H₂¹⁷O₂, wherein T is in the range of from 65 to 175 min. Furthermore, and independently thereof, it is preferred that the zeolitic material comprised in the catalyst molding displays a concentration of bridging μ² η²-peroxo species per Ti in the H₂O₂-activated zeolitic material as determined by quantitative ¹⁷O NMR spectroscopy, preferably determined according to Reference Example 1.2, in the range of from 200 to 900 mmol/mol, preferably from 300 to 800 mmol/mol, more preferably from 400 to 750 mmol/mol, more preferably from 460 to 700 mmol/mol, more preferably from 510 to 650 mmol/mol, and more preferably from 550 to 610 mmol/mol. Furthermore, and independently thereof, it is preferred that T is in the range of from 75 to 165 min after having brought the zeolitic material into contact with H₂¹⁷O₂, more preferably from 85 min to 155 min after having brought the zeolitic material into contact with H₂¹⁷O₂, more preferably from 95 to 145 min after having brought the zeolitic material into contact with H₂¹⁷O₂, more preferably from 105 to 135 min after having brought the zeolitic material into contact with H₂¹⁷O₂, more preferably from 112 to 128 min after having brought the zeolitic material into contact with H₂¹⁷O₂, more preferably from 116 to 124 min after having brought the zeolitic material into contact with H₂¹⁷O₂, more preferably from 118 to 122 min after having brought the zeolitic material into contact with H₂¹⁷O₂, and more preferably from 119 to 121 min after having brought the zeolitic material into contact with H₂¹⁷O₂, wherein more preferably T is 120 min after having brought the zeolitic material into contact with H₂¹⁷O₂. Furthermore, and independently thereof, it is preferred that the activation factor of the zeolitic material comprised in the catalyst molding is in the range of from 12 to 70 mmol/mol, more preferably from 14 to 65 mmol/mol, more preferably from 16 to 60 mmol/mol, more preferably from 18 to 55 mmol/mol, more preferably from 19 to 50 mmol/mol, more preferably from 20 to 45 mmol/mol, more preferably from 21 to 40 mmol/mol, more preferably from 22 to 35 mmol/mol, more preferably from 23 to 31 mmol/mol, and more preferably from 24 to 28 mmol/mol.

It is preferred that the zeolitic material comprised in the catalyst molding has a molar ratio of Ti atoms comprised in the framework structure of the zeolitic material to the total amount of Ti atoms comprised in the zeolitic material in the range of from 0.5:1 to 1:1, more preferably in the range of from 0.55:1 to 1:1, more preferably in the range of from 0.6:1 to 1:1, preferably determined via X-ray photoelectron spectroscopy (XPS).

It is preferred that the zeolitic material comprised in the catalyst molding has a Na content, calculated as Na₂O, in the range of from 0 to 0.5 weight-%, more preferably of from 0 to 0.2 weight-%, more preferably of from 0 to 0.15 weight-%, more preferably of from 0 to 0.14 weight-%, more preferably of from 0 to 0.1 weight-%, more preferably in the range of from 0 to 0.07 weight-%, more preferably in the range of from 0 to 0.05 weight-%, based on the weight of the zeolitic material.

It is preferred that the zeolitic material comprised in the catalyst molding has a content of one or more of Fe, Co, Ni, and Cu, calculated as Fe₂O₃, Co₂O₃, NiO, and CuO, respectively, in the range of from 0 to 0.1 weight-%, more preferably in the range of from 0 to 0.07 weight-%, more preferably in the range of from 0 to 0.05 weight-%, more preferably in the range of from 0 to 0.02 weight-%, based on the weight of the zeolitic material. Alternatively, it is preferred that the zeolitic material comprised in the catalyst molding has a content of one or more of Fe, Co, Ni, and Cu, calculated as the element, respectively, in the range of from 0 to 0.1 weight-%, more preferably in the range of from 0 to 0.07 weight-%, more preferably in the range of from 0 to 0.05 weight-%, more preferably in the range of from 0 to 0.02 weight-%, based on the weight of the zeolitic material.

It is preferred that the zeolitic material comprised in the catalyst molding has an B content, calculated as B₂O₃, in the range of from 0 to 0.1 weight-%, more preferably in the range of from 0 to 0.01 weight-%, more preferably in the range of from 0 to 0.001 weight-%, based on the weight of the zeolitic material.

It is preferred that the zeolitic material comprised in the catalyst molding has a Ge content, calculated as GeO₂, in the range of from 0 to 0.1 weight-%, more preferably in the range of from 0 to 0.01 weight-%, more preferably in the range of from 0 to 0.001 weight-%, based on the weight of the zeolitic material.

It is preferred that the zeolitic material comprised in the catalyst molding has a C content, calculated as elemental C, in the range of from 0 to 0.1 weight-%, more preferably in the range of from 0 to 0.01 weight-%, more preferably in the range of from 0 to 0.001 weight-%, based on the weight of the zeolitic material.

It is preferred that the zeolitic material comprised in the catalyst molding has a crystallinity in the range of from 50 to 110 weight, more preferably in the range of from 50 to 100 weight-%, more preferably in the range of from 70 to 100 weight-%, more preferably in the range of from 80 to 100 weight-%, wherein the crystallinity is preferably determined according to Reference Example 1.3.

It is preferred that the zeolitic material comprised in the catalyst molding displays a BET specific surface area in the range of from 370 to 520 m²/g, more preferably in the range of from 390 to 500 m²/g, more preferably in the range of from 410 to 480 m²/g, more preferably in the range of from 430 to 460 m²/g, wherein the BET specific surface area is preferably determined according to Reference Example 1.4. Alternatively, it is particularly preferred that the zeolitic material comprised in the catalyst molding displays a BET specific surface area in the range of from 390 to 440 m²/g.

It is preferred that the zeolitic material comprised in the catalyst molding exhibits a type IV nitrogen adsorption/desorption isotherm, wherein the nitrogen adsorption/desorption isotherm is preferably determined according to Reference Example 1.8.

It is preferred that the zeolitic material comprised in the catalyst molding is a TS-1 zeolite.

It is preferred that the zeolitic material comprised in the catalyst molding exhibits a propylene oxide activity of at least 2.0 weight-%, preferably in the range of from 3.0 to 15.0 weight-%, more preferably in the range of from 5.0 to 14.0 weight-%, more preferably in the range of from 9.0 to 13.0 weight-%, preferably determined as described in Reference Example 1.5.

It is preferred that the catalyst molding has a bulk density of equal to or greater than 320 g/l, more preferably equal to or greater than 330 g/l, more preferably equal to or greater than 350 g/l, more preferably in the range of from 350 to 600 g/l, more preferably in the range of from 350 to 500 g/l, more preferably in the range of from 360 to 400 g/l, more preferably in the range of from 370 to 400 g/l, more preferably in the range of from 370 to 390 g/l, wherein the bulk density is preferably determined according to Reference Example 1.11.

It is preferred that the catalyst molding exhibits a propylene oxide activity of at least 2.0 weight-%, more preferably in the range of from 3.0 to 15.0 weight-%, more preferably in the range of from 3.5 to 10.0 weight-%, more preferably in the range of from 4.0 to 7.5 weight-%, more preferably in the range of from 4.5 to 6.5 weight-%, more preferably in the range of from 5.5 to 6.3 weight-%, preferably determined as described in Reference Example 1.5.

It is preferred that the catalyst molding exhibits a k-80 test value of less than 0.20 h⁻¹, wherein preferably the catalyst molding exhibits a k-80 test value in the range of from 0.01 to 0.19 h⁻¹, more preferably of from 0.02 to 0.15 h⁻¹, more preferably of from 0.03 to 0.10 h⁻¹, more preferably of from 0.04 to 0.06 h⁻¹, preferably determined as described in Reference Example 1.6.

Further, the present invention relates to a reactor comprising a plurality of catalyst moldings, each of the catalyst moldings independently from one another being in accordance with the catalyst molding according to any one of the particular and preferred embodiments disclosed herein, wherein from 42 to 100% of the plurality of catalyst moldings has an aspect ratio in the range of from greater than 1.5 to smaller than 6.1, preferably in the range of from greater than 1.5 to 6.0, more preferably in the range of from 1.8 to smaller than 6.1, and more preferably within the range of from 1.8 to 6.0, wherein the aspect ratio is preferably determined according to Reference Example 1.12.

It is preferred that from 15 to 25%, more preferably from 18 to 22%, of the plurality of catalyst moldings has an aspect ratio in the range of from 1.4 to smaller than 1.8.

It is preferred that from 37 to 47%, more preferably from 40 to 44%, of the plurality of catalyst moldings has an aspect ratio in the range of from 1.8 to smaller than 2.2.

It is preferred that from 19 to 29%, more preferably from 22 to 26%, of the plurality of catalyst moldings has an aspect ratio in the range of from 2.2 to smaller than 2.7.

It is preferred that from 5 to 15%, more preferably from 8 to 12%, of the plurality of catalyst moldings has an aspect ratio in the range of from 2.7 to smaller than 3.2.

It is preferred that from 1 to 11%, more preferably from 4 to 8%, of the plurality of catalyst moldings has an aspect ratio in the range of from 3.6 to smaller than 4.1.

It is preferred that from 0 to 5%, more preferably from 0 to 2%, of the plurality of catalyst moldings has an aspect ratio in the range of from 3.6 to smaller than 6.1, preferably in the range of from 3.6 to smaller than 4.6.

Alternatively, it is preferred that from 1 to 22%, more preferably from 2 to 20%, of the plurality of catalyst moldings has an aspect ratio in the range of from 1.0 to 1.5.

Furthermore, it is alternatively preferred that from 10 to 46%, more preferably from 12 to 44%, of the plurality of catalyst moldings has an aspect ratio in the range of from greater than 1.5 to 2.0.

Furthermore, it is alternatively preferred that from 9 to 31%, more preferably from 13 to 27%, more preferably from 15 to 25%, of the plurality of catalyst moldings has an aspect ratio in the range of from greater than 2.0 to 2.5.

Furthermore, it is alternatively preferred that from 7 to 27%, more preferably from 9 to 25%, of the plurality of catalyst moldings has an aspect ratio in the range of from greater than 2.5 to 3.0.

Furthermore, it is alternatively preferred that from 1 to 24%, more preferably from 3 to 22%, of the plurality of catalyst moldings has an aspect ratio in the range of from greater than 3.0 to 3.5.

Furthermore, it is alternatively preferred that from 0 to 17, more preferably from 2 to 15%, of the plurality of catalyst moldings has an aspect ratio in the range of from greater than 3.5 to 4.0.

Furthermore, it is alternatively preferred that from 0 to 10, more preferably from 1 to 8%, of the plurality of catalyst moldings has an aspect ratio in the range of from greater than 4.0 to 4.5.

Furthermore, it is alternatively preferred that from 0 to 16, more preferably from 1 to 14%, of the plurality of catalyst moldings has an aspect ratio in the range of from greater than 4.5 to 5.0, preferably in the range of from greater than 4.5 to 6.0.

Further, the present invention relates to a process for preparing a catalyst molding according to any one of the particular and preferred embodiments disclosed herein, the process comprising
(i) preparing a mixture comprising one or more binder precursors and a zeolitic material having framework type MFI, preferably a zeolitic material as defined in any one of the particular and preferred embodiments disclosed herein;
(ii) shaping the mixture obtained from (i) to a catalyst molding precursor, obtaining a precursor of the catalyst molding;
(iii) optionally preparing a mixture comprising the precursor of the catalyst molding obtained from (ii) and water, and subjecting the mixture to a water treatment under hydrothermal conditions, obtaining a water-treated precursor of the catalyst molding;
(iv) calcining the precursor of the catalyst molding obtained from (ii) or the water-treated precursor of the catalyst molding obtained from (iii) in a gas atmosphere, wherein the gas atmosphere preferably comprises, more preferably consists of, one or more of nitrogen and oxygen, preferably air, obtaining the catalyst molding.

It is preferred that the one or more binder precursors are selected from the group consisting of a silica sol, a colloidal silica, a wet process silica, a dry process silica, and a mixture of two or more thereof, wherein the one or more binder precursors more preferably comprise, preferably consist of, a colloidal silica.

It is preferred that the one or more binder precursors comprise Si, wherein in the mixture according to (i), the weight ratio of zeolitic material, relative to Si comprised in the one or more binder precursors, calculated as SiO₂, is preferably in the range of from 1.5:1 to 10:1, more preferably of from 1.8:1 to 6:1, more preferably of from 2:1 to 5.6:1, more preferably of from 2.3:1 to 4:1, more preferably of from 2.5:1 to 3.5:1, more preferably of from 2.9:1 to 3.1:1. Alternatively, it is particularly preferred that the weight ratio of zeolitic material, relative to Si comprised in the one or more binder precursors, calculated as SiO₂, is preferably in the range of from 2:1 to 10:1, more preferably in the range of from 5:1 to 7.5:1, more preferably in the range of from 6.0:1 to 6.5:1. As a further alternative, it is particularly preferred that the weight ratio of zeolitic material, relative to Si comprised in the one or more binder precursors, calculated as SiO₂, is preferably in the range of from 2:1 to 10:1, more preferably in the range of from 4:1 to 6:1, more preferably in the range of from 4.8:1 to 5.0:1.

It is preferred that the mixture prepared according to (i) further comprises one or more agents, wherein the agents comprise, preferably consist of, pore forming agents, preferably mesopore forming agents, lubricants, and plasticizers.

In the case where the mixture prepared according to (i) further comprises one or more agents, it is preferred that the one or more agents comprise, preferably consist of, one or more of water, alcohols, organic polymers, carbohydrates, graphite, plant additives, and mixtures of two or more thereof, preferably one or more of water, polymeric vinyl compounds, polysaccharides, polyalkylene oxides, polystyrenes, polyacrylates, polymethacrylates, polyolefins, polyamides, polyesters, and mixtures of two or more thereof, more preferably one or more of water, polymeric vinyl compounds, celluloses, cellulose derivatives, polymannoses, polymannose derivatives, starches, Sesbania cannabina leaf powder, Sesbania cannabina gum powder, polyalkylene oxides, polystyrenes, polyacrylates, polymethacrylates, polyolefins, polyamides, polyesters, and mixtures of two or more thereof, more preferably one or more of water, polymeric vinyl compounds, polyalkylene oxides, polyacrylates, polyolefins, polystyrenes, polyamides, polyesters, celluloses, cellulose derivatives, polymannoses, polymannose derivatives, Sesbania cannabina leaf powder, Sesbania cannabina gum powder, and mixtures of two or more thereof, more preferably one or more of water, polystyrene, C₂ to C₃ polyalkylene oxides, polymeric vinyl compounds, cellulose, hydroxymethylcellulose, methoxy cellulose, ethoxy cellulose, polymannose at least partially derivatised with galactose, Sesbania cannabina leaf powder, Sesbania cannabina gum powder, and mixtures of two or more thereof, more preferably one or more of water, polystyrene, polyethylene oxide, a polyvinylacetate, a cellulose, a galactomannan, Sesbania cannabina leaf powder, Sesbania cannabina gum powder, and mixtures of two or more thereof, more preferably one or more of water, polyethylene oxide, a polyvinylacetate, a cellulose, a galactomannan, Sesbania cannabina leaf powder, Sesbania cannabina gum powder, and mixtures of two or more thereof, wherein the one or more agents more preferably comprise, more preferably consist of, one or more of water, a polyvinylacetate, a cellulose, a galactomannan, Sesbania cannabina leaf powder, Sesbania cannabina gum powder, and mixtures of two or more thereof.

Further in the case where the mixture prepared according to (i) further comprises one or more agents, it is preferred that in the mixture prepared according to (i), the weight ratio of zeolitic material, relative to the one or more agents is in the range of from 1:1 to 99:1, more preferably in the range of from 1.3:1 to 95:1, more preferably in the range of from 1.5:1 to 90:1, more preferably in the range of from 1.8:1 to 70:1, more preferably in the range of from 2.0:1 to 50:1, more preferably in the range of from 2.3:1 to 30:1, more preferably in the range of from 2.5:1 to 15:1, more preferably in the range of from 2.8:1 to 8:1, more preferably in the range of from 3:1 to 4:1. Alternatively, it is particularly preferred that the weight ratio of zeolitic material, relative to the one or more agents is in the range of from 1:1 to 5:1, more preferably in the range of from 2.5:1 to 4:1, more preferably in the range of from 1.8:1 to 2.0:1.

It is preferred that the mixture prepared according to (i) comprises from 0 to 0.1 weight-%, more preferably from 0 to 0.01 weight-%, more preferably from 0 to 0.001 weight-%, of ammonia, preferably of a hydrolyzing agent, based on the weight of the mixture.

It is preferred that the mixture prepared according to (i) is mixed in a kneader or in a mix-muller.

It is preferred that, in (ii), the mixture is shaped to a catalyst molding precursor having an aspect ratio D1:D2 in the range of from 1.4:1 to 6.1:1, more preferably in the range of from 1.45:1 to 5.6:1, more preferably in the range of from 1.5:1 to 5.1:1, more preferably in the range of from 1.55:1 to 4.6:1, more preferably in the range of from 1.6:1 to 4.1:1, more preferably in the range of from 1.65:1 to 3.6:1, more preferably in the range of from 1.7:1 to 3.2:1, more preferably in the range of from 1.75:1 to 2.7:1, more preferably in the range of from 1.8:1 to 2.2:1, wherein the aspect ratio is preferably determined according to Reference Example 1.12.

It is preferred that, in (ii), the mixture is shaped to a catalyst molding precursor, wherein D1 is in the range of from 0.1 to 10 mm, more preferably in the range of from 1.2 to 7.0 mm, more preferably in the range of from 2.2 to 5.0 mm, more preferably in the range of from 3.0 to 4.0 mm, more preferably in the range of from 3.4 to 3.8 mm.

It is preferred that, in (ii), the mixture is shaped to a catalyst molding precursor, wherein D2 is in the range of from 0.05 to 5 mm, more preferably in the range of from 0.6 to 3.5 mm, more preferably in the range of from 1.1 to 2.5 mm, more preferably in the range of from 1.5 to 2.0 mm, more preferably in the range of from 1.7 to 1.9.

It is preferred that, in (ii), the mixture is shaped to a catalyst molding precursor having a cross-sectional profile, wherein the cross-sectional profile is circular, hexagonal, rectangular, quadratic, triangular, oval, a star-shaped polygon, or cloverleaf-shaped, preferably circular, hexagonal, rectangular, quadratic, triangular, oval, a star-shaped polygon having 3, 4, 5, 6, 7, or 8 tips, a trilobe or a quadrilobe, more preferably circular, hexagonal, rectangular, quadratic, triangular, oval, a star-shaped polygon having 3 or 4 tips, a trilobe or a quadrilobe.

In the case where the mixture is shaped in (ii) to a catalyst molding precursor having a cross-sectional profile, wherein the cross-sectional profile is circular, hexagonal, rectangular, quadratic, triangular, oval, a star-shaped polygon, or cloverleaf-shaped, it is preferred that the cross-sectional profile is a star-shaped polygon having 3 tips, wherein the tips are rounded and wherein the corners are rounded.

It is preferred that, in (ii), the mixture is shaped to a catalyst molding precursor, wherein P3 and P1 have a cutting angle in the range of from 60° to 90°, more preferably in the range of from 70° to 90°, more preferably in the range of from 80° to 90°, more preferably in the range of from 85° to 90°, more preferably in the range of from 89° to 90°, wherein P3 more preferably is substantially perpendicular to P1.

It is preferred that, in (ii), the mixture is shaped to a catalyst molding precursor having the shape of an extrudate, said shape having a length in the conceived or actual direction of extrusion, and said shape having a fixed cross-sectional profile perpendicular to said conceived or actual direction of extrusion.

In the case where the mixture is shaped in (ii) to a catalyst molding precursor having the shape of an extrudate, said shape having a length in the conceived or actual direction of extrusion, and said shape having a fixed cross-sectional profile perpendicular to said conceived or actual direction of extrusion, it is preferred that the catalyst molding having the shape of an extrudate has a circular cross-sectional profile with a diameter D, the diameter D corresponding to D2.

It is preferred that, in (ii), shaping comprises extruding the mixture, optionally by piston press or extruder, preferably by screw extruder, more preferably by single or twin screw extruder, more preferably by single screw extruder.

It is preferred that shaping according to (ii) further comprises drying the precursor of the molding in a gas atmosphere, wherein the gas atmosphere preferably comprises, more preferably consists of, one or more of an inert gas, preferably nitrogen, and oxygen. Furthermore, and independently thereof, it is preferred that drying is carried out at a temperature of the gas atmosphere in the range of from 80 to 160 °C, more preferably in the range of from 100 to 140 °C, more preferably in the range of from 110 to 130 °C.

It is preferred that shaping according to (ii) further comprises, preferably after drying the precursor of the molding according to embodiment 100 or 101, calcining the precursor of the molding in a gas atmosphere, wherein the gas atmosphere preferably comprises, more preferably consists of, one or more of an inert gas, preferably nitrogen, and oxygen. If shaping according to (ii) further comprises calcining the precursor of the molding in a gas atmosphere, it is preferred that calcining is carried out at a temperature of the gas atmosphere in the range of from 350 to 700 °C, preferably in the range of from 400 to 490 °C, preferably in the range of from 420 to 470 °C, more preferably in the range of from 440 to 460 °C.

It is preferred that the water treatment according to (iii) treatment is performed with a water containing solvent system and/or with an aqueous solution, wherein more preferably the hydrothermal treatment is performed with deionized water.

It is preferred that the water treatment according to (iii) comprises a temperature of the mixture in the range of from 100 to 200 °C, more preferably in the range of from 125 to 175 °C, more preferably in the range of from 130 to 160 °C, more preferably in the range of from 135 to 155 °C more preferably in the range of from 140 to 150 °C.

It is preferred that the water treatment according to (iii) is carried out under autogenous pressure, more preferably in an autoclave.

It is preferred that the water treatment according to (iii) is carried out for 6 to 15 h, more preferably for 6 to 10 h, more preferably for 7 to 9 h, more preferably for 7.5 to 8.5 h.

It is preferred that in the mixture prepared in (iii), the weight ratio of the precursor of the catalyst molding obtained from (ii) relative to the water is in the range of from 1:5 to 1:25, more preferably in the range of from 1:10 to 1:20, more preferably in the range of from 1:13 to 1:17.

It is preferred that, after (iii) and prior to (iv), the water-treated precursor of the molding is separated from the mixture obtained from (iii), wherein separating more preferably comprises subjecting the mixture obtained from (iii) to filtration or centrifugation, wherein more preferably, separating further comprises washing the water-treated precursor of the molding at least once with a liquid solvent system, wherein the liquid solvent system preferably comprises one or more of water, an alcohol, and a mixture of two or more thereof, wherein the water-treated precursor of the molding is more preferably washed with water.

It is preferred that, after (iii) and prior to (iv), the preferably separated water-treated precursor of the molding is dried in a gas atmosphere, wherein drying is preferably carried out at a temperature of the gas atmosphere in the range of from 80 to 160 °C, more preferably in the range of from 100 to 140 °C, more preferably in the range of from 110 to 130 °C, wherein the gas atmosphere preferably comprises air.

It is preferred that calcining according to (iv) is carried out at a temperature of the gas atmosphere in the range of from 350 to 700 °C, more preferably in the range of from 400 to 490 °C, preferably in the range of from 420 to 470 °C, more preferably in the range of from 440 to 460 °C.

It is preferred that the zeolitic material having framework type MFI in the mixture prepared according to (i) is prepared according to a process comprising
(a) providing a zeolitic material having framework type MFI;
(b) preparing an aqueous mixture comprising the zeolitic material provided in (i) and a structure directing agent, wherein the structure directing agent preferably comprises, more preferably consists of, tetrapropylammonium hydroxide and/or tetrapropylammonium bromide, preferably tetrapropylammonium hydroxide;
(c) subjecting the mixture obtained from (b) to hydrothermal conditions under autogenous pressure, preferably in an autoclave, obtaining a suspension comprising a precursor of the zeolitic material having framework type MFI as defined in any one of the embodiments disclosed herein, and separating said precursor from the suspension;
(d) optionally calcining the precursor obtained from (c) in a gas atmosphere, wherein the gas atmosphere preferably comprises, more preferably consists of, air;
(e) optionally subjecting the precursor obtained from (c) or (d) to an acid treatment;
(f) optionally drying the acid-treated precursor obtained from (e) in a gas atmosphere, wherein the gas atmosphere preferably comprises, more preferably consists of, air;
(g) calcining the precursor obtained from (c), (e), or (f) in a gas atmosphere, wherein the gas atmosphere preferably comprises, more preferably consists of, air, obtaining the zeolitic material having framework type MFI according to any one of the embodiments disclosed herein.

It is preferred that from 98 to 100 weight-%, more preferably from 99 to 100 weight-%, more preferably from 99.5 to 100 weight-%, more preferably from 99.9 to 100 weight-%, of the zeolitic material having framework type MFI provided in (a) consist of Ti, Si, O, and H.

It is preferred that the zeolitic material provided in (a) exhibits a type I nitrogen adsorption/desorption isotherm, wherein the nitrogen adsorption/desorption isotherm is preferably determined according to Reference Example 1.8.

It is preferred that the zeolitic material provided in (a) has a Ti content in the range of from 0.3 to 3.0 weight-%, more preferably in the range of from 0.4 to 2.0 weight-%, more preferably in the range of from 0.5 to 1.5 weight-%, more preferably in the range of from 0.6 to 1.4 weight-%, more preferably in the range of from 0.7 to 1.3 weight-%, more preferably in the range of from 0.8 to 1.2 weight-%, calculated as elemental Ti and based on the weight of the zeolitic material provided in (a). Alternatively, it is particularly preferred that the zeolitic material has a Ti content in the range of from 0.7 to 2.5 weight-%, more preferably of from 1.0 to 1.9 weight-%, more preferably of from 1.1 to 1.3 weight-%.

It is preferred that the aqueous mixture prepared in (b) comprises the zeolitic material provided in (a) and the structure directing agent in a weight ratio in the range of from 0.25:1 to 5:1, more preferably in the range of from 0.75:1 to 1.5:1.

It is preferred that the aqueous mixture prepared in (b) comprises the zeolitic material provided in (a) and water in a weight ratio in the range of from 0.01:1 to 0.50:1, more preferably in the range of from 0.10:1 to 0.20:1.

Optionally, the aqueous mixture prepared in (b) further comprises NH₃ and/or a source of NH₃, wherein preferably the source of NH₃ comprises, preferably consists of, urea.

It is preferred that subjecting the aqueous mixture obtained from (b) according to (c) to hydrothermal conditions comprises heating the mixture to a temperature in the range of from 140 to 200 °C, more preferably in the range of from 160 to 180 °C.

It is preferred that the aqueous mixture obtained from (b) is subjected according to (c) to hydrothermal conditions for a duration in the range of from 60 to 110 h, more preferably in the range of from 75 to 95 h.

It is preferred that the precursor obtained from (c) is calcined according to (d) at a temperature in the range of from 450 to 530 °C, more preferably in the range of from 470 to 510 °C.

It is preferred that the precursor obtained from (c) is calcined according to (d) for a duration in the range of from 2 to 8 h, more preferably in the range of from 4 to 6 h.

It is preferred that the acid treatment according to (e) comprises bringing the precursor obtained from (c) or (d) in contact with an aqueous mixture comprising an acid, wherein the acid preferably comprises nitric acid, wherein the aqueous mixture more preferably comprises from 5 to 15 weight-% of nitric acid, based on the total weight of aqueous mixture.

It is preferred that the acid-treated precursor obtained from (e) is dried in (f) at a temperature in the range of from 100 to 140 °C, more preferably in the range of from 110 to 130 °C.

It is preferred that the precursor obtained from (c), (e), or (f) is calcined according to (g) at a temperature in the range of from 450 to 530 °C, more preferably in the range of from 470 to 510 °C.

It is preferred that the precursor obtained from (c), (e), or (f) is calcined according to (g) atmosphere for a duration in the range of from 2 to 8 h, more preferably in the range of from 4 to 6 h.

Yet further, the present invention relates to a catalyst molding obtainable or obtained according to the process of any one of the particular and preferred embodiments disclosed herein.

Yet further, the present invention relates to a process for the activation of hydrogen peroxide comprising:
(1) providing a reactor comprising a catalyst molding according to any one of the particular and preferred embodiments disclosed herein or a reactor according to any one of the particular and preferred embodiments disclosed herein;
(2) contacting the catalyst molding provided in (1) or the plurality of catalyst moldings comprised in the reactor provided in (1) with hydrogen peroxide.

It is preferred that contacting in (2) is conducted at a temperature in the range of from 10 to 100 °C, more preferably from 20 to 80 °C, more preferably from 25 to 75 °C, more preferably from 30 to 65 °C.

It is preferred that contacting in (2) is conducted at a pressure in the range of from 5 to 100 bar, more preferably from 10 to 50 bar, more preferably from 14 to 32 bar, more preferably from 15 to 25 bar, wherein the pressure is defined as the pressure at the exit of the reactor.

It is preferred that in (2) hydrogen peroxide is comprised in a liquid feed stream which is fed into the reactor, wherein the liquid feed stream further comprises one or more unsaturated organic compounds, more preferably one or more olefins, more preferably one or more C₂ to C₅ alkenes, more preferably one or more C₂ to C₄ alkenes, more preferably one or more C₂ or C₃ alkenes, more preferably propylene.

In the case where in (2) hydrogen peroxide is comprised in a liquid feed stream which is fed into the reactor, it is preferred that the liquid feed stream further comprises a solvent system, wherein the solvent system comprises one or more solvents, wherein more preferably the solvent system comprises one or more hydrophilic solvents, the hydrophilic solvents preferably being selected from the group consisting of polar solvents, more preferably from the group consisting of polar protic solvents, wherein more preferably the solvent system comprises one or more polar protic solvents selected from the group consisting of water, alcohols, and mixtures of two or more thereof, more preferably from the group consisting of water, C₁ to C₅ alcohols, and mixtures of two or more thereof, more preferably from the group consisting of water, C₁ to C₄ alcohols, and mixtures of two or more thereof, more preferably from the group consisting of water, C₁ to C₃ alcohols, and mixtures of two or more thereof, more preferably from the group consisting of water, methanol, ethanol, propanol, and mixtures of two or more thereof, more preferably from the group consisting of water, methanol, and mixtures thereof, wherein more preferably the solvent system comprises water, preferably water and methanol, wherein more preferably the solvent system consists of water and methanol.

Further in the case where in (2) hydrogen peroxide is comprised in a liquid feed stream which is fed into the reactor, it is preferred that the liquid feed stream further comprises a potassium-containing compound, preferably a potassium salt, more preferably one or more of dipotassium hydrogen phosphate and a potassium salt of etidronic acid.

Further in the case where in (2) hydrogen peroxide is comprised in a liquid feed stream which is fed into the reactor, it is preferred that the liquid feed stream comprises hydrogen peroxide at a concentration in the range of from 1 to 75 weight-%, more preferably from 3 to 50 weight-%, 5 to 30 weight-%, more preferably from 7 to 25 weight-%, more preferably from 8 to 20 weight-%, more preferably from 9 to 15 weight-%, more preferably from 10 to 12 weight-%, based on the total weight of the liquid feed stream.

Further in the case where in (2) hydrogen peroxide is comprised in a liquid feed stream which is fed into the reactor, it is preferred that the liquid feed stream fed into the reactor in (2) has a temperature in the range of from 0 to 60 °C, more preferably from 25 to 50 °C.

Further in the case where in (2) hydrogen peroxide is comprised in a liquid feed stream which is fed into the reactor, it is preferred that the liquid feed stream fed into the reactor in (2) is at a pressure in the range of from 5 to 100 bar, more preferably from 10 to 50 bar, more preferably from 15 to 25 bar.

Further in the case where in (2) hydrogen peroxide is comprised in a liquid feed stream which is fed into the reactor, it is preferred that the loading of the catalyst molding in the reactor in (1) is in the range of from 0.05 to 5 h⁻¹, more preferably from 0.1 to 3 h⁻¹, more preferably from 0.2 to 1 h⁻¹, more preferably from 0.200 to 0.5 h⁻¹, more preferably from 0.210 to 0.25 h⁻¹, more preferably from 0.215 to 0.240 h⁻¹, more preferably from 0.220 to 0.235 h⁻¹, more preferably from 0.225 to 0.230 h⁻¹, wherein the loading of the catalyst molding is defined as the ratio of the mass flow rate in kg/h of hydrogen peroxide contained in the liquid feed stream divided by the amount in kg of the catalyst molding comprised in the reactor in (1).

It is preferred that the process for the activation of hydrogen peroxide further comprises
(3) removing an effluent stream from the reactor, the effluent stream comprising an oxidized organic compound, and preferably comprising an epoxidized organic compound, more preferably an alkylene oxide, more preferably an alkylene oxide selected from C₂ to C₅ alkylene oxides, more preferably from C₂ to C₄ alkylene oxides, more preferably from C₂ or C₃ alkylene oxides, more preferably from C₃ alkylene oxides, wherein more preferably the effluent stream comprises propylene oxide.

Yet further, the present invention relates to a use of the catalyst molding according to any one of the particular and preferred embodiments disclosed herein, as a catalyst and/or catalyst component, or a reactor according to any one of the particular and preferred embodiments disclosed herein, in a reaction involving one or more of C—O bond formation, C—C bond formation and C—C bond conversion, and preferably as a catalyst and/or catalyst component in an isomerization reaction, in an ammoximation reaction, in an amination reaction, in a hydrocracking reaction, in an alkylation reaction, in an acylation reaction, in a reaction for the conversion of alkanes to olefins, or in a reaction for the conversion of one or more oxygenates to olefins and/or aromatics, in a reaction for the synthesis of hydrogen peroxide, in an aldol condensation reaction, in a reaction for the isomerization of epoxides, in a transesterification reaction, in a hydroxylation reaction, in a Baeyer-Villiger-type oxidation reaction, in a Dakin-type reaction, or in an epoxidation reaction, preferably as a catalyst and/or catalyst component in a hydroxylation reaction, in a Baeyer-Villiger-type oxidation reaction, in a Dakin-type reaction, or in a reaction for the epoxidation of olefins, more preferably in a reaction for the epoxidation of olefins, more preferably in a reaction for the epoxidation of C₂ to C₅ alkenes, more preferably in a reaction for the epoxidation of C₂ to C₄ alkenes, in a reaction for the epoxidation of C₂ or C₃ alkenes, more preferably for the epoxidation of C₃ alkenes, and more preferably as a catalyst or catalyst component for the conversion of propylene to propylene oxide.

Yet further, the present invention relates to a process for preparing an olefin oxide comprising
(A) providing olefin, hydrogen peroxide, water and organic solvent and optionally an additive into an epoxidation zone comprising the catalyst molding according to any one of the particular and preferred embodiments disclosed herein, as a catalyst and/or catalyst component, or a reactor according to any one of the particular and preferred embodiments disclosed herein, obtaining a reaction mixture comprising olefin, hydrogen peroxide, water and organic solvent;
(B) subjecting the reaction mixture obtained from (A) to epoxidation reaction conditions in the epoxidation zone, thereby obtaining a mixture comprising olefin oxide, water and organic solvent;
(C) removing an effluent stream from the epoxidation zone, comprising olefin oxide, water and organic solvent.

It is preferred that the olefin is a C₂-C₁₀ alkene, more preferably a C₂-C₅ alkene, more preferably a C₂-C₄ alkene, more preferably ethylene or propylene, more preferably propylene; and/or, preferably and,
wherein the organic solvent is an organic epoxidation solvent, wherein preferably the organic solvent is selected from the group consisting of C₁ to C₅ mono alcohol, C₁ to C₅ ether, C₁ to C₅ nitrile, and mixtures of two or more thereof, more preferably from the group consisting of tert-butanol, methanol, acetonitrile, methyl tert-butyl ether (MTBE), and mixtures of two or more thereof;
and/or, preferably and,
wherein the reaction mixture obtained in (A) preferably comprises the additive, wherein the additive is more preferably selected from the group consisting of potassium salt of an inorganic acid, potassium salt of an organic acid, ammonia, ammonium salt of an inorganic acid, ammonium salt of an organic acid and mixtures of two or more thereof; wherein the additive preferably comprises a potassium-containing compound, preferably a potassium salt, more preferably one or more of dipotassium hydrogen phosphate and dipotassium etidronate (K₂HEDP). According to preferred embodiments of the invention, the additive is provided in (A) as an aqueous solution, wherein the aqueous solution may further comprise the hydrogen peroxide.

The present invention is further illustrated by the following set of embodiments and combinations of embodiments resulting from the dependencies and back-references as indicated. In particular, it is noted that in each instance where a range of embodiments is mentioned, for example in the context of a term such as “The catalyst molding of any one of embodiments 1 to 4”, every embodiment in this range is meant to be explicitly disclosed for the skilled person, i.e. the wording of this term is to be understood by the skilled person as being synonymous to “The catalyst molding of any one of embodiments 1, 2, 3, and 4”. Further, it is explicitly noted that the following set of embodiments is not the set of claims determining the extent of protection but represents a suitably structured part of the description directed to general and preferred aspects of the present invention.
1. A catalyst molding comprising a zeolitic material having framework type MFI,
   wherein the catalyst molding has an average crush strength in the range of from 3 to 30 N, wherein the crush strength is preferably determined according to Reference Example 1.7,
   wherein the catalyst molding has an aspect ratio D1:D2,
   wherein D1 stands for the largest distance separating a pair of parallel planes P1 and P2 when the catalyst molding is located in-between said parallel planes without transgressing them, and the catalyst molding is in contact with each of the respective planes in at least one point, and wherein D2 stands for the shortest distance separating a pair of parallel planes P3 and P4 when the catalyst molding is located in-between said parallel planes without transgressing them, and the catalyst molding is in contact with each of the respective planes in at least one point, wherein the aspect ratio D1:D2 is equal to or greater than 1:1, wherein the aspect ratio is preferably determined according to Reference Example 1.12.
2. The catalyst molding of embodiment 1, wherein the aspect ratio D1:D2 is in the range of from 1.4:1 to 6.1:1, preferably in the range of from 1.45:1 to 5.6:1, more preferably in the range of from 1.5:1 to 5.1:1, more preferably in the range of from 1.55:1 to 4.6:1, more preferably in the range of from 1.6:1 to 4.1:1, more preferably in the range of from 1.65:1 to 3.6:1, more preferably in the range of from 1.7:1 to 3.2:1, more preferably in the range of from 1.75:1 to 2.7:1, more preferably in the range of from 1.8:1 to 2.2:1, wherein the aspect ratio is preferably determined according to Reference Example 1.12.
3. The catalyst molding of embodiment 1 or 2, wherein D1 is in the range of from 0.1 to 10 mm, more preferably in the range of from 1.2 to 7.0 mm, more preferably in the range of from 2.2 to 5.0 mm, more preferably in the range of from 3.0 to 4.0 mm, more preferably in the range of from 3.4 to 3.8 mm.
4. The catalyst molding of any one of embodiments 1 to 3, wherein D2 is in the range of from 0.05 to 5 mm, preferably in the range of from 0.6 to 3.5 mm, more preferably in the range of from 1.1 to 2.5 mm, more preferably in the range of from 1.5 to 2.0 mm, more preferably in the range of from 1.7 to 1.9 mm.
5. The catalyst molding of any one of embodiments 1 to 4, wherein the catalyst molding has a cross-sectional profile, wherein the cross-sectional profile is circular, hexagonal, rectangular, quadratic, triangular, oval, a star-shaped polygon, or cloverleaf-shaped, preferably circular, hexagonal, rectangular, quadratic, triangular, oval, a star-shaped polygon having 3, 4, 5, 6, 7, or 8 tips, a trilobe or a quadrilobe, more preferably circular, hexagonal, rectangular, quadratic, triangular, oval, a star-shaped polygon having 3 or 4 tips, a trilobe or a quadrilobe.
6. The catalyst molding of embodiment 5, wherein the cross-sectional profile is a starshaped polygon having 3 tips, wherein the tips are rounded and wherein the corners are rounded.
7. The catalyst molding of any one of embodiments 1 to 6, wherein P3 and P1 form an angle in the range of from 60° to 90°, preferably in the range of from 70° to 90°, more preferably in the range of from 80° to 90°, more preferably in the range of from 85° to 90°, more preferably in the range of from 89° to 90°.
8. The catalyst molding of any one of embodiments 1 to 7, wherein the catalyst molding has the shape of an extrudate, said shape having a length in the conceived or actual direction of extrusion, and said shape having a fixed cross-sectional profile perpendicular to said conceived or actual direction of extrusion.
9. The catalyst molding of embodiment 8, wherein the catalyst molding having the shape of an extrudate has a circular cross-sectional profile with a diameter D, the diameter D corresponding to D2.
10. The catalyst molding of any one of embodiments 1 to 9, having a total pore volume in the range of from 0.50 to 1.2 ml/g, preferably in the range of from 0.60 to 1.2 ml/g, more preferably in the range of from 0.65 to 1.1 ml/g, more preferably in the range of from 0.70 to 1.0 ml/g.
11. The catalyst molding of any one of embodiments 1 to 10, wherein the catalyst molding exhibits an average crush strength in the range of from 3 to 30 N, preferably in the range of from 4 to 25 N, more preferably in the range of from 5 to 20 N, more preferably in the range of from 5 to 15 N, wherein the average crush strength is preferably determined according to Reference Example 1.7.
12. The catalyst molding of any one of embodiments 1 to 11, further comprising one or more oxidic binders, wherein the one or more oxidic binders are preferably selected from the group consisting of inorganic binders, wherein the one or more binders more preferably comprise one or more sources of a metal oxide and/or of a metalloid oxide, more preferably one or more sources of a metal oxide and/or of a metalloid oxide selected from the group consisting of silica, alumina, titania, zirconia, lanthana, magnesia, and mixtures and/or mixed oxides of two or more thereof, more preferably from the group consisting of silica, alumina, titania, zirconia, magnesia, silica-alumina mixed oxides, silica-titania mixed oxides, silica-zirconia mixed oxides, silica-lanthana mixed oxides, silica-zirconia-lanthana mixed oxides, alumina-titania mixed oxides, alumina-zirconia mixed oxides, alumina-lanthana mixed oxides, alumina-zirconia-lanthana mixed oxides, titania-zirconia mixed oxides, and mixtures and/or mixed oxides of two or more thereof, more preferably from the group consisting of silica, alumina, silica-alumina mixed oxides, and mixtures of two or more thereof, wherein more preferably the one or more oxidic binders comprise one or more sources of silica, wherein more preferably the one or more binders consist of one or more sources of silica.
13. The catalyst molding of embodiment 12, comprising the one or more oxidic binders, calculated as the oxide, in an amount in the range of from 5 to 40 weight-%, more preferably of from 10 to 30 weight-%, more preferably of from 15 to 25 weight-%, more preferably of from 16 to 20 weight-%, based on the weight of the catalyst molding.
14. The catalyst molding of embodiment 12 or 13, wherein from 95 to 100 weight-%, more preferably from 98 to 100 weight-%, more preferably from 99 to 100 weight-%, more preferably from 99.5 to 100 weight-%, more preferably from 99.9 to 100 weight-% of the catalyst molding consist of the zeolitic material and the one or more oxidic binders.
15. The catalyst molding of any one of embodiments 1 to 14, exhibiting a water adsorption in the range of from 1.0 to 15.0 weight-%, preferably in the range of from 1.25 to 10.0 weight-%, more preferably in the range of from 1.5 to 8.0 weight-%, more preferably in the range of from 2.5 to 7.0 weight-%, more preferably in the range of from 3.5 to 6.5 weight-%, more preferably in the range of from 4.0 to 6.2 weight-%, more preferably in the range of from 4.3 to 6.0 weight-%, more preferably in the range of from 4.5 to 5.8 weight-%, wherein the water adsorption is preferably determined according to Reference Example 1.1.
16. The catalyst molding of embodiments 15, wherein the catalyst molding exhibits a water adsorption in the range of from 3 to 6.5 wt.-%, preferably of from 3.5 to 6 wt.-%, more preferably of from 4.4 to 5.5 wt.-%.
17. The catalyst molding of any one of embodiments 1 to 16, having a Ti content in the range of from 0.4 to 1.85 weight-%, preferably in the range of from 0.5 to 1.7 weight-%, more preferably in the range of from 0.6 to 1.5 weight-%, more preferably in the range of from 0.7 to 1.3 weight-%, more preferably in the range of from 0.8 to 1.2 weight-%, calculated as elemental Ti and based on the sum of the weights of the zeolitic material and optionally the one or more oxidic binders.
18. The catalyst molding of any one of embodiments 1 to 17, having a Si content in the range of from 36 to 48 weight-%, preferably in the range of from 38 to 46 weight-%, more preferably in the range of from 39 to 45 weight-%, calculated as elemental Si and based on the sum of the weights of the zeolitic material and optionally the one or more oxidic binders.
19. The catalyst molding of any one of embodiments 1 to 18, having a crystallinity in the range of from 40 to 90 weight.-%, preferably in the range of from 45 to 100 weight-%, more preferably in the range of from 50 to 90 weight-%, more preferably in the range of from 50 to 80 weight-%, wherein the crystallinity is preferably determined according to Reference Example 1.3.
20. The catalyst molding of any one of embodiments 1 to 19, displaying a water adsorption (W), preferably determined according to Reference Example 1.1, a concentration (C) of bridging μ² η²-peroxo species per Ti in the H₂O₂-activated catalyst molding, as determined by quantitative ¹⁷O NMR spectroscopy, preferably determined according to Reference Example 1.2, and an activation factor (A) according to formula I, wherein the activation factor is in the range of from 10 to 75 mmol/mol;
   wherein in accordance with formula I, the activation factor is the multiplication product of the water adsorption and the concentration of bridging μ² η²-peroxo species per Ti in the H₂O₂-activated catalyst molding:

A=W×C (I).
21. The catalyst molding of embodiment 20, displaying a concentration of bridging μ² η²-peroxo species per Ti in the H₂O₂-activated catalyst molding in the range of from 100 to 1,000 mmol/mol, preferably from 200 to 900 mmol/mol, more preferably from 300 to 800 mmol/mol, more preferably from 400 to 700 mmol/mol, more preferably from 420 to 680 mmol/mol, more preferably from 480 to 620 mmol/mol, and more preferably from 500 to 600 mmol/mol.
22. The catalyst molding of embodiment 20 or 21, wherein the concentration (C) of bridging μ² η²-peroxo species per Ti in the H₂O₂-activated catalyst molding as determined by quantitative ¹⁷O NMR spectroscopy is the concentration which is determined at a time point T after having brought the catalyst molding into contact with H₂¹⁷O₂,
   wherein T is in the range of from 1 to 720 min after having brought the catalyst molding into contact with H₂¹⁷O₂, preferably from 2 min to 480 min after having brought the catalyst molding into contact with H₂¹⁷O₂, more preferably from 4 to 240 min after having brought the catalyst molding into contact with H₂¹⁷O₂, more preferably from 6 to 120 min after having brought the catalyst molding into contact with H₂¹⁷O₂, more preferably from 8 to 60 min after having brought the catalyst molding into contact with H₂¹⁷O₂, more preferably from 10 to 30 min after having brought the catalyst molding into contact with H₂¹⁷O₂, more preferably from 12 to 20 min after having brought the catalyst molding into contact with H₂¹⁷O₂, and more preferably from 14 to 16 min after having brought the catalyst molding into contact with H₂¹⁷O₂, wherein more preferably T is 15 min after having brought the catalyst molding into contact with H₂¹⁷O₂.
23. The catalyst molding of any one of embodiments 20 to 22, wherein the activation factor of the catalyst molding is in the range of from 12 to 70 mmol/mol, preferably from 14 to 65 mmol/mol, more preferably from 16 to 60 mmol/mol, more preferably from 18 to 55 mmol/mol, more preferably from 19 to 50 mmol/mol, more preferably from 20 to 48 mmol/mol, more preferably from 23 to 46 mmol/mol, more preferably from 26 to 42 mmol/mol, more preferably from 28 to 39 mmol/mol, and more preferably from 30 to 36 mmol/mol.
24. The catalyst molding of embodiment 23, displaying a concentration of bridging μ² η²-peroxo species per Ti in the H₂O₂-activated catalyst molding in the range of from 100 to 1,000 mmol/mol, wherein the concentration of bridging μ² η²-peroxo species per Ti in the H₂O₂-activated catalyst molding as determined by quantitative ¹⁷O NMR spectroscopy is the concentration which is determined at a time point T after having brought the catalyst molding into contact with H₂¹⁷O₂, wherein T is in the range of from 65 to 175 min.
25. The catalyst molding of embodiment 24, displaying a concentration of bridging μ² η2-peroxo species per Ti in the H₂O₂-activated catalyst molding as determined by quantitative ¹⁷O NMR spectroscopy, preferably determined according to Reference Example 1.2, in the range of from 200 to 900 mmol/mol, preferably from 300 to 800 mmol/mol, more preferably from 380 to 750 mmol/mol, more preferably from 420 to 700 mmol/mol, more preferably from 470 to 650 mmol/mol, and more preferably from 500 to 620 mmol/mol.
26. The catalyst molding of embodiment 24 or 25, wherein T is in the range of from 75 to 165 min after having brought the catalyst molding into contact with H₂¹⁷O₂, preferably from 85 min to 155 min after having brought the catalyst molding into contact with H₂¹⁷O₂, more preferably from 95 to 145 min after having brought the catalyst molding into contact with H₂¹⁷O₂, more preferably from 105 to 135 min after having brought the catalyst molding into contact with H₂¹⁷O₂, more preferably from 112 to 128 min after having brought the catalyst molding into contact with H₂¹⁷O₂, more preferably from 116 to 124 min after having brought the catalyst molding into contact with H₂¹⁷O₂, more preferably from 118 to 122 min after having brought the catalyst molding into contact with H₂¹⁷O₂, and more preferably from 119 to 121 min after having brought the catalyst molding into contact with H₂¹⁷O₂, wherein more preferably T is 120 min after having brought the catalyst molding into contact with H₂¹⁷O₂.
27. The catalyst molding of any one of embodiments 24 to 26, wherein the activation factor of the zeolitic material is in the range of from 12 to 70 mmol/mol, preferably from 14 to 65 mmol/mol, more preferably from 16 to 60 mmol/mol, more preferably from 18 to 55 mmol/mol, more preferably from 19 to 50 mmol/mol, more preferably from 20 to 45 mmol/mol, more preferably from 21 to 40 mmol/mol, more preferably from 22 to 37 mmol/mol, more preferably from 23 to 35 mmol/mol, and more preferably from 24 to 34 mmol/mol.
28. The catalyst molding of any one of embodiments 1 to 27, having a BET specific surface area in the range of from 200 to 450 m²/g, preferably in the range of from 220 to 420 m²/g, more preferably in the range of from 240 to 400 m²/g, more preferably in the range of from 250 to 390 m²/g, wherein the BET specific surface area is preferably determined according to Reference Example 1.4.
29. The catalyst molding of any one of embodiments 1 to 28, wherein the UV-vis spectrum of the catalyst molding displays a first absorption band A1 having a maximum in the range of from 200 to 240 nm, wherein the UV-vis spectrum of the catalyst molding preferably displays a second absorption band A2 having a maximum in the range of from 241 to 330 nm, preferably of from 241 to 320 nm, and more preferably of from 241 to 300 nm.
30. The catalyst molding of any one of embodiments 1 to 29, showing a selectivity towards the sum of 1-methoxy-2-propanol and 2-methoxy-1-propanol in the range of from 0 to 15%, preferably in the range of from 0.1 to 9%, more preferably in the range of from 0.1 to 6.5%, preferably determined according to Example 14, more preferably determined according to Example 14 after a runtime in the range of from 22 to 24 h, more preferably determined according to Example 14 after a runtime of 24 h.
31. The catalyst molding of any one of embodiments 1 to 30, showing a selectivity towards the sum of 1-methoxy-2-propanol and 2-methyoxy-1-propanol in the range of from 0 to 15%, preferably in the range of from 0.1 to 9%, more preferably in the range of from 0.1 to 6.5%, preferably determined according to Example 14, more preferably determined according to Example 14 when the feed stream reaches full load of hydrogen peroxide for the first time.
32. The catalyst molding of any one of embodiments 1 to 31, showing a deactivation rate in the range of from 0 to 0.055 K/h, preferably in the range of from 0.001 to 0.035 K/h, wherein the deactivation rate is determined as described in Example 14.
33. The catalyst molding of any one of embodiments 1 to 32, wherein the catalyst molding is an extrudate or a granule.
34. The catalyst molding of any one of embodiments 1 to 33, being in the form of a strand or a sphere.
35. The catalyst molding of any one of embodiments 1 to 34, wherein the zeolitic material comprised in the catalyst molding exhibits a water adsorption in the range of from 1 to 6.9 weight-%, preferably in the range of from 1.25 to 6.8 weight-%, more preferably in the range of from 1.5 to 6.5 weight-%, more preferably in the range of from 1.5 to 6.25 weight-%, more preferably in the range of from 2.0 to 6.0 weight-%, more preferably in the range of from 2.5 to 5.75 weight-%, more preferably in the range of from 3.0 to 5.5 weight-%, more preferably in the range of from 3.25 to 5.25 weight-%, more preferably in the range of from 3.5 to 5.0 weight-%, more preferably in the range of from 3.75 to 4.75 weight-%, more preferably in the range of from 4.0 to 4.5 weight-%, wherein the water adsorption is preferably determined according to Reference Example 1.1.
36. The catalyst molding of embodiment 35, wherein the zeolitic material comprised in the catalyst molding exhibits a water adsorption in the range of from 3 to 6.5 wt.-%, preferably from 3.2 to 4.5 wt.-%, more preferably from 3.4 to 4.1 wt.-%.
37. The catalyst molding of any one of embodiments 1 to 36, wherein from 98 to 100 weight-%, preferably from 99 to 100 weight-%, more preferably from 99.5 to 100 weight-%, more preferably from 99.9 to 100 weight-%, of the zeolitic material comprised in the catalyst molding and having framework type MFI consist of Ti, Si, O, and H.
38. The catalyst molding of any one of embodiments 1 to 37, wherein the zeolitic material comprised in the catalyst molding has a Ti content in the range of from 0.3 to 3.0 weight-%, preferably in the range of from 0.4 to 2.0 weight-%, more preferably in the range of from 0.5 to 1.5 weight-%, more preferably in the range of from 0.6 to 1.4 weight-%, more preferably in the range of from 0.7 to 1.3 weight-%, more preferably in the range of from 0.8 to 1.2 weight-%, calculated as elemental Ti and based on the weight of the zeolitic material.
39. The catalyst molding of embodiment 38, wherein the zeolitic material comprised in the catalyst molding has a Ti content in the range of from 0.7 to 2.5 weight-%, preferably in the range of from 1.0 to 1.9 weight-%, more preferably in the range of from 1.1 to 1.3 weight-%, calculated as elemental Ti and based on the weight of the zeolitic material.
40. The catalyst molding of any one of embodiments 1 to 39, wherein the zeolitic material comprised in the catalyst molding has a Si content in the range of from 36 to 48 weight-%, preferably in the range of from 38 to 46 weight-%, more preferably in the range of from 39 to 45 weight-%, calculated as elemental Si and based on the weight of the zeolitic material.
41. The catalyst molding of any one of embodiments 1 to 40, wherein the zeolitic material comprised in the catalyst molding displays a water adsorption (W), preferably determined according to Reference Example 1.1, a concentration (C) of bridging μ² η²-peroxo species per Ti in the H₂O₂-activated zeolitic material, as determined by quantitative ¹⁷O NMR spectroscopy, preferably determined according to Reference Example 1.2, and an activation factor (A) according to formula I, wherein the activation factor is in the range of from 10 to 75 mmol/mol;
   wherein in accordance with formula I, the activation factor is the multiplication product of the water adsorption and the concentration of bridging μ²η²-peroxo species per Ti in the H₂O₂-activated zeolitic material:

A=W×C (I).
42. The catalyst molding of embodiment 41, wherein the zeolitic material comprised in the catalyst molding displays a concentration of bridging μ² η²-peroxo species per Ti in the H₂O₂-activated zeolitic material in the range of from 100 to 1,000 mmol/mol, preferably from 200 to 900 mmol/mol, more preferably from 300 to 850 mmol/mol, more preferably from 400 to 800 mmol/mol, more preferably from 500 to 750 mmol/mol, more preferably from 600 to 700 mmol/mol, and more preferably from 640 to 680 mmol/mol.
43. The catalyst molding of embodiment 41 or 42, wherein the concentration (C) of bridging μ² η²-peroxo species per Ti in the H₂O₂-activated zeolitic material as determined by quantitative ¹⁷O NMR spectroscopy is the concentration which is determined at a time point T after having brought the zeolitic material into contact with H₂¹⁷O₂, wherein T is in the range of from 1 to 720 min after having brought the zeolitic material into contact with H₂¹⁷O₂, preferably from 2 min to 480 min after having brought the zeolitic material into contact with H₂¹⁷O₂, more preferably from 4 to 240 min after having brought the zeolitic material into contact with H₂¹⁷O₂, more preferably from 6 to 120 min after having brought the zeolitic material into contact with H₂¹⁷O₂, more preferably from 8 to 60 min after having brought the zeolitic material into contact with H₂¹⁷O₂, more preferably from 10 to 30 min after having brought the zeolitic material into contact with H₂¹⁷O₂, more preferably from 12 to 20 min after having brought the zeolitic material into contact with H₂¹⁷O₂, and more preferably from 14 to 16 min after having brought the zeolitic material into contact with H₂¹⁷O₂, wherein more preferably T is 15 min after having brought the zeolitic material into contact with H₂¹⁷O₂.
44. The catalyst molding of any one of embodiments 41 to 43, wherein the activation factor of the zeolitic material comprised in the catalyst molding is in the range of from 12 to 70 mmol/mol, preferably from 14 to 65 mmol/mol, more preferably from 16 to 60 mmol/mol, more preferably from 18 to 55 mmol/mol, more preferably from 20 to 50 mmol/mol, more preferably from 22 to 48 mmol/mol, more preferably from 23 to 43 mmol/mol, more preferably from 24 to 39 mmol/mol, more preferably from 25 to 35 mmol/mol, and more preferably from 26 to 32 mmol/mol.
45. The catalyst molding of embodiment 41, wherein the zeolitic material comprised in the catalyst molding displays a concentration of bridging μ² η²-peroxo species per Ti in the H₂O₂-activated zeolitic material in the range of from 100 to 1,000 mmol/mol, wherein the concentration of bridging μ² η²-peroxo species per Ti in the H₂O₂-activated zeolitic material as determined by quantitative ¹⁷O NMR spectroscopy is the concentration which is determined at a time point T after having brought the zeolitic material into contact with H₂¹⁷O₂, wherein T is in the range of from 65 to 175 min.
46. The catalyst molding of embodiment 45, wherein the zeolitic material comprised in the catalyst molding displays a concentration of bridging μ² η²-peroxo species per Ti in the H₂O₂-activated zeolitic material as determined by quantitative ¹⁷O NMR spectroscopy, preferably determined according to Reference Example 1.2, in the range of from 200 to 900 mmol/mol, preferably from 300 to 800 mmol/mol, more preferably from 400 to 750 mmol/mol, more preferably from 460 to 700 mmol/mol, more preferably from 510 to 650 mmol/mol, and more preferably from 550 to 610 mmol/mol.
47. The catalyst molding of embodiment 45 or 46, wherein T is in the range of from 75 to 165 min after having brought the zeolitic material into contact with H₂¹⁷O₂, preferably from 85 min to 155 min after having brought the zeolitic material into contact with H₂¹⁷O₂, more preferably from 95 to 145 min after having brought the zeolitic material into contact with H₂¹⁷O₂, more preferably from 105 to 135 min after having brought the zeolitic material into contact with H₂¹⁷O₂, more preferably from 112 to 128 min after having brought the zeolitic material into contact with H₂¹⁷O₂, more preferably from 116 to 124 min after having brought the zeolitic material into contact with H₂¹⁷O₂, more preferably from 118 to 122 min after having brought the zeolitic material into contact with H₂¹⁷O₂, and more preferably from 119 to 121 min after having brought the zeolitic material into contact with H₂¹⁷O₂, wherein more preferably T is 120 min after having brought the zeolitic material into contact with H₂¹⁷O₂.
48. The catalyst molding of any one of embodiments 45 to 47, wherein the activation factor of the zeolitic material comprised in the catalyst molding is in the range of from 12 to 70 mmol/mol, preferably from 14 to 65 mmol/mol, more preferably from 16 to 60 mmol/mol, more preferably from 18 to 55 mmol/mol, more preferably from 19 to 50 mmol/mol, more preferably from 20 to 45 mmol/mol, more preferably from 21 to 40 mmol/mol, more preferably from 22 to 35 mmol/mol, more preferably from 23 to 31 mmol/mol, and more preferably from 24 to 28 mmol/mol.
49. The catalyst molding of any one of embodiments 1 to 48, wherein the zeolitic material comprised in the catalyst molding has a molar ratio of Ti atoms comprised in the framework structure of the zeolitic material to the total amount of Ti atoms comprised in the zeolitic material in the range of from 0.5:1 to 1:1, preferably in the range of from 0.55:1 to 1:1, more preferably in the range of from 0.6:1 to 1:1, preferably determined via X-ray photoelectron spectroscopy (XPS).
50. The catalyst molding of any one of embodiments 1 to 49, wherein the zeolitic material comprised in the catalyst molding has a Na content, calculated as Na₂O, in the range of from 0 to 0.5 weight-%, preferably of from 0 to 0.2 weight-%, more preferably of from 0 to 0.15 weight-%, more preferably of from 0 to 0.14 weight-%, more preferably of from 0 to 0.1 weight-%, more preferably in the range of from 0 to 0.07 weight-%, more preferably in the range of from 0 to 0.05 weight-%, based on the weight of the zeolitic material.
51. The catalyst molding of any one of embodiments 1 to 50, wherein the zeolitic material comprised in the catalyst molding has a content of one or more of Fe, Co, Ni, and Cu, calculated as Fe₂O₃, C₂O₃, NiO, and CuO, respectively, in the range of from 0 to 0.1 weight-%, preferably in the range of from 0 to 0.07 weight-%, more preferably in the range of from 0 to 0.05 weight-%, more preferably in the range of from 0 to 0.02 weight-%, based on the weight of the zeolitic material.
52. The catalyst molding of any one of embodiments 1 to 50, wherein the zeolitic material comprised in the catalyst molding has a content of one or more of Fe, Co, Ni, and Cu, calculated as the element, respectively, in the range of from 0 to 0.1 weight-%, preferably in the range of from 0 to 0.07 weight-%, more preferably in the range of from 0 to 0.05 weight-%, more preferably in the range of from 0 to 0.02 weight-%, based on the weight of the zeolitic material
53. The catalyst molding of any one of embodiments 1 to 52, wherein the zeolitic material comprised in the catalyst molding has an B content, calculated as B₂O₃, in the range of from 0 to 0.1 weight-%, preferably in the range of from 0 to 0.01 weight-%, more preferably in the range of from 0 to 0.001 weight-%, based on the weight of the zeolitic material.
54. The catalyst molding of any one of embodiments 1 to 53, wherein the zeolitic material comprised in the catalyst molding has a Ge content, calculated as GeO₂, in the range of from 0 to 0.1 weight-%, preferably in the range of from 0 to 0.01 weight-%, more preferably in the range of from 0 to 0.001 weight-%, based on the weight of the zeolitic material.
55. The catalyst molding of any one of embodiments 1 to 54, wherein the zeolitic material comprised in the catalyst molding has a C content, calculated as elemental C, in the range of from 0 to 0.1 weight-%, preferably in the range of from 0 to 0.01 weight-%, more preferably in the range of from 0 to 0.001 weight-%, based on the weight of the zeolitic material.
56. The catalyst molding of any one of embodiments 1 to 55, wherein the zeolitic material comprised in the catalyst molding has a crystallinity in the range of from 50 to 110 weight, preferably in the range of from 50 to 100 weight-%, more preferably in the range of from 70 to 100 weight-%, more preferably in the range of from 80 to 100 weight-%, wherein the crystallinity is preferably determined according to Reference Example 1.3.
57. The catalyst molding of any one of embodiments 1 to 56, wherein the zeolitic material comprised in the catalyst molding displays a BET specific surface area in the range of from 370 to 520 m²/g, preferably in the range of from 390 to 500 m²/g, more preferably in the range of from 410 to 480 m²/g, more preferably in the range of from 430 to 460 m²/g, wherein the BET specific surface area is preferably determined according to Reference Example 1.4.
58. The catalyst molding of embodiment 57, wherein the zeolitic material comprised in the catalyst molding displays a BET specific surface area in the range of from 390 to 440 m²/g.
59. The catalyst molding of any one of embodiments 1 to 58, wherein the zeolitic material comprised in the catalyst molding exhibits a type IV nitrogen adsorption/desorption isotherm, wherein the nitrogen adsorption/desorption isotherm is preferably determined according to Reference Example 1.8.
60. The catalyst molding of any one of embodiments 1 to 59, wherein the zeolitic material comprised in the catalyst molding is a TS-1 zeolite.
61. The catalyst molding of any one of embodiments 1 to 60, wherein the zeolitic material comprised in the catalyst molding exhibits a propylene oxide activity of at least 2.0 weight-%, preferably in the range of from 3.0 to 15.0 weight-%, more preferably in the range of from 5.0 to 14.0 weight-%, more preferably in the range of from 9.0 to 13.0 weight-%, preferably determined as described in Reference Example 1.5.
62. The catalyst molding of any one of embodiment 1 to 61, wherein the catalyst molding has a bulk density of equal to or greater than 320 g/l, preferably equal to or greater than 330 g/l, more preferably equal to or greater than 350 g/l, more preferably in the range of from 350 to 600 g/l, more preferably in the range of from 350 to 500 g/l, more preferably in the range of from 360 to 400 g/l, more preferably in the range of from 370 to 400 g/l, more preferably in the range of from 370 to 390 g/l, wherein the bulk density is preferably determined according to Reference Example 1.11.
63. The catalyst molding of any one of embodiment 1 to 62, wherein the catalyst molding exhibits a propylene oxide activity of at least 2.0 weight-%, preferably in the range of from 3.0 to 15.0 weight-%, more preferably in the range of from 3.5 to 10.0 weight-%, more preferably in the range of from 4.0 to 7.5 weight-%, more preferably in the range of from 4.5 to 6.5 weight-%, more preferably in the range of from 5.5 to 6.3 weight-%, preferably determined as described in Reference Example 1.5.
64. The catalyst molding of any one of embodiment 1 to 63, wherein the catalyst molding exhibits a k-80 test value of less than 0.20 h⁻¹, wherein preferably the catalyst molding exhibits a k-80 test value in the range of from 0.01 to 0.19 h⁻¹, more preferably of from 0.02 to 0.15 h⁻¹, more preferably of from 0.03 to 0.10 h⁻¹, more preferably of from 0.04 to 0.06 h⁻¹, preferably determined as described in Reference Example 1.6.
65. A reactor comprising a plurality of catalyst moldings, each of the catalyst moldings independently from one another being in accordance with the catalyst molding according to any one of embodiments 1 to 64, wherein from 42 to 100% of the plurality of catalyst moldings has an aspect ratio in the range of from greater than 1.5 to smaller than 6.1, preferably in the range of from greater than 1.5 to 6.0, more preferably in the range of from 1.8 to smaller than 6.1, and more preferably within the range of from 1.8 to 6.0, wherein the aspect ratio is preferably determined according to Reference Example 1.12.
66. The reactor of embodiment 65, wherein from 15 to 25%, preferably from 18 to 22%, of the plurality of catalyst moldings has an aspect ratio in the range of from 1.4 to smaller than 1.8.
67. The reactor of embodiment 65 or 66, wherein from 37 to 47%, preferably from 40 to 44%, of the plurality of catalyst moldings has an aspect ratio in the range of from 1.8 to smaller than 2.2.
68. The reactor of any one of embodiments 65 to 67, wherein from 19 to 29%, preferably from 22 to 26%, of the plurality of catalyst moldings has an aspect ratio in the range of from 2.2 to smaller than 2.7.
69. The reactor of any one of embodiments 65 to 68, wherein from 5 to 15%, preferably from 8 to 12%, of the plurality of catalyst moldings has an aspect ratio in the range of from 2.7 to smaller than 3.2.
70. The reactor of any one of embodiments 65 to 69, wherein from 1 to 11%, preferably from 4 to 8%, of the plurality of catalyst moldings has an aspect ratio in the range of from 3.6 to smaller than 4.1.
71. The reactor of any one of embodiments 65 to 69, wherein from 0 to 5%, preferably from 0 to 2%, of the plurality of catalyst moldings has an aspect ratio in the range of from 3.6 to smaller than 6.1, preferably in the range of from 3.6 to smaller than 4.6.
72. The reactor of embodiment 65, wherein from 1 to 22%, preferably from 2 to 20%, of the plurality of catalyst moldings has an aspect ratio in the range of from 1.0 to 1.5.
73. The reactor of embodiment 65 or 72, wherein from 10 to 46%, preferably from 12 to 44%, of the plurality of catalyst moldings has an aspect ratio in the range of from greater than 1.5 to 2.0.
74. The reactor of any one of embodiments 65, 72 and 73, wherein from 9 to 31%, preferably from 13 to 27%, more preferably from 15 to 25%, of the plurality of catalyst moldings has an aspect ratio in the range of from greater than 2.0 to 2.5.
75. The reactor of any one of embodiments 65 and 72 to 74, wherein from 7 to 27%, preferably from 9 to 25%, of the plurality of catalyst moldings has an aspect ratio in the range of from greater than 2.5 to 3.0.
76. The reactor of any one of embodiments 65 and 72 to 75, wherein from 1 to 24%, preferably from 3 to 22%, of the plurality of catalyst moldings has an aspect ratio in the range of from greater than 3.0 to 3.5.
77. The reactor of any one of embodiments 65 and 72 to 76, wherein from 0 to 17, preferably from 2 to 15%, of the plurality of catalyst moldings has an aspect ratio in the range of from greater than 3.5 to 4.0.
78. The reactor of any one of embodiments 65 and 72 to 77, wherein from 0 to 10, preferably from 1 to 8%, of the plurality of catalyst moldings has an aspect ratio in the range of from greater than 4.0 to 4.5.
79. The reactor of any one of embodiments 65 and 72 to 78, wherein from 0 to 16, preferably from 1 to 14%, of the plurality of catalyst moldings has an aspect ratio in the range of from greater than 4.5 to 5.0, preferably in the range of from greater than 4.5 to 6.0.
80. A process for preparing a catalyst molding according to any one of embodiments 1 to 64, the process comprising
   (i) preparing a mixture comprising one or more binder precursors and a zeolitic material having framework type MFI, preferably a zeolitic material as defined in any one of embodiments 35 to 61;
   (ii) shaping the mixture obtained from (i) to a catalyst molding precursor, obtaining a precursor of the catalyst molding;
   (iii) optionally preparing a mixture comprising the precursor of the catalyst molding obtained from (ii) and water, and subjecting the mixture to a water treatment under hydrothermal conditions, obtaining a water-treated precursor of the catalyst molding;
   (iv) calcining the precursor of the catalyst molding obtained from (ii) or the water-treated precursor of the catalyst molding obtained from (iii) in a gas atmosphere, wherein the gas atmosphere preferably comprises, more preferably consists of, one or more of nitrogen and oxygen, preferably air, obtaining the catalyst molding.
81. The process of embodiment 80, wherein the one or more binder precursors are selected from the group consisting of a silica sol, a colloidal silica, a wet process silica, a dry process silica, and a mixture of two or more thereof, wherein the one or more binder precursors more preferably comprise, preferably consist of, a colloidal silica.
82. The process of embodiment 80 or 81, wherein the one or more binder precursors comprise Si, wherein in the mixture according to (i), the weight ratio of zeolitic material, relative to Si comprised in the one or more binder precursors, calculated as SiO₂, is preferably in the range of from 1.5:1 to 10:1, more preferably of from 1.8:1 to 6:1, more preferably of from 2:1 to 5.6:1, more preferably of from 2.3:1 to 4:1, more preferably of from 2.5:1 to 3.5:1, more preferably of from 2.9:1 to 3.1:1.
83. The process of embodiment 82, wherein the weight ratio of zeolitic material, relative to Si comprised in the one or more binder precursors, calculated as SiO₂, is in the range of from 2:1 to 10:1, preferably in the range of from 5:1 to 7.5:1, more preferably in the range of from 6.0:1 to 6.5:1.
84. The process of embodiment 82, wherein the weight ratio of zeolitic material, relative to Si comprised in the one or more binder precursors, calculated as SiO₂, is in the range of from 2:1 to 10:1, preferably in the range of from 4:1 to 6:1, more preferably in the range of from 4.8:1 to 5.0:1.
85. The process of any one of embodiments 80 to 84, wherein the mixture prepared according to (i) further comprises one or more agents, wherein the agents comprise, preferably consist of, pore forming agents, preferably mesopore forming agents, lubricants, and plasticizers.
86. The process of embodiment 85, wherein the one or more agents comprise, preferably consist of, one or more of water, alcohols, organic polymers, carbohydrates, graphite, plant additives, and mixtures of two or more thereof, preferably one or more of water, polymeric vinyl compounds, polysaccharides, polyalkylene oxides, polystyrenes, polyacrylates, polymethacrylates, polyolefins, polyamides, polyesters, and mixtures of two or more thereof, more preferably one or more of water, polymeric vinyl compounds, celluloses, cellulose derivatives, polymannoses, polymannose derivatives, starches, Sesbania cannabina leaf powder, Sesbania cannabina gum powder, polyalkylene oxides, polystyrenes, polyacrylates, polymethacrylates, polyolefins, polyamides, polyesters, and mixtures of two or more thereof, more preferably one or more of water, polymeric vinyl compounds, polyalkylene oxides, polyacrylates, polyolefins, polystyrenes, polyamides, polyesters, celluloses, cellulose derivatives, polymannoses, polymannose derivatives, Sesbania cannabina leaf powder, Sesbania cannabina gum powder, and mixtures of two or more thereof, more preferably one or more of water, polystyrene, C₂ to C₃ polyalkylene oxides, polymeric vinyl compounds, cellulose, hydroxymethylcellulose, methoxy cellulose, ethoxy cellulose, polymannose at least partially derivatised with galactose, Sesbania cannabina leaf powder, Sesbania cannabina gum powder, and mixtures of two or more thereof, more preferably one or more of water, polystyrene, polyethylene oxide, a polyvinylacetate, a cellulose, a galactomannan, Sesbania cannabina leaf powder, Sesbania cannabina gum powder, and mixtures of two or more thereof, more preferably one or more of water, polyethylene oxide, a polyvinylacetate, a cellulose, a galactomannan, Sesbania cannabina leaf powder, Sesbania cannabina gum powder, and mixtures of two or more thereof, wherein the one or more agents more preferably comprise, more preferably consist of, one or more of water, a polyvinylacetate, a cellulose, a galactomannan, Sesbania cannabina leaf powder, Sesbania cannabina gum powder, and mixtures of two or more thereof.
87. The process of embodiment 85 or 86, wherein in the mixture prepared according to (i), the weight ratio of zeolitic material, relative to the one or more agents is in the range of from 1:1 to 99:1, more preferably in the range of from 1.3:1 to 95:1, more preferably in the range of from 1.5:1 to 90:1, more preferably in the range of from 1.8:1 to 70:1, more preferably in the range of from 2.0:1 to 50:1, more preferably in the range of from 2.3:1 to 30:1, more preferably in the range of from 2.5:1 to 15:1, more preferably in the range of from 2.8:1 to 8:1, more preferably in the range of from 3:1 to 4:1.
88. The process of embodiment 87, wherein the weight ratio of zeolitic material, relative to the one or more agents is in the range of from 1:1 to 5:1, preferably in the range of from 2.5:1 to 4:1, more preferably in the range of from 1.8:1 to 2.0:1.
89. The process of any one of embodiments 80 to 88, wherein the mixture prepared according to (i) comprises from 0 to 0.1 weight-%, preferably from 0 to 0.01 weight-%, more preferably from 0 to 0.001 weight-%, of ammonia, preferably of a hydrolyzing agent, based on the weight of the mixture.
90. The process of any one of embodiments 80 to 89, wherein the mixture prepared according to (i) is mixed in a kneader or in a mix-muller.
91. The process of any one of embodiments 80 to 90, wherein in (ii), the mixture is shaped to a catalyst molding precursor having an aspect ratio D1:D2 in the range of from 1.4:1 to 6.1:1, preferably in the range of from 1.45:1 to 5.6:1, more preferably in the range of from 1.5:1 to 5.1:1, more preferably in the range of from 1.55:1 to 4.6:1, more preferably in the range of from 1.6:1 to 4.1:1, more preferably in the range of from 1.65:1 to 3.6:1, more preferably in the range of from 1.7:1 to 3.2:1, more preferably in the range of from 1.75:1 to 2.7:1, more preferably in the range of from 1.8:1 to 2.2:1, wherein the aspect ratio is preferably determined according to Reference Example 1.12.
92. The process of any one of embodiments 80 to 91, wherein in (ii), the mixture is shaped to a catalyst molding precursor, wherein D1 is in the range of from 0.1 to 10 mm, more preferably in the range of from 1.2 to 7.0 mm, more preferably in the range of from 2.2 to 5.0 mm, more preferably in the range of from 3.0 to 4.0 mm, more preferably in the range of from 3.4 to 3.8 mm.
93. The process of any one of embodiments 80 to 92, wherein in (ii), the mixture is shaped to a catalyst molding precursor, wherein D2 is in the range of from 0.05 to 5 mm, preferably in the range of from 0.6 to 3.5 mm, more preferably in the range of from 1.1 to 2.5 mm, more preferably in the range of from 1.5 to 2.0 mm, more preferably in the range of from 1.7 to 1.9.
94. The process of any one of embodiments 80 to 93, wherein in (ii), the mixture is shaped to a catalyst molding precursor having a cross-sectional profile, wherein the cross-sectional profile is circular, hexagonal, rectangular, quadratic, triangular, oval, a starshaped polygon, or cloverleaf-shaped, preferably circular, hexagonal, rectangular, quadratic, triangular, oval, a star-shaped polygon having 3, 4, 5, 6, 7, or 8 tips, a trilobe or a quadrilobe, more preferably circular, hexagonal, rectangular, quadratic, triangular, oval, a star-shaped polygon having 3 or 4 tips, a trilobe or a quadrilobe.
95. The process of embodiment 94, wherein the cross-sectional profile is a star-shaped polygon having 3 tips, wherein the tips are rounded and wherein the corners are rounded.
96. The process of any one of embodiments 80 to 95, wherein in (ii), the mixture is shaped to a catalyst molding precursor, wherein P3 and P1 have a cutting angle in the range of from 60° to 90°, preferably in the range of from 70° to 90°, more preferably in the range of from 80° to 90°, more preferably in the range of from 85° to 90°, more preferably in the range of from 89° to 90°, wherein P3 more preferably is substantially perpendicular to P1.
97. The process of any one of embodiments 80 to 96, wherein in (ii), the mixture is shaped to a catalyst molding precursor having the shape of an extrudate, said shape having a length in the conceived or actual direction of extrusion, and said shape having a fixed cross-sectional profile perpendicular to said conceived or actual direction of extrusion.
98. The process of embodiment 97, wherein the catalyst molding having the shape of an extrudate has a circular cross-sectional profile with a diameter D, the diameter D corresponding to D2.
99. The process of any one of embodiments 80 to 98, wherein in (ii), shaping comprises extruding the mixture, optionally by piston press or extruder, preferably by screw extruder, more preferably by single or twin screw extruder, more preferably by single screw extruder.
100. The process of any one of embodiments 80 to 99, wherein shaping according to (ii) further comprises drying the precursor of the molding in a gas atmosphere, wherein the gas atmosphere preferably comprises, more preferably consists of, one or more of an inert gas, preferably nitrogen, and oxygen.
101. The process of embodiment 100, wherein drying is carried out at a temperature of the gas atmosphere in the range of from 80 to 160 °C, preferably in the range of from 100 to 140 °C, more preferably in the range of from 110 to 130 °C.
102. The process of any one of embodiments 80 to 101, wherein shaping according to (ii) further comprises, preferably after drying the precursor of the molding according to embodiment 100 or 101, calcining the precursor of the molding in a gas atmosphere,
wherein the gas atmosphere preferably comprises, more preferably consists of, one or more of an inert gas, preferably nitrogen, and oxygen.
103. The process of embodiment 102, wherein calcining is carried out at a temperature of the gas atmosphere in the range of from 350 to 700 °C, preferably in the range of from 400 to 490 °C, preferably in the range of from 420 to 470 °C, more preferably in the range of from 440 to 460 °C.
104. The process of any one of embodiments 80 to 103, wherein the water treatment according to (iii) treatment is performed with a water containing solvent system and/or with an aqueous solution, wherein preferably the hydrothermal treatment is performed with deionized water.
105. The process of any one of embodiments 80 to 104, wherein the water treatment according to (iii) comprises a temperature of the mixture in the range of from 100 to 200 °C, preferably in the range of from 125 to 175 °C, more preferably in the range of from 130 to 160 °C, more preferably in the range of from 135 to 155 °C more preferably in the range of from 140 to 150 °C.
106. The process of any one of embodiments 80 to 105, wherein the water treatment according to (iii) is carried out under autogenous pressure, preferably in an autoclave.
107. The process of any one of embodiments 80 to 106, wherein the water treatment according to (iii) is carried out for 6 to 15 h, preferably for 6 to 10 h, more preferably for 7 to 9 h, more preferably for 7.5 to 8.5 h.
108. The process of any one of embodiments 80 to 107, wherein in the mixture prepared in (iii), the weight ratio of the precursor of the catalyst molding obtained from (ii) relative to the water is in the range of from 1:5 to 1:25, preferably in the range of from 1:10 to 1:20, more preferably in the range of from 1:13 to 1:17.
109. The process of any one of embodiments 80 to 108, wherein after (iii) and prior to (iv), the water-treated precursor of the molding is separated from the mixture obtained from (iii), wherein separating preferably comprises subjecting the mixture obtained from (iii) to filtration or centrifugation, wherein more preferably, separating further comprises washing the water-treated precursor of the molding at least once with a liquid solvent system, wherein the liquid solvent system preferably comprises one or more of water, an alcohol, and a mixture of two or more thereof, wherein the water-treated precursor of the molding is more preferably washed with water.
110. The process of any one of embodiments 80 to 109, wherein after (iii) and prior to (iv), the preferably separated water-treated precursor of the molding is dried in a gas atmosphere,
wherein drying is preferably carried out at a temperature of the gas atmosphere in the range of from 80 to 160 °C, more preferably in the range of from 100 to 140 °C, more preferably in the range of from 110 to 130 °C, wherein the gas atmosphere preferably comprises air.
111. The process of any one of embodiments 80 to 110, wherein calcining according to (iv) is carried out at a temperature of the gas atmosphere in the range of from 350 to 700 °C, preferably in the range of from 400 to 490 °C, preferably in the range of from 420 to 470 °C, more preferably in the range of from 440 to 460 °C.
112. The process of any one of embodiments 80 to 111, wherein the zeolitic material having framework type MFI in the mixture prepared according to (i) is prepared according to a process comprising
   (a) providing a zeolitic material having framework type MFI;
   (b) preparing an aqueous mixture comprising the zeolitic material provided in (i) and a structure directing agent, wherein the structure directing agent preferably comprises, more preferably consists of, tetrapropylammonium hydroxide and/or tetrapropylammonium bromide, preferably tetrapropylammonium hydroxide;
   (c) subjecting the mixture obtained from (b) to hydrothermal conditions under autogenous pressure, preferably in an autoclave, obtaining a suspension comprising a precursor of the zeolitic material having framework type MFI as defined in any one of embodiments 35 to 61, and separating said precursor from the suspension;
   (d) optionally calcining the precursor obtained from (c) in a gas atmosphere, wherein the gas atmosphere preferably comprises, more preferably consists of, air;
   (e) optionally subjecting the precursor obtained from (c) or (d) to an acid treatment;
   (f) optionally drying the acid-treated precursor obtained from (e) in a gas atmosphere, wherein the gas atmosphere preferably comprises, more preferably consists of, air;
   (g) calcining the precursor obtained from (c), (e), or (f) in a gas atmosphere, wherein the gas atmosphere preferably comprises, more preferably consists of, air, obtaining the zeolitic material having framework type MFI according to any one of embodiments 35 to 61.
113. The process of embodiment 112, wherein from 98 to 100 weight-%, preferably from 99 to 100 weight-%, more preferably from 99.5 to 100 weight-%, more preferably from 99.9 to 100 weight-%, of the zeolitic material having framework type MFI provided in (a) consist of Ti, Si, O, and H.
114. The process of embodiment 112 or 113, wherein the zeolitic material provided in (a) exhibits a type I nitrogen adsorption/desorption isotherm, wherein the nitrogen adsorption/desorption isotherm is preferably determined according to Reference Example 1.8.
115. The process of any one of embodiments 112 to 114, wherein the zeolitic material provided in (a) has a Ti content in the range of from 0.3 to 3.0 weight-%, preferably in the range of from 0.4 to 2.0 weight-%, more preferably in the range of from 0.5 to 1.5 weight-%, more preferably in the range of from 0.6 to 1.4 weight-%, more preferably in the range of from 0.7 to 1.3 weight-%, more preferably in the range of from 0.8 to 1.2 weight-%, calculated as elemental Ti and based on the weight of the zeolitic material provided in (a).
116. The process of embodiment 115, wherein the zeolitic material provided in (a) has a Ti content in the range of from 0.7 to 2.5 weight-%, preferably in the range of from 1.0 to 1.9 weight-%, more preferably in the range of from 1.1 to 1.3 weight-%, calculated as elemental Ti and based on the weight of the zeolitic material provided in (a).
117. The process of any one of embodiments 112 to 116, wherein the aqueous mixture prepared in (b) comprises the zeolitic material provided in (a) and the structure directing agent in a weight ratio in the range of from 0.25:1 to 5:1, preferably in the range of from 0.75:1 to 1.5:1.
118. The process of any one of embodiments 112 to 117, wherein the aqueous mixture prepared in (b) comprises the zeolitic material provided in (a) and water in a weight ratio in the range of from 0.01:1 to 0.50:1, preferably in the range of from 0.10:1 to 0.20:1.
119. The process of any one of embodiments 112 to 118, wherein the aqueous mixture prepared in (b) further comprises NH₃ and/or a source of NH₃, wherein preferably the source of NH₃ comprises, preferably consists of, urea.
120. The process of any one of embodiments 112 to 119, wherein subjecting the aqueous mixture obtained from (b) according to (c) to hydrothermal conditions comprises heating the mixture to a temperature in the range of from 140 to 200 °C, preferably in the range of from 160 to 180 °C.
121. The process of any one of embodiments 112 to 120, wherein the aqueous mixture obtained from (b) is subjected according to (c) to hydrothermal conditions for a duration in the range of from 60 to 110 h, preferably in the range of from 75 to 95 h.
122. The process of any one of embodiments 112 to 121, wherein the precursor obtained from (c) is calcined according to (d) at a temperature in the range of from 450 to 530 °C, preferably in the range of from 470 to 510 °C.
123. The process of any one of embodiments 112 to 122, wherein the precursor obtained from (c) is calcined according to (d) for a duration in the range of from 2 to 8 h, preferably in the range of from 4 to 6 h.
124. The process of any one of embodiments 112 to 123, wherein the acid treatment according to (e) comprises bringing the precursor obtained from (c) or (d) in contact with an aqueous mixture comprising an acid, wherein the acid preferably comprises nitric acid, wherein the aqueous mixture more preferably comprises from 5 to 15 weight-% of nitric acid, based on the total weight of aqueous mixture.
125. The process of any one of embodiments 112 to 124, wherein the acid-treated precursor obtained from (e) is dried in (f) at a temperature in the range of from 100 to 140 °C, preferably in the range of from 110 to 130 °C.
126. The process of any one of embodiments 112 to 125, wherein the precursor obtained from (c), (e), or (f) is calcined according to (g) at a temperature in the range of from 450 to 530 °C, preferably in the range of from 470 to 510 °C.
127. The process of any one of embodiments 112 to 126, wherein the precursor obtained from (c), (e), or (f) is calcined according to (g) atmosphere for a duration in the range of from 2 to 8 h, preferably in the range of from 4 to 6 h.
128. A catalyst molding obtainable or obtained according to the process of any one of embodiments 80 to 127.
129. A process for the activation of hydrogen peroxide comprising:
   (1) providing a reactor comprising a catalyst molding according to any of embodiments 1 to 64 and 128 or a reactor according to any one of embodiments 65 to 79;
   (2) contacting the catalyst molding provided in (1) or the plurality of catalyst moldings comprised in the reactor provided in (1) with hydrogen peroxide.
130. The process of embodiment 129, wherein contacting in (2) is conducted at a temperature in the range of from 10 to 100 °C, preferably from 20 to 80 °C, more preferably from 25 to 75 °C, more preferably from 30 to 65 °C.
131. The process of embodiment 126 or 130, wherein contacting in (2) is conducted at a pressure in the range of from 5 to 100 bar, preferably from 10 to 50 bar, more preferably from 14 to 32 bar, more preferably from 15 to 25 bar, wherein the pressure is defined as the pressure at the exit of the reactor.
132. The process of any one of embodiments 129 to 131, wherein in (2) hydrogen peroxide is comprised in a liquid feed stream which is fed into the reactor, wherein the liquid feed stream further comprises one or more unsaturated organic compounds, preferably one or more olefins, more preferably one or more C₂ to C₅ alkenes, more preferably one or more C₂ to C₄ alkenes, more preferably one or more C₂ or C₃ alkenes, more preferably propylene.
133. The process of embodiment 132, wherein the liquid feed stream further comprises a solvent system, wherein the solvent system comprises one or more solvents, wherein preferably the solvent system comprises one or more hydrophilic solvents, the hydrophilic solvents preferably being selected from the group consisting of polar solvents, more preferably from the group consisting of polar protic solvents, wherein more preferably the solvent system comprises one or more polar protic solvents selected from the group consisting of water, alcohols, and mixtures of two or more thereof, more preferably from the group consisting of water, C₁ to C₅ alcohols, and mixtures of two or more thereof, more preferably from the group consisting of water, C₁ to C₄ alcohols, and mixtures of two or more thereof, more preferably from the group consisting of water, C₁ to C₃ alcohols, and mixtures of two or more thereof, more preferably from the group consisting of water, methanol, ethanol, propanol, and mixtures of two or more thereof, more preferably from the group consisting of water, methanol, and mixtures thereof, wherein more preferably the solvent system comprises water, preferably water and methanol, wherein more preferably the solvent system consists of water and methanol.
134. The process of embodiment 132 or 133, wherein the liquid feed stream further comprises a potassium-containing compound, preferably a potassium salt, more preferably one or more of dipotassium hydrogen phosphate and a potassium salt of etidronic acid.
135. The process of any one of embodiments 132 to 134, wherein the liquid feed stream comprises hydrogen peroxide at a concentration in the range of from 1 to 75 weight-%, preferably from 3 to 50 weight-%, 5 to 30 weight-%, more preferably from 7 to 25 weight-%, more preferably from 8 to 20 weight-%, more preferably from 9 to 15 weight-%, more preferably from 10 to 12 weight-%, based on the total weight of the liquid feed stream.
136. The process of any one of embodiments 132 to 135, wherein the liquid feed stream fed into the reactor in (2) has a temperature in the range of from 0 to 60 °C, preferably from 25 to 50 °C.
137. The process of any one of embodiments 132 to 136, wherein the liquid feed stream fed into the reactor in (2) is at a pressure in the range of from 5 to 100 bar, preferably from 10 to 50 bar, more preferably from 15 to 25 bar.
138. The process of any one of embodiments 132 to 137, wherein the loading of the catalyst molding in the reactor in (1) is in the range of from 0.05 to 5 h⁻¹, preferably from 0.1 to 3 h⁻¹, more preferably from 0.2 to 1 h⁻¹, more preferably from 0.200 to 0.5 h⁻¹, more preferably from 0.210 to 0.25 h⁻¹, more preferably from 0.215 to 0.240 h⁻¹, more preferably from 0.220 to 0.235 h⁻¹, more preferably from 0.225 to 0.230 h⁻¹, wherein the loading of the catalyst molding is defined as the ratio of the mass flow rate in kg/h of hydrogen peroxide contained in the liquid feed stream divided by the amount in kg of the catalyst molding comprised in the reactor in (1).
139. The process of any one of embodiments 129 to 138, wherein the process further comprises
   (3) removing an effluent stream from the reactor, the effluent stream comprising an oxidized organic compound, and preferably comprising an epoxidized organic compound, more preferably an alkylene oxide, more preferably an alkylene oxide selected from C₂ to C₅ alkylene oxides, more preferably from C₂ to C₄ alkylene oxides, more preferably from C₂ or C₃ alkylene oxides, more preferably from C₃ alkylene oxides, wherein more preferably the effluent stream comprises propylene oxide.
140. Use of the catalyst molding according to any one of embodiments 1 to 64 and 128, as a catalyst and/or catalyst component, or a reactor according to any one of embodiments 65 to 79, in a reaction involving one or more of C—O bond formation, C—C bond formation and C—C bond conversion, and preferably as a catalyst and/or catalyst component in an isomerization reaction, in an ammoximation reaction, in an amination reaction, in a hydrocracking reaction, in an alkylation reaction, in an acylation reaction, in a reaction for the conversion of alkanes to olefins, or in a reaction for the conversion of one or more oxygenates to olefins and/or aromatics, in a reaction for the synthesis of hydrogen peroxide, in an aldol condensation reaction, in a reaction for the isomerization of epoxides, in a transesterification reaction, in a hydroxylation reaction, in a Baeyer-Villiger-type oxidation reaction, in a Dakin-type reaction, or in an epoxidation reaction, preferably as a catalyst and/or catalyst component in a hydroxylation reaction, in a Baeyer-Villiger-type oxidation reaction, in a Dakin-type reaction, or in a reaction for the epoxidation of olefins, more preferably in a reaction for the epoxidation of olefins, more preferably in a reaction for the epoxidation of C₂ to C₅ alkenes, more preferably in a reaction for the epoxidation of C₂ to C₄ alkenes, in a reaction for the epoxidation of C₂ or C₃ alkenes, more preferably for the epoxidation of C₃ alkenes, and more preferably as a catalyst or catalyst component for the conversion of propylene to propylene oxide.
141. A process for preparing an olefin oxide comprising
   (A) providing olefin, hydrogen peroxide, water and organic solvent and optionally an additive into an epoxidation zone comprising the catalyst molding according to any one of embodiments 1 to 64 and 128, as a catalyst and/or catalyst component, or a reactor according to any one of embodiments 65 to 79, obtaining a reaction mixture comprising olefin, hydrogen peroxide, water and organic solvent;
   (B) subjecting the reaction mixture obtained from (A) to epoxidation reaction conditions in the epoxidation zone, thereby obtaining a mixture comprising olefin oxide, water and organic solvent;
   (C) removing an effluent stream from the epoxidation zone, comprising olefin oxide, water and organic solvent.
142. The process of embodiment 141,
   wherein the olefin is a C₂-C₁₀ alkene, preferably a C₂-C₅ alkene, more preferably a C₂-C₄ alkene, more preferably ethylene or propylene, more preferably propylene;
   and/or, preferably and,
   wherein the organic solvent is an organic epoxidation solvent, wherein preferably the organic solvent is selected from the group consisting of C₁ to C₅ mono alcohol, C₁ to C₅ ether, C₁ to C₅ nitrile, and mixtures of two or more thereof, more preferably from the group consisting of tert-butanol, methanol, acetonitrile, methyl tert-butyl ether (MTBE), and mixtures of two or more thereof;
   and/or, preferably and,
   wherein the reaction mixture obtained in (A) preferably comprises the additive,
   wherein the additive is more preferably selected from the group consisting of potassium salt of an inorganic acid, potassium salt of an organic acid, ammonia, ammonium salt of an inorganic acid, ammonium salt of an organic acid and mixtures of two or more thereof; wherein the additive preferably comprises a potassium-containing compound, preferably a potassium salt, more preferably one or more of dipotassium hydrogen phosphate and dipotassium etidronate (K₂HEDP).

The present invention is further illustrated by the following examples, comparative examples and reference examples.

### EXPERIMENTAL SECTION

### Reference Example 1: Determination Methods

### Reference Example 1.1: Determination of Water Adsorption

Determination of the water adsorption properties of the examples of the experimental section was performed on a VTI SA instrument from TA Instruments following a step-isotherm program. The experiment consisted of a run, or a series of runs performed on a sample material that has been placed on the microbalance pan inside of the instrument. Before a measurement was started, residual moisture of a sample was removed by heating the sample to 120 °C (heating ramp of 5 °C/min) and holding it for 6 h under a N₂ flow. After the drying program, the temperature in the cell was decreased to 25 °C and kept isothermal during the measurements. The microbalance was calibrated, and the weight of the dried sample was balanced (maximum mass deviation 0.01 wt. %). Water uptake by the sample was measured as the increase in weight over that of the dry sample. First, an adsorption curve was measured by increasing the relative humidity (RH) to which the sample was exposed and measuring the water uptake by the sample at equilibrium. The RH was increased with a step of 10% from 5 to 85% and at each step the system controlled the RH and monitored the sample weight until reaching the equilibrium conditions and recording the weight uptake. The total adsorbed water amount by the sample was taken after the sample was exposed to 85% RH and expressed as weight percent relative to the weight of the dried sample. During the desorption measurement the RH was decreased from 85% to 5% with a step of 10% and the change in the weight of the sample (water uptake) was monitored and recorded.

### Reference Example 1.2: Determination of the Concentration of Bridging μ² η²-Peroxo Species in an H₂O₂-Activated Catalyst Via ¹⁷O NMR Spectroscopy

All NMR measurements were obtained on a Bruker Avance III 600-MHz NMR spectrometer (14.1 T) at low temperatures (100 K) using a 3.2-mm probe. The chemical shift was referenced against the signal of H₂O (at 100 K) to 0 ppm. Measurements were performed in a 3.2-mm sapphire rotor closed with a zirconia cap. Static WURST-CPMG (wideband, uniform-rate and smooth-truncation pulse with CPMG echo-train acquisition) experiments were performed to obtain the ¹⁷O NMR spectra. Details of the WURST pulse were as follows: length, 50 μs; 80 steps; sweep width, 0.5 MHz, sweeping from low to high frequency. SPINAL64 with 100 kHz radio frequency was used for 1H decoupling.

A TS-1 sample with ¹⁷O-labelled H₂O₂ was prepared by impregnating 25 mg of a TS-1 zeolite with one molar equivalent (with respect to Ti) of a 1.6 M aqueous solution of ¹⁷O-labelled H₂O₂. The samples were left to equilibrate for 15 min or 2 h before spectroscopic measurements.

For the determination of the concentration of bridging μ² η²-peroxo species in an H₂O₂-activated molding, a sample was prepared by impregnating 25 mg of a molding comprising a TS-1 zeolite with one molar equivalent (with respect to Ti) of a 1.1 M aqueous solution of ¹⁷O-labelled H₂O₂. The samples were left to equilibrate for 15 min or 2 h before spectroscopic measurements.

As indicated above, a sample was probed using solid-state ¹⁷O NMR spectroscopy at 100 K. This approach enabled the observation of reaction intermediates that originate from the activation of H₂¹⁷O₂, while at low temperature possible signal averaging due to dynamics was avoided and peroxo decomposition is prevented. The solid-state ¹⁷O NMR spectra of H₂¹⁷O₂ and H₂¹⁷O were also recorded, because these molecules were probably present in the catalyst sample.

The solid-state NMR spectra of the samples contacted with one equivalent (with respect to Ti) of H₂¹⁷O₂ solution for 2 h showed that H₂¹⁷O₂ reacted in all cases and that two new main signals of comparable intensity appeared.

To probe peroxo formation rates and stability a quantification protocol was established. First, a WURST QCMPG spectrum was measured (as described above) and subsequently the echo spectrum was reconstructed and then deconvolved into components associated with H₂O, H₂O₂ and peroxo species.

To avoid overfitting, the experimentally obtained spectrum of H₂O, H₂O₂ and peroxo were fitted individually using DM Fit. DM Fit is described by D. Massiot et al. in Magnetic Resonance in Chemistry 2002, vol. 40, pages 70-76.

The initial guess for each component was based on the previously DFT calculated NMR parameters in C. P. Gordon et al., Nature 2020, 586, 708-713, and the lineshape parameters of each species was optimized to converge to a best fit that provides the ratio of each species. Based on said DFT calculations the observed ¹⁷O NMR signal were assigned. The respective concentrations were obtained by multiplication of these ratios with the initial concentration (1.6 M or 1.1 M, respectively) of the aqueous stock solution of H₂¹⁷O₂ that was used for the wet impregnation.

The relative values for H₂¹⁷O₂, bridging μ² η²-peroxo, and H₂¹⁷O (see “rel. H₂O₂”, “rel. μ-peroxo” and “rel. H₂O” in table 1 below) after 15 min and after 2 hours were obtained from the measurements and applying the above quantification protocol.

The concentration of bridging μ² η²-peroxo species in a H₂¹⁷O₂-activated sample was calculated based on the value for the rel. bridging μ² η²-peroxo concentration according to the following calculation:

(concentration of bridging μ² η²-peroxo species)=[(rel. bridging μ² η²-peroxo concentration)×(concentration of H₂¹⁷O₂ solution)×(volume of ¹⁷O-labelled H₂O₂ solution used for wet impregnation)]/[weight of the catalyst sample in mg]

From said concentration the molar concentration of bridging μ² η²-peroxo species per titanium (i.e. mol bridging μ² η²-peroxo per mol Ti) was calculated.

### Reference Example 1.3: X-Ray Powder Diffraction and Determination of the Crystallinity

X-ray diffraction data are collected on a Cuk a Bragg-Brentano Bruker D8 Advance Series II diffractometer. The samples were ground using an IKA Tube Mill with 10000 U/min and then pressed into a standard flat sample holder provided by Bruker AXS GmbH for BraggBrentano geometry data collection. The flat surface was achieved using a glass plate to compress and flatten the sample powder. The angular range is 5°-50° (2θ) with a step width of 0.02° (2θ). Acquisition time is optimised to ensure that the highest intensity signal has at least 200,000 counts. The divergence slit is set to 0.3° opening angle. The total crystallinity is determined using a Rietveld based method as described by I. C. Madsen and N. V. Y. Scarlett, Chapter 11.3.2.1, in Powder Diffraction-Theory and Practice edited by R. E. Dinnebier and S. J. L. Billinge. The method is implemented in the data analysis software TOPAS (TOPAS 6 Users Manual, Bruker AXS GmbH, Karlsruhe, Germany).

To set up the model the instrumental parameters were encoded using the fundamental parameter approach (see TOPAS 6 Users Manual, Bruker AXS GmbH, Karlsruhe, Germany). The structure of a Ti doped MFI zeolite structure as described by Lamberti C., Bordiga S., Zecchina A., Carati A., Fitch A. N., Artioli G., Petrini G., Salvalaggio M., Marra G. L. “Structural Characterization of Ti-Silicalite-1: A Synchrotron Radiation X-Ray Powder Diffraction Study” in J. Catal. 1999, 183, 222-231 is added to the model. The amorphous content is modelled by collecting scattering data on an amorphous silica compound, obtained by drying colloidal silica (Ludox® AS40, dried at 120 °C). Using the approach described by I. C. Madsen and N. V. Y. Scarlett the amorphous content is modelled using a tetragonal lattice with a space group P41212 (92). This selection generates 13 reflections within the scattering range when the lattice parameters a=b=5.07 Å, c=6.909 Å are used. The intensities are fit to the pure material. The crystallite size (LVol IB) of 0.6 ensures a good fit to the broad diffraction signals of amorphous material. For the refinement against data from a real sample the only parameter associated with the amorphous signal which is allowed to vary is that of the scale factor. The linear dependency of the mass fraction to the scattering intensity allows for a one-point calibration. This calibration value is determined and placed in lieu of the unit cell mass. The ZMV calculation then results in the value being reported with the accompanying structures. Crystallinity is then reported in the following manner:

Crystallinity=(Sum of Mass % crystalline material)/(Sum of Mass % crystalline material+amorphous material).

### Reference Example 1.4: Determination of the BET Specific Surface Area

The BET specific surface area was determined via nitrogen physisorption at 77 K according to the method disclosed in DIN ISO 9277 from 2014.

### Reference Example 1.5: Determination of the Propylene Oxide Activity (PO Test)

In the PO test, a preliminary test procedure to assess the possible suitability of the moldings as catalyst for the epoxidation of propylene, the moldings were tested in a steel autoclave by reaction of propylene with an aqueous hydrogen peroxide solution (30 weight-%) to yield propylene oxide. In particular, 0.625 g of the molding were introduced together with 79.2 g of methanol in a steel autoclave. 23 ml of liquid propylene was pressed into the steel autoclave and the steel autoclave was heated to 40 °C. At this temperature, 22.1 g of an aqueous hydrogen peroxide solution (30 weight-% in water) were introduced into the steel autoclave followed by a further 9 g of methanol. After a reaction time of 4 h at 40 °C, the pressure was gently released, and the liquid phase was analyzed by gas chromatography with respect to its propylene oxide content. The propylene oxide content of the liquid phase (in weight-%) was the result of the PO test, i.e. the propylene oxide activity of the molding.

The pressure drop rate was determined following the pressure progression during the PO test described above. The pressure progression was recorded using a S-11 transmitter (from Wika Alexander Wiegand SE & Co. KG), which was positioned in the pressure line of the autoclave, and a graphic plotter Buddeberg 6100A. The respectively obtained data were read out and depicted in a pressure progression curve.

The pressure drop rate (PDR) was determined according to the following equation:

PDR=[p(max)−p(min)]/delta t, with
PDR/(bar/min)=pressure drop rate
p(max)/bar=maximum pressure at the start of the reaction
p(min)/bar=0.9×p(max)
delta t/min=time difference from the start of the reaction to the point in time where p(min) was observed.

### Reference Example 1.6: K-80 Test

The k80-test was designed as a semi-quantitative experiment to assess the rate of decomposition of H₂O₂ by TS-1 and similar titanium containing zeolites. It allows to quantitatively determine the effect of different catalyst treatments on the decomposition of H₂O₂.

### Experimental Procedure

In a clean 50 ml flask with a magnetic stirring bar and a thermometer were placed 30 g of deionised water and 6.5 g of a 40 weight-% hydrogen peroxide solution at room temperature (22 °C). At this point a t=0 probe of ca. 0.3 ml was taken with a pipette. The flask was lightly stoppered and then immersed in a previously equilibrated thermostating bath set to 80 °C. To obtain reproducible results it was important to use always the same amount of catalyst and to control the temperature to better than +1 °C during the experiment. As soon as the hydrogen peroxide solution was in thermal equilibrium with the thermostating bath, 500 mg (+1 mg) of the catalyst (either powder or moldings (preferably extrudates)) were added. The suspension was stirred, and samples of the supernatant liquid were then taken at regular intervals. The probe should be taken with a 1 ml syringe with a one-way filter Millipore Millex-HV SLHV 013 NL or equivalent. First 0.6 ml of solution were sucked into the syringe through the filter. Then 0.3 ml of the solution were through the filter into the flask. This was necessary in order to minimise loss of catalyst. The remaining 0.3 ml in the syringe were then used for the peroxide determination. The interval between probes was usually between 30 and 60 min depending on the catalyst activity.

The experiment was finished after 7 hours.

### Analytics

The probes were analysed for H₂O₂ content by using a standard cerimetric titration. It was advisable to analyse the probes as soon as possible after they are collected. In order to ensure a good precision, the amount of titrating solution used should be at least 5 ml. If necessary, a larger amount of probe had to be weighed in.

### Data Analysis

The natural logarithm of the H₂O₂ concentration was plotted against time. Using least squares methods, the slope is extracted. This slope was the pseudo-first order decay rate of H₂O₂ in the presence of the catalyst (in h⁻¹) and is called the k80 value.

### Reference Example 1.7: Determination of the Crush Strength

The crush strength as referred to in the context of the present invention is to be understood as having been determined via a crush strength test machine Z2.5/TS1S, supplier Zwick GmbH & Co., D-89079 Ulm, Germany. As to fundamentals of this machine and its operation, reference is made to the respective instructions handbook “Register 1: Betriebsanleitung/Sicherheitshandbuch für die Material-Prüfmaschine Z2.5/TS1S”, version 1.5, December 2001 by Zwick GmbH & Co. Technische Dokumentation, August-Nagel-Strasse 11, D-89079 Ulm, Germany. The machine was equipped with a fixed horizontal table on which the molding (preferably strand) was positioned, with the longitudinal axis of the molding (preferably strand) parallel to the horizontal table. A plunger with rectangular surface, having a width of 3 mm and depth of 10 mm, which was freely movable in vertical direction actuated the molding (preferably strand) against the fixed table. The apparatus was operated with a preliminary force of 0.5 N, a shear rate under preliminary force of 10 mm/min and a subsequent testing rate of 1.6 mm/min. The vertically movable plunger was connected to a load cell for force pick-up and, during the measurement, moved toward the fixed turntable on which the molding (preferably strand) to be investigated is positioned, thus actuating the molding (preferably strand) against the table. The plunger was applied to the moldings (preferably strands) with the short edge of the plunger perpendicularly to the longitudinal axis of the moldings (preferably strands). With said machine, a given molding (preferably strand) as described below was subjected to an increasing force via the plunger until a break occurs. The force for breaking was referred to as the crushing strength of the molding (preferably strand).

Controlling the experiment was carried out by means of a computer which registered and evaluated the results of the measurements. The values obtained were the mean value of the measurements for 25 moldings (preferably strands) in each case. Thus, the obtained mean values are also referred to herein as average crush strength.

### Reference Example 1.8: Determination of N₂ Adsorption/Desorption Isotherm

The nitrogen adsorption/desorption isotherm was determined at 77 K according to the method disclosed in DIN ISO 9277 from 2014.

### Reference Example 1.9: Determination of the total pore volume

The total pore volume was determined via intrusion mercury porosimetry according to DIN 66133 from 1993.

### Reference Example 1.10: Determination of activation factor

An activation factor was determined for zeolitic materials as well as catalyst moldings being prepared in accordance with the present invention as well as for zeolitic materials and catalyst moldings not in accordance with the present invention. To this effect, the water adsorption (W), preferably determined according to Reference Example 1.1, and the concentration (C) of bridging μ² η²-peroxo species per Ti in the H₂O₂-activated zeolitic material, as determined by quantitative ¹⁷O NMR spectroscopy, preferably determined according to Reference Example 1.2, were determined. The activation factor (A) was calculated based thereon according to formula I, wherein in accordance with formula I, the activation factor is the multiplication product of the water adsorption and the concentration of bridging μ² η²-peroxo species per Ti in the H₂O₂-activated zeolitic material:

A=W×C (I).

### Reference Example 1.11: Determination of Bulk Density

The bulk density is measured by filling a 150 g sample of mixed moldings (preferably extrudates) in a graded glass cylinder having an inner diameter of 50 mm and height of 340 mm with a filling time of 5 s, and measuring the volume occupied by the moldings (preferably extrudates). The bulk density is then obtained by dividing the mass of material filled (g) in the cylinder to the volume of the bed (mL).

### Reference Example 1.12: Determination of Aspect Ratio

The catalyst moldings (preferably strands) were scattered on a black substrate uniformly and thus formed a monolayer. By using light microscopy (bright field contrast) several images were stitched together to form an image with 55.68 mm×56.52 mm (3966×4026 pxls). By using the “Particle Sizer”-Plugin of ImageJ (Caroline A Schneider, Wayne S Rasband and Kevin W Eliceiri: NIH Image to ImageJ: 25 years of image analysis in Nature Methods, volume 9, 2012, p. 671-675; doi: 10.1038/nmeth.208) for segmentation of the moldings (preferably strands), the ratio of the long (larger) side length of a minimum bounding rectangle and the maximum inscribed circle diameter was calculated as the aspect ratio for each molding (preferably strand).

### Reference Example 1.13: Determination of X-Ray Photoelectron Spectroscopy (XPS)

XPS analyses were carried out with a Phi Versa Probe 5000 spectrometer (Ulvac PHI, MI) using monochromatic Al Kα radiation (49.9 W). The XPS system was calibrated according to ISO 15472.2001. The binding energy (B.E.) of Au 4f7/2 is 84.00 eV and that of Cu2p3/2 is 932.62 eV.

All samples were mounted insulated against ground and neutralized in the course of the measurements with the built-in charge neutralizer and measured on three non-overlapping sample positions using a spot size of 200 μm×200 μm with a pass energy of 117 eV and an energy step size of 0.5 eV. High resolution analyses were carried out on the same analysis area with a pass energy of 23.5 eV and an energy step size of 0.1 eV. Total dwell time per energy step was 6600 ms for Ti 2p, 5500 ms for C 1s, 3300 ms for Si 2p, 1100 ms for the survey spectrum.

Spectra have been charge corrected to the position of Extra-Framework TiO₂ at 458.6 eV as described by Langerame, F., Salvi, A. M., Silletti, M. and Moretti, G. (2008) “XPS characterization of a synthetic Ti-containing MFI zeolite framework: the titanosilicalites, TS-1” in Surf. Interface Anal., 40:695-699; https://doi.org/10.1002/sia.2739.

All Spectra were analyzed using standard XPS-analysis software, namely CasaXPS version 2.3.26rev1.11 as described by Fairley N, (2011); CASA-XPS, 2.3.25ed., Casa Software Ltd. using Shirley background subtraction of the main peaks for the elements of interest with a background averaging of 20 (Average Width). Relative sensitivity factors and transmission function as provided by the instrument manufacturer were used for quantification.

The C 1s signals were fitted with four lines.

| | | B.E. | Position | Width | FWHM | |
|---|---|---|---|---|---|---|
| | Species | (eV) | Constraint (eV) | (eV) | constaint | LineShape |
| A | CH, CC | 284.4 | +−0.1 | 1.9 eV | +−0.1 | LA(1, 1, 900) |
| | (Hydrocarbon) | | | | | |
| B | C—O (Hydroxyl) | | A + 1.5 eV | | A*1 | LA(1, 1, 900) |
| C | C═O (Carbonyl) | | A + 3 | | A*1 | LA(1, 1, 900) |
| D | COOR | | A + 4.2 | | A*1 | LA(1, 1, 900) |
| | (Carboxylate) | | | | | |

The Ti 2p signals were fitted with two doublets representing extraframework Ti and Framework Ti according to Langerame, F., et al.

| | | B.E | Constraint | Width | FWHM | | Area |
|---|---|---|---|---|---|---|---|
| | Species | (eV) | (eV) | (eV) | constaint | LineShape | Constraint |
| A | Extraframework | 458.6 | +−0.1 | 1.8 +− 0.2 | | LF(0.65, 0.65, 200, 900) | |
| | Ti (Ti 2p3/2) | | | | | | |
| B | Extraframework | | A + 5.74 | | A*1.5 | LF(0.9, 0.85, 200, 900) | A*0.45 |
| | Ti (Ti 2p1/2) | | | | | | |
| C | Framework Ti | 460.0 | +−0.2 | 1.6 +− 0.2 | | LF(0.65, 0.65, 200, 900) | |
| | (Ti 2p3/2) | | | | | | |
| D | Framework Ti | | C + 5.74 | | C*1.5 | LF(0.9, 0.85, 200, 900) | C*0.45 |
| | (Ti 2p1/2) | | | | | | |

Relative sensitivity factors as provided by the instrument manufacturer were used for quantification.

### Reference Example 2: Preparation of a Zeolitic Material Having Framework Type MFI (TS-1))

### Reference Example 2.1: Preparation of a Zeolitic Material Having Framework Type MFI Wherein from 98 to 100 Weight-% of the Zeolitic Material Consist of Ti, Si, O, and H (Titanium Silicalite-1 (TS-1))

A titanium silicalite-1 (TS-1) powder was prepared according to the following recipe: 500 g TEOS (tetraethyl orthosilicate) and 15 g TEOTi (tetraethyl orthotitanate) were loaded into a four-neck flask at room temperature and stirring (200 rpm) was started. Then, 220 g of an aqueous solution comprising 40 weight-% TPAOH (tetrapropylammonium hydroxide) and 300 g of deionized water were added. The pH of the resulting solution was 14.21, determined with a pH sensitive glass electrode. Stirring was continued for 60 min, whereby the temperature of the mixture rose to 60 °C. Ethanol released by hydrolysis was separated by distillation at a bottoms temperature of 95 °C, obtaining about 540 g ethanol and a gel. The pH of the gel was 12.34, determined with a pH sensitive glass electrode. Subsequently, the gel was cooled down to 40 °C under stirring and 540 g of deionized water were added. The pH of the obtained mixture was 12.01, determined with a pH sensitive glass electrode. Crystallization was performed in an autoclave under stirring at 175 °C within 16 h and 20 min at autogenous pressure.

The obtained suspension was worked-up as follows. The suspension was diluted 1:1 with deionized water under stirring (200 rpm, Teflon anchor stirrer) and precipitated with 10% HNO₃ (approx. 130 g) at pH 7.31, determined with a pH sensitive glass electrode, and filtered off through a porcelain suction filter (blue belt filter). The filter cake was washed 3 times with 1000 ml deionized water, dried in an oven for 4 h at 120 °C and calcined for 5 h at 490 °C (heating rate 2 °C/min) in air.

The yield was 148 g. The resulting powder had a total organic carbon content (TOC) of 0.02 g/100 g, determined according to DIN EN 1484, a potassium content of less than 0.01 g/100 g, a sodium content of 0.01 g/100 g, a Si content of 43 g/100 g, and a Ti content of 1.9 g/100 g. As determined by X-ray diffraction analysis according to Reference Example 1.3, the sample had a crystallinity of 91% and essentially consisted of TS-1 (100 weight-% of crystalline TS-1).

### Reference Example 2.2: Water Treatment of a Zeolitic Material Having Framework Structure Type MFI (TS-1)

750.0 g deionized water and 300 g of an aqueous solution comprising 40 weight-% tetrapropylammonium hydroxide were provided in a beaker. Then, 140 g of a TS-1 powder prepared according to Reference Example 2.1 were added under stirring (200 rpm, anchor stirrer). This mixture was stirred for 60 min, and then transferred in an autoclave. The mixture was then heated to 170 °C under stirring and stirred for 84 h at 170 °C under autogenous pressure. The obtained suspension was worked-up as follows. The suspension was filled into beakers and centrifuged for 60 min with 4000 rpm. The solids were dried in an oven for 10 h at 120 °C and then calcined for 5 h at 490 °C (heating rate 2 °C/min) in air.

The yield was 121 g. The resulting powder had a Ti content of 1.8 g/100 g, a water adsorption of 6.3 weight-% determined according to Reference Example 1.1 and showed a BET specific surface area of 443 m²/g determined as described in Reference Example 1.4. Further, the resulting powder exhibited a type IV nitrogen adsorption/desorption isotherm determined as described in Reference Example 1.8.

### Example 1: Preparing a Molding According to the Invention

### Example 1.1: Shaping of a Zeolitic Material Having Framework Structure Type MFI (TS-1)

100.0 g of the zeolitic material of Reference Example 2.2 and 4.0 g Walocel™ (Walocel MW 15000 GB, Wolff Cellulosics GmbH & Co. KG, Germany) were provided in a kneader and mixed for 5 minutes. Then, 111.0 g of polystyrene (33 wt.-% aqueous dispersion) were added, and the resulting mixture kneaded for 10 minutes. Then, 1.33 g of polyethylene oxide (PEO) were added, and the resulting mixture kneaded for 10 minutes. Then, 83.3 g of an aqueous suspension comprising 40 weight-% colloidal silica (Ludox® AS 40) were added and the resulting mixture kneaded for 10 minutes.

Subsequently, 25.0 g deionized water were added, and the resulting mixture kneaded for 15 minutes.

After that, the kneaded mass was subjected to shaping. For shaping, the kneaded mass was extruded at a pressure of 150 bar (abs) to give strands with a circular cross-sectional profile having a diameter of 1.9 mm. The strands were then dried in an oven for 4 h at 120 °C and then calcined in air for 5 h at 490 °C (heating rate: 2 °C/min).

The yield was 124 g. The obtained material had a water adsorption of 6.7 weight-% determined according to Reference Example 1.1, a bulk density of 332 g/l, a Ti content of 1.3 g/100 g, a BET specific surface area of 362 m²/g determined as described in Reference Example 1.4, an average crush strength of 2.9 N determined according to Reference Example 1.7 a total pore volume of 0.96 ml/g determined as described in Reference Example 1.9. As determined by X-ray diffraction analysis, the sample had a crystallinity of 58% and essentially consisted of TS-1 (0.7 weight-% crystalline anatase and 99.3 weight-% of crystalline TS-1).

As determined according to Reference Example 1.12 and displayed in FIG. 4, 7% of the catalyst moldings have an aspect ratio in the range of from 1.0 to 1.5, 18% have an aspect ratio in the range of greater than 1.5 to 2.0, 16% have an aspect ratio in the range of greater than 2.0 to 2.5, 37% have an aspect ratio in the range of greater than 2.5 to 3.0, 10% have an aspect ratio in the range of greater than 3.0 to 3.5, 7% have an aspect ratio in the range of greater than 3.5 to 4.0, 3% have an aspect ratio in the range of greater than 4.0 to 4.5, and 3% have an aspect ratio greater than 4.5.

### Example 1.2: Water Treatment of Shaped TS-1

50.0 g of the strands prepared according to Example 1.1 were loaded into an autoclave. Then, 750 g deionized water were added. The resulting mixture was heated to a temperature of 145 °C for 8 h in an autoclave. Thereafter, the obtained water-treated strands were separated and sieved over a 800 μm sieve. The obtained strands were then washed with deionized water and dried in an oven for 4 h at 120 °C (heating rate: 2 °C/min) in air and then calcined for 2 h at 450 °C (heating rate: 2 °C/min) in air.

The resulting material had a Ti content of 1.4 g/100 g, a total pore volume of 1.0 ml/g determined according to Reference Example 1.11, a BET specific surface area of 323 m²/g determined as described in Reference Example 1.4, an average crush strength of 8.6 N determined according to Reference Example 1.7, a water adsorption of 4.5 weight-% determined according to Reference Example 1.1, and a bulk density of 332 g/l. As determined by X-ray diffraction analysis according to Reference Example 1.3, the sample had a crystallinity of 51%.

As determined according to Reference Example 1.12 and displayed in FIG. 5, 3% of the catalyst moldings have an aspect ratio in the range of from 1.0 to 1.5, 17% have an aspect ratio in the range of greater than 1.5 to 2.0, 24% have an aspect ratio in the range of greater than 2.0 to 2.5, 24% have an aspect ratio in the range of greater than 2.5 to 3.0, 21% have an aspect ratio in the range of greater than 3.0 to 3.5, 3% have an aspect ratio in the range of greater than 3.5 to 4.0, 3% have an aspect ratio in the range of greater than 4.0 to 4.5, and 3% have an aspect ratio greater than 4.5.

### Reference Example 3: Preparing a Zeolitic Material Having Framework Structure MFI

Using a modified synthetic procedure, a TS-1 material was synthesized having a Ti content of 1.2 g/100 g, a crystallinity of 89%, a BET specific surface area of 392 m²/g determined according to Reference Example 1.4, a water adsorption of 4.4 weight-% determined according to Reference Example 1.1 and exhibiting a type IV nitrogen adsorption/desorption isotherm determined as described in Reference Example 1.8.

### Example 2: Preparing a Molding According to the Invention

100.0 g of the zeolitic material of Reference Example 3 and 4.0 g Walocel™ (Walocel MW 15000 GB, Wolff Cellulosics GmbH & Co. KG, Germany) were provided in a kneader and mixed for 5 minutes. Then, 111.0 g of polystyrene (33 wt.-% aqueous dispersion) were added, and the resulting mixture kneaded for 10 minutes. Then, 1.33 g of polyethylene oxide (PEO) were added, and the resulting mixture kneaded for 10 minutes. Then, 83.3 g of an aqueous suspension comprising 40 weight-% colloidal silica (Ludox® AS 40) were added and the resulting mixture kneaded for 10 minutes.

Subsequently, 25.0 g deionized water were added, and the resulting mixture kneaded for 15 minutes.

After that, the kneaded mass was subjected to shaping. For shaping, the kneaded mass was extruded at a pressure of 150 bar (abs) to give strands with a circular cross-sectional profile having a diameter of 1.9 mm. The strands were then dried in an oven for 4 h at 120 °C and then calcined in air for 5 h at 490 °C (heating rate: 2 °C/min).

The resulting strands had a Ti content of 0.9 g/100 g, a BET specific surface area of 353 m²/g determined according to Reference Example 1.4, a total pore volume of 0.97 ml/g determined according to Reference Example 1.11, a water adsorption of 4.6 weight-% determined according to Reference Example 1.1, an average crush strength of 3.5 N determined according to Reference Example 1.7, and a bulk density of 374 g/l.

As determined by X-ray diffraction analysis according to Reference Example 1.3, the sample had a crystallinity of 67%.

As determined according to Reference Example 1.12 and displayed in FIG. 6, 19% of the catalyst moldings have an aspect ratio in the range of from 1.0 to 1.5, 43% have an aspect ratio in the range of from greater than 1.5 to 2.0, 24% have an aspect ratio in the range of from greater than 2.0 to 2.5, 10% have an aspect ratio in the range of from greater than 2.5 to 3.0, and 4% have an aspect ratio in of greater than 3.0.

### Example 3: Preparing a Molding According to the Invention

120.0 g of the zeolitic material of Reference Example 3 and 4.8 g Sesbania cannabina powder (Dongying Jing Xiang Sesbania Powder Co., LTD) were provided in a kneader and mixed for 5 minutes. Then, 61.5 g of an aqueous suspension comprising 40 weight-% colloidal silica (Ludox® AS 40) were added and the resulting mixture was kneaded for 10 minutes.

Subsequently, 95.0 g deionized water were added, and the resulting mixture kneaded for 35 minutes.

After that, the kneaded mass was subjected to shaping. For shaping, the kneaded mass was extruded at a pressure of 140 bar (abs) to give strands with a circular cross-sectional profile having a diameter of 1.9 mm. The strands were then dried in an oven for 4 h at 120 °C and then calcined in air for 5 h at 490 °C (heating rate: 2 °C/min).

The yield was 127 g.

The resulting strands had a Ti content of 1.0 g/100 g, a BET specific surface area of 380 m2/g determined according to Reference Example 1.4, a total pore volume of 0.74 ml/g determined according to Reference Example 1.9, a water adsorption of 5.7 weight-% determined according to Reference Example 1.1, an average crush strength of 6.0 N determined according to Reference Example 1.7, and a bulk density of 374 g/l.

As determined by X-ray diffraction analysis according to Reference Example 1.3, the sample had a crystallinity of 75%.

As determined according to Reference Example 1.12 and displayed in FIG. 7, 18% of the catalyst moldings have an aspect ratio in the range of from 1.0 to 1.5, 18% have an aspect ratio in the range of greater than 1.5 to 2.0, 16% have an aspect ratio in the range of greater than 2.0 to 2.5, 14% have an aspect ratio in the range of greater than 2.5 to 3.0, 12% have an aspect ratio in the range of greater than 3.0 to 3.5, 8% have an aspect ratio in the range of greater than 3.5 to 4.0, 6% have an aspect ratio in the range of greater than 4.0 to 4.5, 4% have an aspect ratio in the range of greater than 4.5 to 5.0, 3% have an aspect ratio in the range of greater than 5.0 to 5.5, and 1% have an aspect ratio greater than 5.5.

### Reference Example 4: Preparing a Zeolitic Material Having Framework Structure MFI

A TS-1 zeolite was prepared in accordance with Example 1 of WO 2011/064191 A1 with the exception that 10 weight-% of tetraethyl orthosilicate were used as binder based on 100 weight-% of the TS-1 material.

1072 g de-ionized water were provided in a beaker. Then, 424 g tetrapropylammonium hydroxide (as an aqueous solution comprising 40 weight-% tetrapropylammonium hydroxide) were added under stirring. Subsequently, 200 g of the TS-1 zeolite were added. This mixture was homogenized for 30 min. The mixture was then transferred in an autoclave, where it was hydrothermally treated at 170 °C for 24 hours. The resulting solids were separated via centrifugation, and the solid residue obtained was washed with deionized water. The resulting solid material was heated in air within 60 min to a temperature of 110 °C and dried at said temperature for 4 h. Then, the resulting solid material was heated in air within 190 min to a temperature of 520 °C and calcined at said temperature for 16 h.

The thus obtained TS-1 material had a Si content of 45 weight-%, a Ti content of 1.7 weight-% and a total organic carbon content (TOC) of less than 0.1 weight-%. The BET specific surface area was 450 m²/g.

5000 g of aqueous nitric acid (10 weight-% HNO₃ in water) were provided in a glass beaker. Under stirring, 250 g of the TS-1 material were added thereto. The resulting suspension—while being stirred at 250 rpm—was refluxed at 100 °C for 1 hour. For work-up, the resulting solids were separated via centrifugation. The resulting solid material was heated in air within 60 min to a temperature of 120 °C and dried at said temperature for 4 h. Then, the resulting solid material was heated in air within 190 min to a temperature of 500 °C and calcined at said temperature for 5 h.

The thus obtained TS-1 material had a Si content of 45 weight-%, a Ti content of 1.8 weight-% and a total organic carbon content (TOC) of less than 0.1 weight-%. The BET specific surface area was 453 m²/g, determined according to Reference Example 1.4, and the water adsorption 7.0 wt.-%, determined according to Reference Example 1.1. The crystallinity was 97%, and about 1% of anatase were detectable by X-ray diffraction, determined according to Reference Example 1.3. Further, the TS-1 material exhibited a type IV nitrogen adsorption/desorption isotherm determined as described in Reference Example 1.8.

### Example 4: Preparing a Catalyst Molding

50 g of the zeolitic material of Reference Example 4 and 2 g Walocel™ (Walocel MW 15000 GB, Wolff Cellulosics GmbH & Co. KG, Germany) were provided in a kneader and kneaded for 5 minutes. Then, 50.4 g of polystyrene (33 wt.-% aqueous dispersion) were added. After 10 minutes, 0.67 g polyethylene oxide (PEO, Union Carbide, PolyOX Coagulant) were added, and the mixture was kneaded. After further 10 minutes, 41.65 g of a colloidal silica (Ludox® AS 40) were added. Subsequently, the addition of de-ionized water was started in portions of 10 ml every 10 minutes to result in a total addition of water of 100 mL. The total kneading time was 45 minutes. After completion of the water addition, the kneaded mass was subjected to shaping. For shaping, the kneaded mass was extruded at a pressure of 150 bar (abs) to give strands with a circular cross-sectional profile having a diameter of 1.7 mm. The strands were then dried and calcined in air according to the following program:
1. heating within 60 minutes to a temperature of 120 °C;
2. keeping the temperature of 120 °C for 4 h;
3. heating within 185 minutes to a temperature of 490 °C;
4. keeping the temperature of 490 °C for 5 h.

The yield was 55 g.

36 g of the obtained strands were mixed in four portions of each 9 g with 180 g deionized water per portion. The resulting mixtures were heated to a temperature of 145 °C for 8 h in an autoclave. Thereafter, the obtained water-treated strands were separated and was sieved over a 0.8 mm sieve. The obtained water-treated strands were then washed with deionized water and pre-dried in a stream of nitrogen at ambient temperature. The washed and pre-dried strands were subsequently dried and calcined in air according to the following program:
1. heating within 60 minutes up to 120 °C;
2. keeping the temperature of 120 °C for 4 h;
3. heating within 165 minutes up to 450 °C;
4. keeping the temperature of 450 °C for 2 h.

The yield was 36.2 g. The resulting material had a total organic carbon content (TOC) of less 0.1 g/100 g, a Si content of 45 g/100 g, and a Ti content of 1.3 g/100 g. The hardness of the strands determined according to Reference Example 1.7 was 4.3 N, and the total pore volume determined according to Reference Example 1.9 was 0.82 ml/g.

### Reference Example 5: Preparing a TS-1 Zeolitic Material

For the gel preparation, 500 g tetraethylorthosilicate (TEOS) and 15 g tetraethylorthotitanate (TEOTi; Merck) were filled into a beaker. Then, a solution of 300 g de-ionized water and 220 g aqueous tetrapropylammonium hydroxide (TPAOH; 40 weight-% in water) was added under stirring (200 rpm). The resulting mixture had a pH of 13.5, determined with a pH sensitive glass electrode. The mixture was hydrolyzed at room temperature for 60 min during which the temperature rose to 60 °C. The mixture had a pH of 12.6, determined with a pH sensitive glass electrode, then. Afterwards the ethanol was distilled off until the sump reached a temperature of 95 °C. 540 g of distillate was obtained from distillation.

The synthesis gel was then cooled to 40 °C under stirring and 542 g de-ionized water added thereto. The resulting mixture had a pH of 11.9, determined with a pH sensitive glass electrode.

The synthesis gel was then transferred into an autoclave. The synthesis gel was heated under stirring in the autoclave to a temperature of 175 °C and stirred at said temperature for 16 h under autogenous pressure. The pressure was in the range of from 8.4 to 10.9 bar (abs). The resulting suspension was then worked-up. To this effect, the resulting suspension was diluted with de-ionized water, wherein the weight ratio of the suspension to de-ionized water was 1:1. Then, about 164 g nitric acid (10 weight-% in water) were added and the resulting mixture had a pH of 7.35, determined with a pH sensitive glass electrode. The obtained solids were filtered off and washed four times with de-ionized water (each time 1000 ml de-ionized water were used). Subsequently, the solids were dried in an oven in air at 120 °C for 16 h and then calcined in air at 490 °C for 5 h, wherein the heating rate for calcining was 2 °C/min.

The thus obtained TS-1 material had a Si content of 43 weight-%, a Ti content of 2.0 weight-% and a total organic carbon content (TOC) of less than 0.1 weight-%. The BET specific surface area was 457 m²/g, determined according to Reference Example 1.4, and the water adsorption 11.5 wt.-%, determined according to Reference Example 1.1. The crystallinity was 88% as determined by X-ray diffraction according to Reference Example 1.3.

### Example 5: Preparing a Catalyst Molding

100.0 g of the zeolitic material of Reference Example 5 and 4.0 g Walocel™ (Walocel MW 15000 GB, Wolff Cellulosics GmbH & Co. KG, Germany) were provided in a kneader and mixed for 5 minutes. Then, 111.0 g of polystyrene (33 wt.-% aqueous dispersion) were added, and the resulting mixture kneaded for 10 minutes. Then, 1.33 g of polyethylene oxide (PEO) were added, and the resulting mixture kneaded for 10 minutes. Then, 83.3 g of an aqueous suspension comprising 40 weight-% colloidal silica (Ludox® AS 40) were added and the resulting mixture kneaded for 10 minutes.

Subsequently, 25.0 g deionized water were added, and the resulting mixture kneaded for 15 minutes.

After that, the kneaded mass was subjected to shaping. For shaping, the kneaded mass was extruded at a pressure of 150 bar (abs) to give strands with a circular cross-sectional profile having a diameter of 2.0 mm. The strands were then dried in an oven for 4 h at 120 °C and then calcined in air for 5 h at 490 °C (heating rate: 2 °C/min).

50.0 g of the obtained strands were loaded into an autoclave. Then, 750 g deionized water were added. The resulting mixture was heated to a temperature of 145 °C for 8 h in an autoclave. Thereafter, the obtained water-treated strands were separated and sieved over an 800 μm sieve. The obtained strands were then washed with deionized water and dried in an oven for 4 h at 120 °C (heating rate: 2 °C/min) in air and then calcined for 2 h at 450 °C (heating rate: 2 °C/min) in air.

As determined according to Reference Example 1.12 and displayed in FIG. 8, 59% of the catalyst moldings have an aspect ratio in the range of from 1.0 to 1.5, 34% have an aspect ratio in the range of from greater than 1.5 to 2.0, and 7% have an aspect ratio of greater than 2.0.

The resulting strands had a Ti content of 1.6 g/100 g, a BET specific surface area of 315 m2/g determined according to Reference Example 1.4, a total pore volume of 0.9 ml/g determined according to Reference Example 1.9, a water adsorption of 6.05 weight-% determined according to Reference Example 1.1, an average crush strength of 5.6 N determined according to Reference Example 1.7, and a bulk density of 411 g/l.

As determined by X-ray diffraction analysis, the sample had a crystallinity of 61%, determined according to Reference Example 1.3.

### Comparative Example 6: Preparing a Catalyst Molding

100.0 g of the zeolitic material of Reference Example 5 and 4.0 g Walocel™ (Walocel MW 15000 GB, Wolff Cellulosics GmbH & Co. KG, Germany) were provided in a kneader and mixed for 5 minutes. Then, 111.0 g of polystyrene (33 wt.-% aqueous dispersion) were added, and the resulting mixture kneaded for 10 minutes. Then, 1.33 g of polyethylene oxide (PEO) were added, and the resulting mixture kneaded for 10 minutes. Then, 83.3 g of an aqueous suspension comprising 40 weight-% colloidal silica (Ludox® AS 40) were added and the resulting mixture kneaded for 10 minutes.

Subsequently, 25.0 g deionized water were added, and the resulting mixture kneaded for 15 minutes.

After that, the kneaded mass was subjected to shaping. For shaping, the kneaded mass was extruded at a pressure of 150 bar (abs) to give strands with a circular cross-sectional profile having a diameter of 2.0 mm. The strands were then dried in an oven for 4 h at 120 °C and then calcined in air for 5 h at 490 °C (heating rate: 2 °C/min).

The resulting strands had a Ti content of 1.6 g/100 g, a BET specific surface area of 373 m2/g determined according to Reference Example 1.4, a total pore volume of 0.88 ml/g determined according to Reference Example 1.9, a water adsorption of 10.41 weight-% determined according to Reference Example 1.1, an average crush strength of 1.3 N determined according to Reference Example 1.7, and a bulk density of 422 g/l.

As determined by X-ray diffraction analysis, the sample had a crystallinity of 59%, determined according to Reference Example 1.4.

As determined according to Reference Example 1.12 and displayed in FIG. 9, 61% of the catalyst moldings have an aspect ratio in the range of from 1.0 to 1.5, 31% have an aspect ratio in the range of from greater than 1.5 to 2.0, and 8% have an aspect ratio of greater than 2.0.

### Example 7: Preparing a Molding According to the Invention

100.0 g of the zeolitic material of Reference Example 3 and 4.0 g Walocel™ (Walocel MW 15000 GB, Wolff Cellulosics GmbH & Co. KG, Germany) were provided in a kneader and mixed for 5 minutes. Then, 111.0 g of polystyrene (33 wt.-% aqueous dispersion) were added, and the resulting mixture kneaded for 10 minutes. Then, 1.33 g of polyethylene oxide (PEO) were added, and the resulting mixture kneaded for 10 minutes. Then, 83.3 g of an aqueous suspension comprising 40 weight-% colloidal silica (Ludox® AS 40) were added and the resulting mixture kneaded for 10 minutes.

Subsequently, 25.0 g deionized water were added, and the resulting mixture kneaded for 15 minutes.

After that, the kneaded mass was subjected to shaping. For shaping, the kneaded mass was extruded at a pressure of 150 bar (abs) to give strands with a circular cross-sectional profile having a diameter of 1.9 mm. The strands were then dried in an oven for 4 h at 120 °C and then calcined in air for 5 h at 490 °C (heating rate: 2 °C/min).

50.0 g of the obtained strands were loaded into an autoclave. Then, 750 g deionized water were added. The resulting mixture was heated to a temperature of 145 °C for 8 h in an autoclave. Thereafter, the obtained water-treated strands were separated and sieved over a 800 μm sieve. The obtained strands were then washed with deionized water and dried in an oven for 4 h at 120 °C (heating rate: 2 °C/min) in air and then calcined for 2 h at 450 °C (heating rate: 2 °C/min) in air.

### Example 8: Preparing a Molding According to the Invention

4,000 g of the zeolitic material of Reference Example 3, 160 g of a cellulose derivative (Zusoplast C39), and 160 g of a polyvinylacetate compound (Optapix PAC 60) were provided in a kneader, and the resulting mixture kneaded for 5 minutes. Then, 2,050 g of an aqueous suspension comprising 40 weight-% colloidal silica (Ludox® AS 40) were added and the resulting mixture kneaded for 10 minutes. Subsequently, 2,000 g deionized water were added, and the resulting mixture kneaded for 10 minutes. Then, 40 g of polyethylene oxide (PEO) were added, and the resulting mixture kneaded for 10 minutes. Subsequently, 400 g deionized water were added, and the resulting mixture kneaded for 10 minutes.

After that, the kneaded mass was subjected to shaping. For shaping, the kneaded mass was extruded at a pressure of 180 bar (abs) to give strands with a circular cross-sectional profile having a diameter of 1.9 mm. The strands were then dried in an oven for 10 h at 120 °C and then calcined in air for 5 h at 500 °C (heating rate: 2 °C/min).

The obtained material had a Ti content of 1.0 g/100 g, a Si content of 46 g/100 g, a total organic carbon content (TOC) of less than 0.06 g/100 g, a water adsorption of 4.9 weight-% determined according to Reference Example 1.1, an average crush strength of 6.43 N determined according to Reference Example 1.7, a bulk density of 409 g/l, a BET specific surface area of 382 m²/g determined according to Reference Example 1.4, and a total pore volume of 0.78 ml/g determined as described in Reference Example 1.9. As determined by X-ray diffraction analysis, the sample had a crystallinity of 67% and essentially consisted of TS-1 (greater than 99 weight-% of crystalline TS-1).

### Example 9: Preparing a Molding According to the Invention

120.0 g of the zeolitic material of Reference Example 3 were provided in a kneader, 4.80 g Sesbania cannabina powder (Dongying Jing Xiang Sesbania Powder Co., LTD) comprising galactomannan were added and the resulting mixture kneaded for 5 minutes. Then, 61.5 g of an aqueous suspension comprising 40 weight-% colloidal silica (Ludox® AS 40) were added and the resulting mixture kneaded for 10 minutes. Subsequently, 80 g deionized water were added, and the resulting mixture kneaded for 10 minutes. Then, 1.20 g of polyethylene oxide (PEO) were added, and the resulting mixture kneaded for 10 minutes. Then, 30 g deionized water were added, and the resulting mixture kneaded for 15 minutes.

After that, the kneaded mass was subjected to shaping. For shaping, the kneaded mass was extruded at a pressure of 150 bar (abs) to give strands with a circular cross-sectional profile having a diameter of 1.9 mm. The strands were then dried in an oven for 4 h at 120 °C and then calcined in air for 5 h at 500 °C (heating rate: 2 °C/min).

The yield was 125.4 g. The obtained material had a Ti content of 0.97 g/100 g, a Si content of 45 g/100 g, a C content of less than 0.01 g/100 g, a water adsorption of 4.9 weight-% determined according to Reference Example 1.1, a bulk density of 366 g/l, an average crush strength of 11.29 N determined according to Reference Example 1.7, and a total pore volume of 0.87 ml/g determined as described in Reference Example 1.9. As determined by X-ray diffraction analysis according to Reference Example 1.3, the sample had a crystallinity of 67% and essentially consisted of TS-1 (greater than 99 weight-% of crystalline TS-1).

### Example 10: Preparing a Molding According to the Invention

100 g of the TS-1 zeolite prepared according to Reference Example 2.2 were loaded into a kneader, 4.00 g of Sesbania cannabina powder (Dongying Jing Xiang Sesbania Powder Co., LTD) comprising galactomannan were added and the resulting mixture kneaded for 5 minutes. Then, 51.25 g of an aqueous suspension comprising 40 weight-% colloidal silica (Ludox® AS 40) were added and the resulting mixture kneaded for 10 minutes. Then, 50 g deionized water were added, and the resulting mixture kneaded for 10 minutes. Then, 20 g deionized water were further added, and the resulting mixture kneaded for 5 minutes. Then, 10 g deionized water were further added, and the resulting mixture kneaded for 5 minutes. Then, 5 g deionized water were further added, and the resulting mixture kneaded for 15 minutes.

After that, the kneaded mass was subjected to shaping. For shaping, the kneaded mass was extruded at a pressure of 90 bar (abs) to give strands with a circular cross-sectional profile having a diameter of 1.7 mm. The strands were then dried in an oven for 4 h at 120 °C and then calcined in air for 5 h at 500 °C (heating rate: 2 °C/min).

The obtained material had a Ti content of 1.6 g/100 g, a Si content of 46 g/100 g, a C content of 0.01 g/100 g, a bulk density of 369 g/l, an average crush strength of 5.2 N determined according to Reference Example 1.7, a water adsorption of 8.0 weight-% determined according to Reference Example 1.1, a total pore volume of 0.69 ml/g determined as described in Reference Example 1.9.

As determined according to Reference Example 1.12 and displayed in FIG. 10, 3% of the catalyst moldings have an aspect ratio in the range of from 1.0 to 1.5, 13% have an aspect ratio in the range of greater than 1.5 to 2.0, 20% have an aspect ratio in the range of greater than 2.0 to 2.5, 18% have an aspect ratio in the range of greater than 2.5 to 3.0, 16% have an aspect ratio in the range of greater than 3.0 to 3.5, 14% have an aspect ratio in the range of greater than 3.5 to 4.0, 5% have an aspect ratio in the range of greater than 4.0 to 4.5, 4% have an aspect ratio in the range of greater than 4.5 to 5.0, 5% have an aspect ratio in the range of greater than 5.0 to 5.5, and 4% have an aspect ratio greater than 5.5.

### Reference Example 6: Activation Factor of Reference Examples, Examples and Comparative Examples

For determining the activation factor of the prepared catalysts, the concentration of bridging μ² η²-peroxo species per Ti in the H₂O₂-activated catalyst (“mmol μ-peroxo species/mol Ti”) was determined for the examples and comparative examples according to the method described in Reference Example 1.2, wherein the concentration was determined 15 min and 2 h after having activated the respective samples with hydrogen peroxide. Based thereon, the activation factor was calculated according to Reference Example 1.10.

TABLE 1
Overview of relative values for H₂¹⁷O₂, bridging μ² η²-peroxo (“μ-peroxo”),
and H₂¹⁷O after 15 min and after 2 hours as obtained from the measurements and
application of the quantification protocol described in Reference Example 1.2.

| | Ti | | rel. μ- | | rel. | rel. μ- | rel. |
|---|---|---|---|---|---|---|---|
| | content | rel. H₂¹⁷O₂ | peroxo | rel. H₂¹⁷O | H₂¹⁷O₂ | peroxo | H₂¹⁷O |
| # | [wt.-%] | (15 min) | (15 min) | (15 min) | (2 h) | (2 h) | (2 h) |
| Ex. 2 | 0.9 | 0.25 | 0.44 | 0.31 | 0.08 | 0.51 | 0.41 |
| Ex. 3 | 1.0 | 0.13 | 0.59 | 0.28 | 0.06 | 0.60 | 0.35 |
| Ref. | 2.0 | 0.21 | 0.66 | 0.13 | 0.04 | 0.80 | 0.16 |
| Ex. 5 | | | | | | | |
| Comp. | 1.6 | 0.27 | 0.58 | 0.15 | 0.15 | 0.46 | 0.39 |
| Ex. 6 | | | | | | | |
| Ref. | 1.2 | 0.00 | 0.67 | 0.33 | 0.00 | 0.59 | 0.41 |
| Ex. 3 | | | | | | | |
| Ref. | 1.8 | 0.00 | 0.65 | 0.35 | 0.00 | 0.54 | 0.46 |
| Ex. 4 | | | | | | | |
| Ref. | 1.8 | 0.33 | 0.35 | 0.32 | 0.26 | 0.34 | 0.40 |
| Ex. 2.2 | | | | | | | |

TABLE 2
Overview of concentrations of H₂¹⁷O₂, bridging
μ² η²-peroxo (“μ-peroxo”), and H₂¹⁷O after
15 min as calculated according to Reference
Example 1.2 and based on the value of the
respective rel. concentration.

| | | conc. | conc. | conc. |
|---|---|---|---|---|
| | Ti | H₂¹⁷O₂ | μ-peroxo | H₂¹⁷O |
| | content | (15 min) | (15 min) | (15 min) |
| # | [wt.-%] | [μmol/mg] | [μmol/mg] | [μmol/mg] |
| Ex. 2 | 0.9 | 0.047 | 0.082 | 0.057 |
| Ex. 3 | 1.0 | 0.027 | 0.126 | 0.058 |
| Ref. Ex. 5 | 2.0 | 0.086 | 0.276 | 0.054 |
| Comp. | 1.6 | 0.091 | 0.197 | 0.050 |
| Ex. 6 | | | | |
| Ref. Ex. 3 | 1.2 | 0.000 | 0.166 | 0.083 |
| Ref. Ex. 4 | 1.8 | 0.000 | 0.247 | 0.131 |
| Ref. | 1.8 | 0.118 | 0.125 | 0.115 |
| Ex. 2.2 | | | | |

TABLE 3
Overview of concentrations of H₂¹⁷O₂, bridging
μ² η²-peroxo (“μ-peroxo”), and H₂¹⁷O after
15 min as calculated according to Reference
Example 1.2 and based on the value of the
respective rel. concentration.

| | | conc. | conc. | conc. |
|---|---|---|---|---|
| | Ti | H₂¹⁷O₂ | μ-peroxo | H₂¹⁷O |
| | content | (2 h) | (2 h) | (2 h) |
| # | [wt.-%] | [μmol/mg] | [μmol/mg] | [μmol/mg] |
| Ex. 2 | 0.9 | 0.015 | 0.094 | 0.077 |
| Ex. 3 | 1.0 | 0.012 | 0.126 | 0.073 |
| Ref. Ex. 5 | 2.0 | 0.015 | 0.333 | 0.068 |
| Comp. Ex. 6 | 1.6 | 0.048 | 0.153 | 0.129 |
| Ref. Ex. 3 | 1.2 | 0.000 | 0.147 | 0.103 |
| Ref. Ex. 4 | 1.8 | 0.000 | 0.203 | 0.174 |
| Ref. Ex. 2.2 | 1.8 | 0.093 | 0.122 | 0.143 |

TABLE 4
Activation factors after 15 min and results from
catalytic testing of the prepared catalysts.

| | | | ¹⁷O-NMR: |
|---|---|---|---|
| | | | mmol |
| | | | μ-peroxo/ |
| | activation | | mol |
| | factor | water | Ti |
| | (15 min) | adsorption | (15 min) |
| # | [mmol/mol] | [g/g] | [mmol/mol] |
| Ex. 2 | 20 | 0.046 | 444 |
| Ex. 3 | 34 | 0.057 | 601 |
| Ref. Ex. 5 | 76 | 0.115 | 660 |
| Comp. Ex. 6 | 61 | 0.104 | 588 |
| Ref. Ex. 3 | 29 | 0.044 | 663 |
| Ref. Ex. 4 | 46 | 0.07 | 655 |
| Ref. Ex. 2.2 | 21 | 0.063 | 333 |

TABLE 5
Activation factors after 2 h and results from
catalytic testing of the prepared catalysts.

| | | | ¹⁷O-NMR: |
|---|---|---|---|
| | | | mmol |
| | | | μ-peroxo/ |
| | activation | | mol |
| | factor | water | Ti |
| | (2 h) | adsorption | (15 min) |
| # | [mmol/mol] | [g/g] | [mmol/mol] |
| Ex. 2 | 24 | 0.046 | 512 |
| Ex. 3 | 34 | 0.057 | 601 |
| Ref. Ex. 5 | 91 | 0.115 | 795 |
| Comp. Ex. 6 | 47 | 0.104 | 456 |
| Ref. Ex. 3 | 26 | 0.044 | 584 |
| Ref. Ex. 4 | 38 | 0.07 | 655 |
| Ref. Ex. 2.2 | 20 | 0.063 | 323 |

### Example 12: Preliminary Test-PO Test

Moldings of the examples were preliminarily tested with respect to their general suitability as epoxidation catalysts according to the PO test as described in Reference Example 1.5. The respective resulting values of the propylene oxide activity are shown in Table 6 below.

TABLE 6
Results for catalytic testing according
to Reference Example 1.5.

| | | Time |
|---|---|---|
| | | for 10% |
| | propylene | pressure |
| Sample | oxide | loss in |
| according | activity | PO test |
| to # | [%] | [min] |
| Example 1.1 | 5.47 | 31 |
| Example 1.2 | 5.66 | 29 |
| Example 2 | 5.7 | 22 |
| Example 3 | 5.9 | 24 |
| Example 4 | 4.6 | 34 |
| Example 5 | 4.1 | 52 |
| Example 7 | 6.0 | 14 |
| Example 8 | 5.84 | 19 |
| Example 9 | 5.46 | 24 |
| Example 10 | 4.71 | 69 |
| Comparative | 4.5 | 35 |
| Example 6 | | |

Obviously, the moldings according to the present invention exhibit a very good propylene oxide activity according to the PO test and are promising candidates for catalysts in industrial continuous epoxidation reactions.

### Example 13: Preliminary Test-k-80 Test

Catalyst moldings of the examples were preliminarily tested with respect to their general suitability as epoxidation catalysts according to the k-80 test as described in Reference Example 1.6. The respective resulting values are shown in Table 7 below.

TABLE 7
Results for catalytic testing according
to Reference Example 1.6.

| Sample | k-80 |
|---|---|
| according | test |
| to # | [h⁻¹] |
| Example 1.1 | 0.15 |
| Example 1.2 | 0.06 |
| Example 2 | 0.16 |
| Example 3 | 0.089 |
| Example 5 | 0.053 |
| Example 7 | 0.05 |
| Example 8 | 0.14 |
| Example 9 | 0.11 |
| Example 10 | 0.19 |
| Comparative Example 6 | 0.20 |

Obviously, the moldings according to the present invention exhibit a very good activity according to the k-80 test and are promising candidates for catalysts in industrial continuous epoxidation reactions.

### Example 14: Catalytic Characteristics of the Moldings in a Continuous Epoxidation Reaction

In a continuous epoxidation reaction setup, a vertically arranged tubular reactor (length: 1.4 m, outer diameter 10 mm, internal diameter: 7 mm) equipped with a jacket for thermostatization was charged with 15 g of the moldings in the form of strands as described in the respective examples above. The remaining reactor volume was filled with inert material (steatite spheres, 2 mm in diameter) to a height of about 5 cm at the lower end of the reactor and the remainder at the top end of the reactor. Through the reactor, the starting materials were passed with the following flow rates: methanol (78 g/h); hydrogen peroxide (HP) (18.8 g/h; employed as aqueous hydrogen peroxide solution with a hydrogen peroxide content of 40 weight-%); propylene (10.8 g/h; polymer grade). Throughout the entire reaction process, a cooling medium was circulated through the cooling jacket. During the startup phase, the temperature of the cooling medium was set to 35 °C, and the initial conversion rate was typically above 90%. However, if the conversion rate dropped below 90%, the temperature of the reaction mixture was slowly adjusted by the cooling medium to maintain a constant conversion rate of 90%. The hydrogen peroxide conversion rate was determined by analyzing the reaction mixture leaving the reactor. The pressure within the reactor was held constant at 20 bar (abs), and the reaction mixture-apart from the fixed-bed catalyst-consisted of one single liquid phase.

The reactor effluent stream downstream the pressure control valve was collected, weighed and analyzed. Organic components were analyzed in two separate gas-chromatographs. The hydrogen peroxide content was determined colorimetrically using the titanyl sulfate method, preferably according to the determination method disclosed by George M. Eisenberg in Ind. Eng. Chem. Anal. Ed. 1943, vol. 15, no. 5, p. 327. The selectivity for propylene oxide given was determined relative to propylene and hydrogen peroxide and was calculated as 100 times the ratio of moles of propylene oxide in the effluent stream divided by the moles of propylene or hydrogen peroxide in the feed.

The characteristics of moldings of the present invention were compared with comparative moldings not in accordance with the present invention in a continuous epoxidation reaction as described hereinabove. The results are shown in tables 8 and 9 below as well as FIGS. 1-3.

TABLE 8
Results for experimental testing of prepared catalyst moldings in an epoxidation reaction of
propylene. The runtime was chosen to be in the range from 350 to 360 h. Selectivity (HP) means
the selectivity based on hydrogen peroxide consumption at the end of the indicated runtime.
Selectivity (C3) means the selectivity based on propylene consumption at the end of the indicated
runtime. Selectivity based on HP is typically lower due to oxygen formation.

| | | | | | | Selectivity | Selectivity | Deactivation |
|---|---|---|---|---|---|---|---|---|
| | Runtime | Conversion | Temp. | Selectivity | Selectivity | (1-MOP-2) | (2-MOP-1) | rate |
| # | [h] | [%] | [°C] | (HP) [%] | (C3) [%] | [%] | [%] | [K/h] |
| Ex. 7 | 358 | 93.0 | 35 | 98.1 | 98.7 | 0.4 | 0.4 | 0 |
| Ex. 1.2 | 358 | 97.2 | 46 | 96.7 | 97.5 | 0.8 | 0.9 | 0.031 |
| Ex. 3 | 359 | 92.6 | 36 | 98.2 | 98.8 | 0.5 | 0.3 | 0.003 |
| Ex. 9 | 359 | 91.1 | 35 | 97.8 | 98.6 | 0.5 | 0.4 | 0 |
| Ex. 8 | 358 | 91.7 | 36 | 97.6 | 98.3 | 0.6 | 0.5 | 0.003 |
| Ex. 10 | 358 | 91.3 | 42 | 95.3 | 96.8 | 0.9 | 1.0 | 0.020 |

The deactivation rate was calculated according to formula II:

deactivation rate=delta T/delta t (II).

wherein delta T=T₁−T₀, and wherein delta t=t₁−t₀, wherein T₁ is the temperature of the cooling medium at a point in time t₁ during the runtime, and T₀ refers to a temperature of the cooling medium at a point in time to, wherein to refers to the point in time when the feed stream has reached for the first time the full load of hydrogen peroxide, wherein T₀ was presently set to 35 °C. The point in time t₀ refers to the time indicated in table 8.

Thus, the deactivation rate is defined as the average temperature increase required per hour to keep the conversion of hydrogen peroxide at or above 90% for the runtime indicated in table 8.

The selectivity(S) towards a compound (X) in % was calculated according to following formula III, wherein compound X particularly relates to 1-MOP-2 or 2-MOP-1, respectively, or to the sum of 1-MOP-2 and 2-MOP-1:

S(X)[%]=[mmol compound X(effluent) per hour/(mmol HP(feed) per hour-mmol HP(effluent) per hour]*100% (III).

TABLE 9
Selectivity towards methoxypropanol-
compounds after 24 h.

| | | Selectivity | Selectivity |
|---|---|---|---|
| | Runtime | (1-MOP-2) | (2-MOP-1) |
| # | [h] | [%] | [%] |
| Ex. 7 | 22 | 1.12 | 2.63 |
| Ex. 1.2 | 24 | 0.76 | 0.95 |
| Ex. 3 | 23 | 1.15 | 2.04 |
| Ex. 9 | 23 | 1.57 | 4.54 |
| Ex. 8 | 22 | 1.57 | 4.56 |
| Ex. 10 | 23 | 0.95 | 1.01 |

1-MOP-2: 1-methoxy-2-propanol
2-MOP-1: 2-methoxy-1-propanol

It was particularly found that use of a catalyst molding according to the present invention in the epoxidation reaction achieved a similar conversion than a state-of-the-art catalyst molding while showing a lower selectivity towards by-products 1-MOP-2 and 2-MOP-1, and a lower deactivation rate after a full runtime of about 360 h.

Obviously, the inventive moldings show highly advantageous improved lifetime characteristics in a continuous epoxidation reaction, wherein this continuous mode is the standard mode for industrial-scale epoxidation processes.

### BRIEF DESCRIPTION OF FIGURES

FIG. 1: shows the catalytic performance of the moldings of the present invention according to Examples 1.2, 3, and 7-10 (filled circles (●) refers to Ex. 7, crosses (x) refer to Ex. 1.2, open squares (□) refer to Ex. 3, open triangles (Δ) refer to Ex. 9, capitalized Latin letter y (Y) refer to Ex. 8, and Capitalized Latin letter z (Z) refer to Ex. 10). In the upper part, the hydrogen peroxide (HP) conversion is shown on the ordinate in % relative to the runtime on the abscissa in h. In the lower part, the temperature is shown on the ordinate in °C relative to the runtime on the abscissa in h.

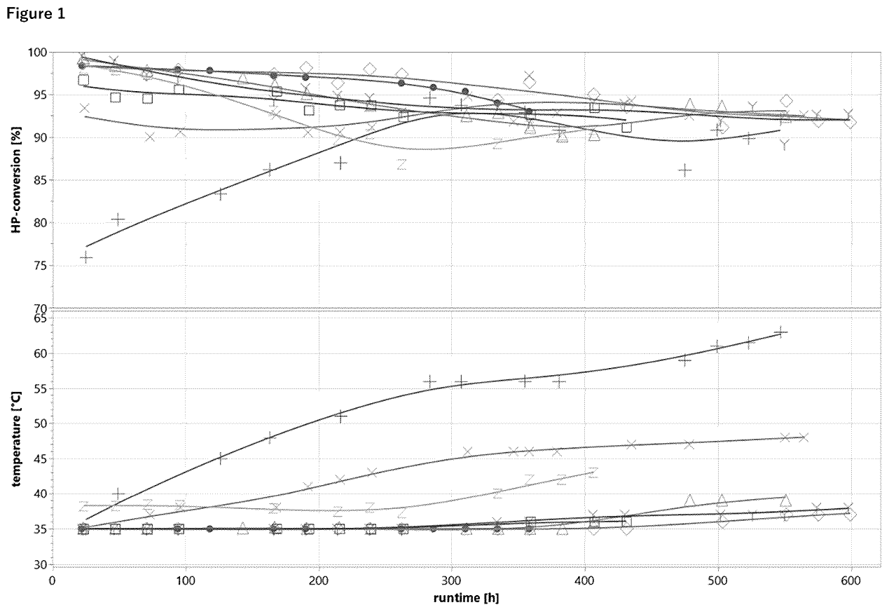

FIG. 2: shows the catalytic performance of the moldings of the present invention according to Examples 1.2, 3, and 7-10 (filled circles (●) refers to Ex. 7, crosses (x) refer to Ex. 1.2, open squares (□) refer to Ex. 3, open triangles (Δ) refer to Ex. 9, capitalized Latin letter y (Y) refer to Ex. 8, and Capitalized Latin letter z (Z) refer to Ex. 10). In the upper part, the propylene oxide selectivity relative to hydrogen peroxide is shown on the ordinate in % relative to the runtime on the abscissa in h. In the middle part, the 1-MOP-2 selectivity relative to hydrogen peroxide is shown on the ordinate in % relative to the runtime on the abscissa in h. In the lower part, the 2-MOP-1 selectivity relative to hydrogen peroxide is shown on the ordinate in % relative to the runtime on the abscissa in h.

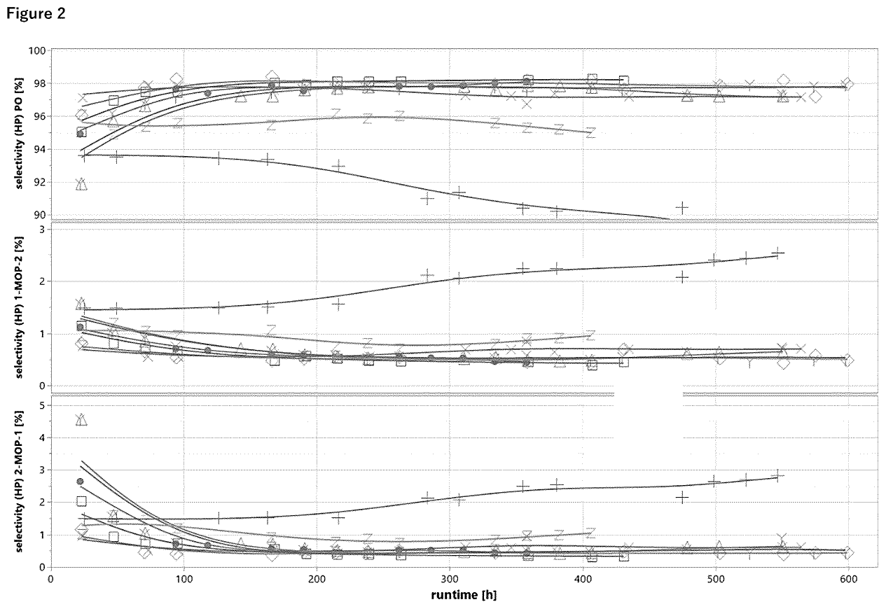

FIG. 3: shows the catalytic performance of the moldings of the present invention according to Examples 1.2, 3, and 7-10 (filled circles (●) refers to Ex. 7, crosses (x) refer to Ex. 1.2, open squares (□) refer to Ex. 3, open triangles (Δ) refer to Ex. 9, capitalized Latin letter y (Y) refer to Ex. 8, and Capitalized Latin letter z (Z) refer to Ex. 10). In the upper part, the propylene oxide selectivity relative to hydrogen peroxide is shown on the ordinate in % relative to the runtime on the abscissa in h. In the lower part, the propylene oxide selectivity relative to propylene is shown on the ordinate in % relative to the runtime on the abscissa in h.

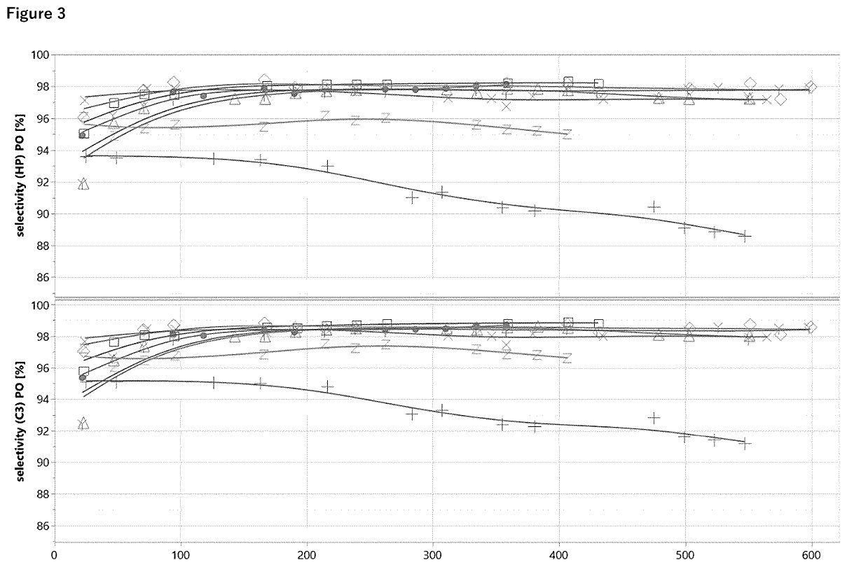

FIG. 4: shows the distribution of the aspect ratios of the catalyst moldings of Example 1.1, wherein the range of aspect ratios for which each bar of the histogram stands is indicated in the abscissa, and the number of moldings among the 90 moldings which were measured falling within the given range of aspect ratios is indicated at the top of each bar.

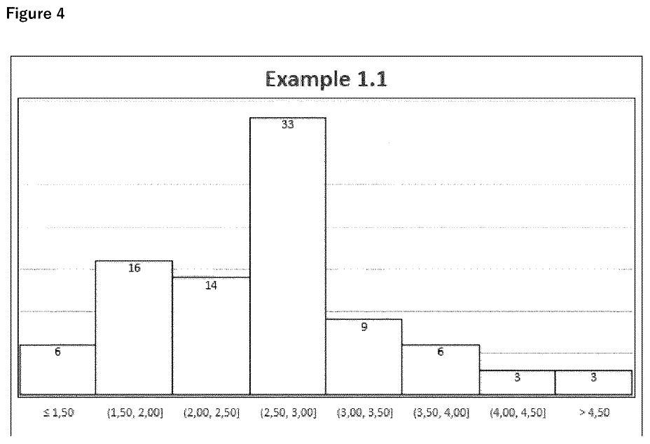

FIG. 5: shows the distribution of the aspect ratios of the catalyst moldings of Example 1.2, wherein the range of aspect ratios for which each bar of the histogram stands is indicated in the abscissa, and the number of moldings among the 90 moldings which were measured falling within the given range of aspect ratios is indicated at the top of each bar.

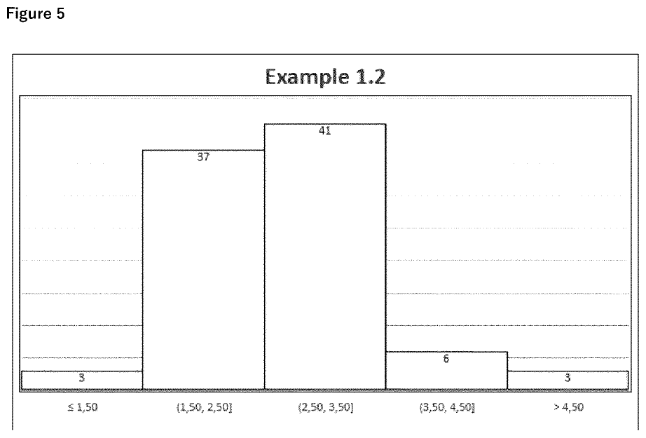

FIG. 6: shows the distribution of the aspect ratios of the catalyst moldings of Example 2, wherein the range of aspect ratios for which each bar of the histogram stands is indicated in the abscissa, and the number of moldings among the 155 moldings which were measured falling within the given range of aspect ratios is indicated at the top of each bar.

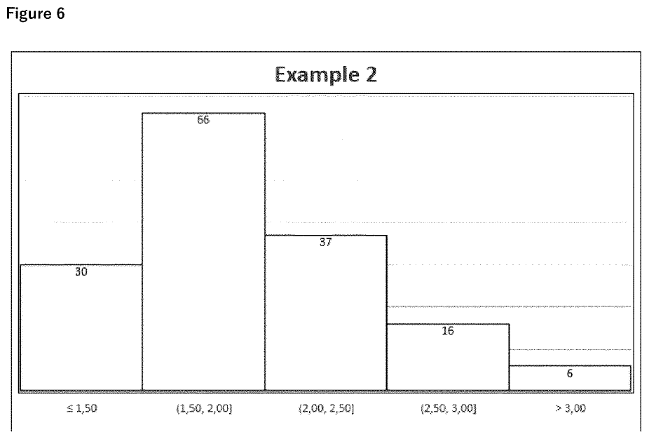

FIG. 7: shows the distribution of the aspect ratios of the catalyst moldings of Example 3, wherein the range of aspect ratios for which each bar of the histogram stands is indicated in the abscissa, and the number of moldings among the 77 moldings which were measured falling within the given range of aspect ratios is indicated at the top of each bar.

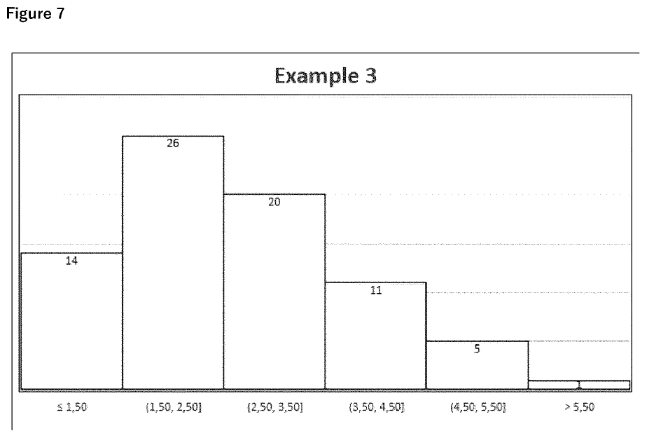

FIG. 8: shows the distribution of the aspect ratios of the catalyst moldings of Example 5, wherein the range of aspect ratios for which each bar of the histogram stands is indicated in the abscissa, and the number of moldings among the 212 moldings which were measured falling within the given range of aspect ratios is indicated at the top of each bar.

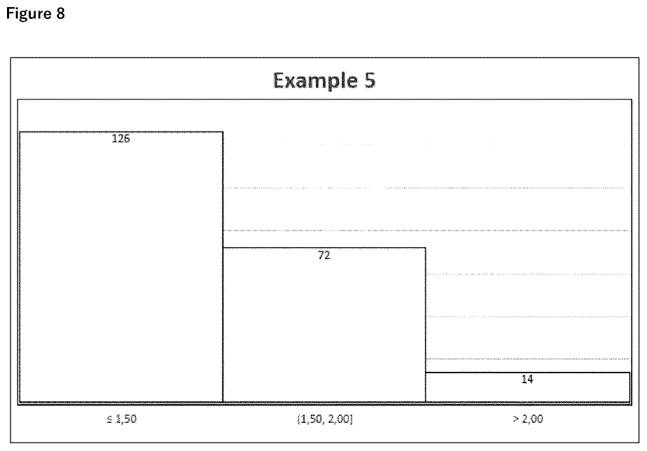

FIG. 9: shows the distribution of the aspect ratios of the catalyst moldings of Comparative Example 6, wherein the range of aspect ratios for which each bar of the histogram stands is indicated in the abscissa, and the number of moldings among the 229 moldings which were measured falling within the given range of aspect ratios is indicated at the top of each bar.

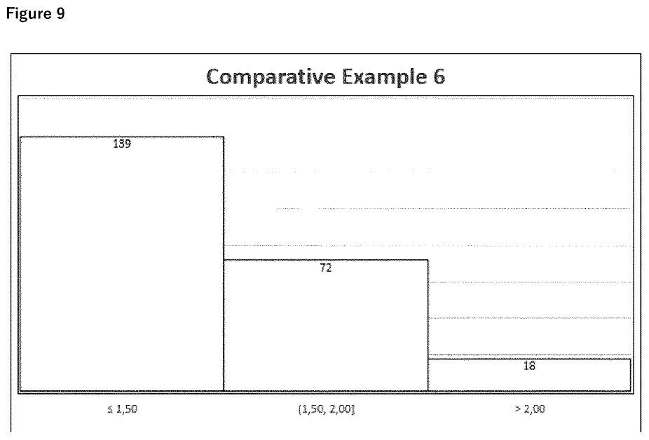

FIG. 10: shows the distribution of the aspect ratios of the catalyst moldings of Example 10, wherein the range of aspect ratios for which each bar of the histogram stands is indicated in the abscissa, and the number of moldings among the 111 moldings which were measured falling within the given range of aspect ratios is indicated at the top of each bar.

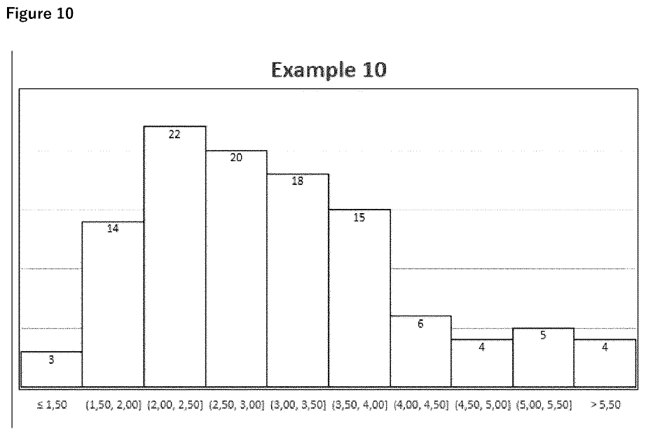

### CITED LITERATURE

WO 2020/074586 A1
US 2015/0118149 A1
CN 115974094 A
CN 115920958 A
WO 2015/029055 A1
R. Wang et al. “Fundamental Understanding and Catalytic Applications of Hollow MFI-type Zeolites” in Catalysis Today 2022
Langerame, F., Salvi, A. M., Silletti, M. and Moretti, G. (2008) “XPS characterization of a synthetic Ti-containing MFI zeolite framework: the titanosilicalites, TS-1” in Surf. Interface Anal., 40:695-699; https://doi.org/10.1002/sia.2739
Fairley N, (2011); CASA-XPS, 2.3.25ed., Casa Software Ltd.
C. A. Schneider, W. S. Rasband and K. W. Eliceiri: NIH Image to ImageJ: 25 years of image analysis in Nature Methods, volume 9, 2012, p. 671-675; doi: 10.1038/nmeth.208
George M. Eisenberg in Ind. Eng. Chem. Anal. Ed. 1943, vol. 15, no. 5, p. 327
I. C. Madsen and N. V. Y. Scarlett, Chapter 11.3.2.1, in Powder Diffraction-Theory and Practice edited by R. E. Dinnebier and S. J. L. Billinge
Lamberti C., Bordiga S., Zecchina A., Carati A., Fitch A. N., Artioli G., Petrini G., Salvalaggio M., Marra G. L. “Structural Characterization of Ti-Silicalite-1: A Synchrotron Radiation X-Ray Powder Diffraction Study” in J. Catal. 1999, 183, 222-231

1.-15. (canceled)

16. A reactor comprising a plurality of catalyst moldings, each of the catalyst moldings independently from one another being a catalyst molding comprising a zeolitic material having framework type MFI,
wherein the catalyst molding has an average crush strength in the range of from 3 to 30 N,
wherein the crushing strength is determined according to Reference Example 1.7,
wherein the catalyst molding has an aspect ratio D1:D2,
wherein D1 stands for the largest distance separating a pair of parallel planes P1 and P2 when the catalyst molding is located in-between said parallel planes without transgressing them, and the catalyst molding is in contact with each of the respective planes in at least one point, and
wherein D2 stands for the shortest distance separating a pair of parallel planes P3 and P4 when the catalyst molding is located in-between said parallel planes without transgressing them, and the catalyst molding is in contact with each of the respective planes in at least one point,
wherein the aspect ratio D1:D2 is equal to or greater than 1:1;
wherein from 42 to 100% of the plurality of catalyst moldings has an aspect ratio in the range of from greater than 1.5 to smaller than 6.1; and
wherein from 10 to 46% the plurality of catalyst moldings has an aspect ratio in the range of from greater than 1.5 to 2.0.

17. The reactor of claim 16, wherein D1 is in the range of from 0.1 to 10 mm, or wherein D2 is in the range of from 0.05 to 5 mm.

18. The reactor of claim 16, wherein the catalyst molding has a total pore volume in the range of from 0.60 to 1.2 ml/g.

19. The reactor of claim 16, wherein the catalyst molding exhibits a water adsorption in the range of from 1.0 to 15.0 weight-%.

20. The reactor of claim 16, wherein the catalyst molding has a Ti content in the range of from 0.4 to 1.85 weight-%, calculated as elemental Ti and based on the sum of the weights of the zeolitic material and optionally the one or more oxidic binders.

21. The reactor of claim 16, wherein the catalyst molding displays a water adsorption (W), determined according to Reference Example 1.1, a concentration (C) of bridging μ²η²-peroxo species per Ti in the H₂O₂-activated catalyst molding, as determined according to Reference Example 1.2, wherein the concentration (C) is the concentration which is determined at a time point T after having brought the catalyst molding into contact with H₂¹⁷O₂, wherein T is in the range of from 1 to 720 min, and an activation factor (A) according to formula I, wherein the activation factor is in the range of from 10 to 75 mmol/mol;
wherein in accordance with formula I, the activation factor is the multiplication product of the water adsorption and the concentration of bridging μ²η²-peroxo species per Ti in the H₂O₂-activated catalyst molding:
A=W×C (I)

22. The reactor of claim 16, wherein the catalyst molding has a BET specific surface area in the range of from 200 to 450 m²/g.

23. The reactor of claim 16, wherein the catalyst molding shows a selectivity towards the sum of 1-methoxy-2-propanol and 2-methoxy-1-propanol in the range of from 0 to 15%, wherein the deactivation rate is determined according to Example 14.

24. The reactor of claim 16, wherein the catalyst molding shows a deactivation rate in the range of from 0 to 0.055 K/h, wherein the deactivation rate is determined as described in Example 14.

25. The reactor of claim 16, wherein the catalyst molding has a bulk density of equal to or greater than 320 g/l, wherein the bulk density is determined according to Reference Example 1.11.

26. A process for the activation of hydrogen peroxide comprising:
(1) providing a reactor comprising a plurality of catalyst moldings according to claim 16;
(2) contacting the catalyst molding provided in (1) or the plurality of catalyst moldings comprised in the reactor provided in (1) with hydrogen peroxide.

27. The reactor according to claim 16, wherein the reactor is used for carrying out a reaction involving one or more of C—O bond formation, C—C bond formation and C—C bond conversion.

28. A process for preparing an olefin oxide comprising
(A) providing olefin, hydrogen peroxide, water and organic solvent and optionally an additive into an epoxidation zone comprising the reactor according to claim 16, obtaining a reaction mixture comprising olefin, hydrogen peroxide, water and organic solvent;
(B) subjecting the reaction mixture obtained from (A) to epoxidation reaction conditions in the epoxidation zone, thereby obtaining a mixture comprising olefin oxide, water and organic solvent;
(C) removing an effluent stream from the epoxidation zone, comprising olefin oxide, water and organic solvent.
